(19) United States
(12) Patent Application Publication
(10) Pub. No.: US 2026/0240976 A1
(43) Pub. Date: Aug. 20, 2026

(54) THERAPEUTIC HPV VACCINES BASED ON VALIDATED TARGET EPITOPES

(71) Applicant: Deutsches Krebsforschungszentrum Stiftung des öffentlichen Rechts, Heidelberg (DE)

(72) Inventors: Angelika Beate RIEMER, Heidelberg (DE); Maria BONSACK, Heidelberg (DE); Jonas FÖRSTER, Heidelberg (DE); Jonas BECKER, Heidelberg (DE); Kathrin WELLACH, Heidelberg (DE)

(73) Assignee: Deutsches Krebsforschungszentrum Stiftung des öffentlichen Rechts, Heidelberg (DE)

(21) Appl. No.: 19/160,842

(22) PCT Filed: Mar. 1, 2024
(86) PCT No.: PCT/EP2024/055429

§ 371 Date: Aug. 28, 2025

(30) Foreign Application Priority Data
Mar. 2, 2023 (EP) 23159739.4

(51) Int. Cl. A61K 39/12; A61K 39/00; A61P 37/04

(52) U.S. Cl. CPC A61K 39/12; A61P 37/04; A61K 2039/70

(57) ABSTRACT

The present invention relates to a vaccine for use in therapeutic vaccination of a human subject against a human papillomavirus 16 (HPV16)-related virus, wherein said vaccine provides at least five discrete immunization peptides consisting of amino acid sequences selected from at least five of the following groups: (i) SEQ ID NOs: 1 to 10; (ii) SEQ ID NOs: 11 to 52; (iii) SEQ ID NOs:53 to 79; (iv) SEQ ID NOs: 71 and 80 to 96; (v) SEQ ID NOs:25, 81, and 97-106; and (vi) SEQ ID NOs: 9, 12, 23, 63, 81, 85, 101, and 107 to 131; wherein said vaccine is a human leukocyte antigen (HLA) universal vaccine for use in any human subject expressing at least one HLA of a HLA supertype selected from the list consisting of HLA-A01, HLA-A02, HLA-A03/A11, HLA-A24, HLA-B07, and HLA-B15, and wherein each of said at least five immunization peptides, when presented as an H LA-complex, activates human anti-immunization peptide T cells. The present invention also relates to kits, devices, and methods related thereto.

The present invention relates to a vaccine for use in therapeutic vaccination of a human subject against a human papillomavirus 16 (HPV16)-related virus, wherein said vaccine provides at least five discrete immunization peptides consisting of amino acid sequences selected from at least five of the following groups: (i) SEQ ID NOs: 1 to 10; (ii) SEQ ID NOs:11 to 52; (iii) SEQ ID NOs:53 to 79; (iv) SEQ ID NOs: 71 and 80 to 96; (v) SEQ ID NOs:25, 81, and 97-106; and (vi) SEQ ID NOs: 9, 12, 23, 63, 81, 85, 101, and 107 to 131; wherein said vaccine is a human leukocyte antigen (HLA) universal vaccine for use in any human subject expressing at least one HLA of a HLA supertype selected from the list consisting of HLA-A01, HLA-A02, HLA-A03/A11, HLA-A24, HLA-B07, and HLA-B15, and wherein each of said at least five immunization peptides, when presented as an HLA-complex, activates human anti-immunization peptide T cells. The present invention also relates to kits, devices, and methods related thereto.

At least 20% of human malignancies are caused by consequences of persistent infections. Cancers caused by infectious agents are attractive targets for cancer vaccination approaches, as they provide the opportunity to target antigens that are immunological non-self. Vaccination can be prophylactic, inducing an immune response that prevents infection in the first place, or therapeutic, stimulating the immune system into eradicating established disease. Prophylactic immunization against certain high-risk human papillomavirus (HPV) types or against hepatitis B has become the paradigm for cancer immunoprevention.

As for therapeutic vaccination, HPV-associated neoplasia could again emerge as the model case: induction and maintenance of the malignant phenotype depends on two viral oncoproteins, E6 and E7. It is known from studies on spontaneously regressing HPV-induced lesions, and from HPV prevalence in immunosuppressed and HIV-infected patients, that cell-mediated immune responses are crucial in clearing established HPV infection. Various forms of vaccines, including peptide vaccines, targeting T cells to E6 and E7 have already been explored.

Candidate peptides proposed for vaccination against HPV are abundant in the literature, e.g. from Bonsack et al. (2019), Cancer Immunol Res; 7(5):719; Blatnik (2018), Proteomics 18:1700390, DOI: 10.1002/pmic.201700390; Krishna et al. (2018) Cancer Res. 2018; 78(21):6159; Tsang et al. (2017), Vaccine 35(19):2605; Kast et al., J Immunol 1994; 152(8):3904; Ressing et al., J Immunol 1995; 154(11):5934; Bourgault Villada et al., Clin Exp Immunol 2010; 159(1):45; Mizuuchi et al., Exp Mol Pathol 2012; 92(1):185); Hara et al., Int J Oncol 2005; 27(5):1371; Jang et al., Cancer 2012; 118(8):2173, and Riemer et al., J Biol Chem 2010; 285(38):29608. Regarding peptide vaccines, it had, however, to be realized over the years that the processes leading to an effective T cell response are complex and require more than potential binding of a peptide to an MHC. In the case of MHC class I presentation, a precursor peptide must be cleaved by the proteasome of a presenting cell to provide the peptide in the first place; moreover, T cell activation may require cross-presentation of the peptide on MHC class I by APCs, which may cleave precursor peptides differently from non-APC cells. Moreover, there must be a T cell with the propensity to recognize the presented peptide, which crucially depends on the reservoir of T cell receptors available (cf. e.g. Becker & Riemer (2022), Frontiers Immunol, doi: 10.3389/fimmu.2022.883989; Habib et al. (2022), Cells 6; 11(3):421. doi: 10.3390/cells11030421.). Also, in cancer cells, immune evasion mechanisms may prevent proper presentation of HPV-derived peptides (Steinbach & Riemer (2018), Int J Cancer 142:224). In conclusion, experimental binding of a candidate peptide to MHC class I in itself, unfortunately, does not make this candidate peptide already a plausible candidate for a vaccine.

In view of the above, it is clear that verifying candidate HPV peptides as indeed suitable for vaccination is by far a non-routine task: in presentation assays, the amounts of peptide obtainable are minute, and for many peptides, fragmentation patterns in mass spectrometry are complex. It is, thus, that in a study of 2010 (Riemer et al. (2010), loc.cit.), of 21 peptides predicted to be presented via HLA-A*0201, only one could be confirmed to be presented. After eight years of method development, 17 peptides could be confirmed from a predicted group of 121 potential HLA-A2 binding peptides (Blatnik et al. (2018), loc. cit.). Notably, cysteine-containing peptides were excluded altogether, as is usual in the art, since they are prone to intra- and intermolecular reactions, which complicate analysis and further reduce the amounts of defined and detectable ions available for MS analysis. If a HPV peptide cannot be identified as suitable by the in vitro methods described above, the only remaining alternative is in vivo testing, which requires extended animal and/or human testing by vaccination.

Also, for successful therapeutic vaccination against HPV, it is not only necessary that the vaccinated peptide is immunogenic, e.g. by activating T cells when presented via MHC molecules, but the peptide also has to be presented by target cells, i.e. HPV-infected cells. As the skilled person is aware of, showing that a peptide externally added to MHC-presenting cells can activate T cells cannot show that the peptide is presented by target cells. As noted in Riemer et al. ((2010), loc. cit.), it is essential for vaccination to define HPV-16 E6 and E7 T cell epitopes that are naturally processed and presented on the surface of virally altered cells. Only those HPV peptide/MHC class I complexes are capable of being recognized by cytolytic T lymphocytes to target destruction of transformed cells. Thus, success of a therapeutic HPV vaccine is dependent on accurate identification of HPV epitopes displayed on HPV-infected cells. Apart from direct proof of MHC presentation by MS analysis, investigating memory responses in healthy donors can serve as a surrogate test for presentation. If memory immune responses are present, the epitope in question must have been presented during a previous encounter with the virus. However, even proven natural immunogenicity is not sufficient to validate a peptide in the context of its restricting HLA complex as an actionable target. This is because APCs use a different proteasome, the immunoproteasome, compared to normal body cells, and furthermore, antigen processing of HPV16-infected/transformed cells is heavily modulated by the virus. Thus, a memory T cell response only proves that the peptide was presented by antigen-presenting cells (APCs), not that this epitope will still be presented on HPV-dependent tumor cells. Thus, not every possible HPV epitope is presented on HPV-positive target cells.

Thus, to be an actionable target, a HPV16-derived peptide must bind to an HLA molecule, be immunogenic, and must also be presented on the target cell, in particular an HPV16 infected or transformed cell.

There is, thus, a need in the art for improved means and methods for immunoreactive agents against cells transformed by HPV, such as HPV-positive cancer cells, and reagents related thereto, without the drawbacks as referred to above.

The technical problem underlying the present invention can be seen as the provision of means and methods for complying with the aforementioned needs. The technical problem is solved by the embodiments characterized in the claims and herein below.

In accordance, the present invention relates to a vaccine providing at least one discrete immunization peptide consisting of an amino acid sequence selected from at least one of the following groups:
(i) SEQ ID NOs: 1 to 10;
(ii) SEQ ID NOs:11 to 52;
(iii) SEQ ID NOs:53 to 79;
(iv) SEQ ID NOs: 71 and 80 to 96;
(v) SEQ ID NOs:25, 81, and 97-106; and
(vi) SEQ ID NOs: 9, 12, 23, 63, 81, 85, 101, and 107 to 131.

In general, terms used herein are to be given their ordinary and customary meaning to a person of ordinary skill in the art and, unless indicated otherwise, are not to be limited to a special or customized meaning. As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements. Also, as is understood by the skilled person, the expressions “comprising a” and “comprising an” preferably refer to “comprising one or more”, i.e. are equivalent to “comprising at least one”. In accordance, expressions relating to one item of a plurality, unless otherwise indicated, preferably relate to at least one such item, more preferably a plurality thereof; thus, e.g. identifying “a cell” relates to identifying at least one cell, preferably to identifying a multitude of cells.

Further, as used in the following, the terms “preferably”, “more preferably”, “most preferably”, “particularly”, “more particularly”, “specifically”, “more specifically” or similar terms are used in conjunction with optional features, without restricting further possibilities. Thus, features introduced by these terms are optional features and are not intended to restrict the scope of the claims in any way. The invention may, as the skilled person will recognize, be performed by using alternative features. Similarly, features introduced by “in an embodiment” or similar expressions are intended to be optional features, without any restriction regarding further embodiments of the invention, without any restrictions regarding the scope of the invention and without any restriction regarding the possibility of combining the features introduced in such way with other optional or non-optional features of the invention.

The methods specified herein below, preferably, are in vitro methods. The method steps may, in principle, be performed in any arbitrary sequence deemed suitable by the skilled person, but preferably are performed in the indicated sequence; also, one or more, preferably all, of said steps may be assisted or performed by automated equipment. Moreover, the methods may comprise steps in addition to those explicitly mentioned above.

As used herein, if not otherwise indicated, the term “about” relates to the indicated value with the commonly accepted technical precision in the relevant field, preferably relates to the indicated value ±20%, more preferably ±10%, most preferably ±5%. Further, the term “essentially” indicates that deviations having influence on the indicated result or use are absent, i.e. potential deviations do not cause the indicated result to deviate by more than ±20%, more preferably ±10%, most preferably ±5%. Thus, “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Preferably, a composition consisting essentially of a set of components will comprise less than 5% by weight, more preferably less than 3% by weight, even more preferably less than 1% by weight, most preferably less than 0.1% by weight of non-specified component(s).

The degree of identity (e.g. expressed as “% identity”) between two biological sequences, preferably DNA, RNA, or amino acid sequences, can be determined by algorithms well known in the art. Preferably, the degree of identity is determined by comparing two optimally aligned sequences over a comparison window, where the fragment of sequence in the comparison window may comprise additions or deletions (e.g., gaps or overhangs) as compared to the sequence it is compared to for optimal alignment. The percentage is calculated by determining, preferably over the whole length of the polynucleotide or polypeptide, the number of positions at which the identical residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman (1981), by the homology alignment algorithm of Needleman and Wunsch (1970), by the search for similarity method of Pearson and Lipman (1988), by computerized implementations of these algorithms (GAP, BESTFIT, BLAST, PASTA, and TFASTA in the Wisconsin Genetics Software Package, Genetics Computer Group (GCG), 575 Science Dr., Madison, WI), or by visual inspection. Given that two sequences have been identified for comparison, GAP and BESTFIT are preferably employed to determine their optimal alignment and, thus, the degree of identity. Preferably, the default values of 5.00 for gap weight and 0.30 for gap weight length are used. In the context of biological sequences referred to herein, the term “essentially identical” indicates a % identity value of at least 80%, preferably at least 90%, more preferably at least 98%, most preferably at least 99%. As will be understood, the term essentially identical includes 100% identity. The aforesaid applies to the term “essentially complementary” mutatis mutandis.

The term “fragment” of a biological macromolecule, preferably of a polynucleotide or polypeptide, is used herein in a wide sense relating to any sub-part, preferably subdomain, of the respective biological macromolecule comprising the indicated sequence, structure and/or function. Thus, the term includes sub-parts generated by actual fragmentation of a biological macromolecule, but also sub-parts derived from the respective biological macromolecule in an abstract manner, e.g. in silico. Thus, as used herein, an Fc or Fab fragment, but also e.g. a single-chain antibody, a bispecific antibody, and a nanobody may be referred to as fragments of an immunoglobulin.

Unless specifically indicated otherwise herein, the compounds specified, in particular the polynucleotides and (poly)peptides, may be comprised in larger structures, e.g. may be covalently or non-covalently linked to further sequences, carrier molecules, retardants, and other excipients. In particular, peptides and (poly)peptides as specified may be comprised in fusion polypeptides comprising further peptides, which may serve e.g. as a tag for purification and/or detection, as a linker, or to extend the in vivo half-life of a compound. The term “detectable tag” refers to a stretch of amino acids which are added to or introduced into the fusion polypeptide; preferably, the tag is added C- or N-terminally to the fusion polypeptide. Said stretch of amino acids preferably allows for detection of the polypeptide by an antibody which specifically recognizes the tag; or it preferably allows for forming a functional conformation, such as a chelator; or it preferably allows for visualization, e.g. in the case of fluorescent tags. Preferred detectable tags are the Myc-tag, FLAG-tag, 6-His-tag, HA-tag, GST-tag or a fluorescent protein tag, e.g. a GFP-tag. These tags are all well known in the art. Other further peptides preferably comprised in a fusion polypeptide comprise further amino acids or other modifications which may serve as mediators of secretion, as mediators of blood-brain-barrier passage, as cell-penetrating peptides, and/or as immune stimulants. Further polypeptides or peptides to which the polypeptides may be fused are signal and/or transport sequences, e.g. an IL-2 signal sequence, and linker sequences.

The term “polypeptide”, as used herein, refers to a molecule consisting of several, typically at least 20 amino acids that are covalently linked to each other by peptide bonds. Molecules consisting of less than 20 amino acids covalently linked by peptide bonds are usually considered to be “peptides”. The peptides referred to herein are disclosed as SEQ ID NOs:1 to 131, as well as in Table 1 herein below. As the skilled person understands from the disclosure herein, the immunization peptides described herein are preferably used with the HLA supertypes indicated herein in Tables 1(i) to (vi).

The term “polynucleotide” is known to the skilled person. As used herein, the term includes nucleic acid molecules comprising or consisting of a nucleic acid sequence or nucleic acid sequences as specified herein and/or encoding at least one immunization peptide. The polynucleotide of the present invention shall be provided, preferably, either as an isolated polynucleotide (i.e. isolated from its natural context) or in genetically modified form. The polynucleotide, preferably, is DNA, including cDNA, or is RNA. The term encompasses single as well as double stranded polynucleotides, as well as circularly closed and linear polynucleotides. Preferably, the polynucleotide is a chimeric molecule, i.e., preferably, comprises at least one nucleic acid sequence, preferably of at least 20 bp, more preferably at least 100 bp, heterologous to the residual nucleic acid sequence(s) or being an artificial nucleic acid sequence. Moreover, preferably, comprised are also chemically modified polynucleotides including naturally occurring modified polynucleotides such as glycosylated or methylated polynucleotides or artificial modified ones such as biotinylated polynucleotides.

The polynucleotide may be comprised in an expression construct. The term “expression construct”, as used herein, refers to a heterologous polynucleotide comprising the aforementioned polynucleotide as well as nucleic acid sequences required for expression of the polynucleotide. Typically, such additional nucleic acid sequences, which preferably are heterologous to the polynucleotide encoding the at least one immunization peptide, may be promoter sequences, regulatory sequences and/or transcription termination sequences, such as terminators. Preferably, the expression construct is a eukaryotic expression construct, i.e. an expression construct comprising all elements required for expression, preferably inducible expression, in a eukaryotic host cell. Suitable expression control sequences are well known in the art and include in particular the CMV promoter or other constitutive promoters. However, inducible and/or cell-type specific promoters may be used as well.

The polynucleotide and/or the expression construct may be comprised in a vector. The term “vector”, as used herein, relates to any composition of matter adapted for transferring and/or stably maintaining the polynucleotide and/or the expression construct as specified herein above in a host cell. Thus, the term vector preferably encompasses phage, plasmid, and viral vectors as well artificial chromosomes, such as bacterial or yeast artificial chromosomes. Preferably, the vector is a plasmid or a virus-derived vector, preferably a replication-incompetent viral vector. Moreover, the term also relates to targeting constructs which allow for random or site-directed integration of the targeting construct into genomic DNA of a host cell. Such target constructs, preferably, comprise DNA of sufficient length for either homologous or heterologous recombination. The vector encompassing the polynucleotide and/or the expression construct as specified herein above, preferably, further comprises at least one selectable marker for propagation and/or selection of a host cell. The vector may be incorporated into a host cell by various techniques well known in the art. For example, a plasmid vector can be introduced in a precipitate such as a calcium phosphate precipitate or rubidium chloride precipitate, or in a complex with a charged lipid or in carbon-based clusters, such as fullerenes. Alternatively, a plasmid vector may be introduced by heat shock or electroporation techniques. Should the vector be a virus, the polynucleotide may be packaged in vitro using an appropriate packaging cell line prior to application to host cells. Preferably, the vector is a vertebrate vector, more preferably a mammalian vector, or a shuttle vector. Preferably, the vector is an expression vector and/or a gene transfer or targeting vector. Methods which are well known to those skilled in the art can be used to construct recombinant polynucleotides and vectors; see, for example, the techniques described in Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1994). Preferably, the vector is an AAV vector, or a lentiviral vector.

The terms “human papillomavirus 16-related virus” and “HPV16-related virus” relate to any virus, preferably infecting humans, encoding at least one amino acid sequence of SEQ ID NOs: 1 to 131. Preferably, the HPV16-related virus is a papillomavirus (PV), more preferably a human papillomavirus (HPV), still more preferably is selected from the list consisting of HPV16, HPV31, HPV33, HPV35, HPV52, HPV58, and HPV67, most preferably is HPV16.

The term “immunization peptide”, as used herein, relates to a discrete peptide consisting of an amino acid sequence selected from SEQ ID NOs:1 to 131, preferably from SEQ ID NOs: 1 to 2; SEQ ID NOs:11 to 23; SEQ ID NOs:53 to 57; SEQ ID NOs: 80 to 81; SEQ ID NOs: 81, 97, and 98 to; and SEQ ID NOs: 81, and 107-114. Preferably, the immunization peptide has been verified to activate human anti-immunization peptide T cells when presented as a human lymphocyte antigen (HLA)-complex. Also preferably, a HPV16-derived peptide consisting of the same amino acid sequence as the at least one immunization peptide has been verified to be presented by human HPV16-positive tumor cells. Thus, preferably, the immunization peptide is a peptide verified to induce activation of immune cells and to be presented on cancer cells infected with HPV16.

In a preferred embodiment, the immunization peptide consists of an amino acid sequence comprising at least one, preferably at least two, more preferably three, cysteine residue(s). Thus, the immunization peptide, in a preferred embodiment, is selected from the list consisting of SEQ ID NOs: 1, 3-5, 7-11, 17, 24-27, 29-31, 33, 35-44, 47, 49, 50, 53, 55, 56, 58-60, 62-66,68-70, 73-77, 79, 80, 82-85, 89, 90, 92, 99, 101, 103-106, 108, 113-117, 121, 122, 126-128, 132-135, 137-139, 141-143, and 146, more preferably consisting of SEQ ID NOs: 4, 31, 53, 55, 56, 58, 62-66, 68, 69, 74-77, 79, 83, 84, 89, 90, 106, 121, 128, 135, and 139, even more preferably consisting of SEQ ID NOs: 77, 83, 90, 106, and 139. In an also preferred embodiment, the immunization peptide consists of an amino acid sequence comprising at least one cysteine residue and is presented via HLA-A01, preferably is selected from the list consisting of SEQ ID NOs:1, 3-5, 7-10, and 132-135; in a further preferred embodiment, the immunization peptide consists of an amino acid sequence comprising at least one cysteine residue and is presented via HLA-A02, preferably is selected from the list consisting of SEQ ID NOs:11, 17, 24-27, 29-31, 33, 35-44, 47, 49, and 50; in a further preferred embodiment, the immunization peptide consists of an amino acid sequence comprising at least one cysteine residue and is presented via HLA-A03, preferably is selected from the list consisting of SEQ ID NOs:53, 55, 63, 66, 69, 70, 73, 75-77, 135, and 137; in a further preferred embodiment, the immunization peptide consists of an amino acid sequence comprising at least one cysteine residue and is presented via HLA-A11, preferably is selected from the list consisting of SEQ ID NOs:56, 58-60, 62, 64, 65, 68, 74, and 79; in a further preferred embodiment, the immunization peptide consists of an amino acid sequence comprising at least one cysteine residue and is presented via HLA-A24, preferably is selected from the list consisting of SEQ ID NOs:80, 82-85, 89, 90, 92, 138, and 139; in a further preferred embodiment, the immunization peptide consists of an amino acid sequence comprising at least one cysteine residue and is presented via HLA-B07, preferably is selected from the list consisting of SEQ ID NOs:25, 99, 101, 103-106, and 141-143; in a further preferred embodiment, the immunization peptide consists of an amino acid sequence comprising at least one cysteine residue and is presented via HLA-B15, preferably is selected from the list consisting of SEQ ID NOs:9, 63, 85, 101, 108, 113-117, 121, 122, 126-128, and 146. In a further preferred embodiment, the immunization peptide consists of an amino acid sequence comprising at least one cysteine residue and is a peptide verified herein to be presented on cancer cells infected with HPV16, i.e. preferably is a selected from the list consisting of SEQ ID NOs:1, 11, 17, 53, 55, 56, 80, 108, 113, and 114. Thus, in a preferred embodiment, the the present invention relates to a vaccine providing at least one discrete immunization peptide consisting of an amino acid sequence selected from at least one of the following: SEQ ID NOs: 1; SEQ ID NOs:11 or 17; SEQ ID NOs:53, 55, or 56; SEQ ID NOs: 80; and SEQ ID NOs: 113 or 114.

The term “vaccine” is understood by the skilled person to include each and every agent eliciting an immune response in a subject. Preferably, said immune response is a cellular immune response, more preferably a T cell response, even more preferably a CD8+ T cell response, most preferably a cytotoxic T cell response. As the skilled person understands, the immunization peptides as referred to herein are derived from HPV16 E6 or E7, which are oncogenic HPV polypeptides; in accordance, the vaccine preferably is a therapeutic vaccine. Preferably, the vaccine comprises at least one polynucleotide, preferably an mRNA, encoding at least one of said immunization peptide(s) and/or a fusion polypeptide(s) as described herein below, is a nanoemulsion comprising said immunization peptide(s) and/or fusion polypeptide(s), or is an antigen presenting cell (APC) or artificial APC presenting said peptide(s) and/or fusion polypeptide(s).

Preferably, the vaccine is a pharmaceutical composition, optionally comprising a carrier; preferably, the carrier is a pharmaceutically acceptable carrier. In addition, vaccine may also include other additional compounds, such as adjuvants, stabilizers and/or other compounds deemed appropriate by the skilled person, e.g. for galenic purposes. As referred to herein, the immunization peptide(s) as specified herein above, is the “active compound” of the vaccine, although further active compounds may be present. Vaccine compositions are preferably prepared in a manner well known in the pharmaceutical art. Formulations are to be adapted to the mode of administration, i.e. in the forms of tablets, capsules, suppositories, solutions, suspensions or the like. Dosage recommendations shall be indicated in the prescriber's or user's instructions in order to anticipate dose adjustments depending on the considered recipient.

Preferably, the vaccine further comprises an adjuvant. More preferably, the immunization peptide(s) and the adjuvant are comprised in a common mixture at administration. Thus, preferably, the immunization peptide and the adjuvant are mixed before administration. Preferably, the adjuvant comprises a T-helper-1 response eliciting substance, such as e.g. a TLR-3 agonist (e.g. polyinosinic:polycytidylic acid, (poly(I:C)) or any derivative thereof wherein said derivative preferably is a polyl:polyC12U or poly-ICLC (Hiltonol™). However, also other adjuvants known to the skilled person may be used.

The term “eliciting an immune response” is understood by the skilled person and includes an increase of the extent of at least one feature of an immune response of a human subject to a non-self antigen. Thus, the term includes initiating a de novo response as well as enhancing an existing immune response. Preferably, eliciting an immune response comprises administering a vaccine providing an immunization peptide as specified herein above to a subject. Thus, the immunization peptide preferably is presented via a major histocompatibility complex (MHC) class I molecule, preferably on a nucleated cell, more preferably on an antigen presenting cell and/or on a nucleated cell infected with a HPV16-related virus, e.g. cells of an HPV16-related virus-positive inappropriate cellular proliferation, in particular a HPV-related lesion or HPV-related cancer as specified herein above. Thus, eliciting an immune response preferably is eliciting an immune response to a HPV16-related virus-positive inappropriate cellular proliferation. As usual in the art, MHCs may also be referred to as human lymphocyte antigens (HLAs), of which HLA-A, -B, and -C are MHC class I types. Preferably, eliciting an immune response comprises presentation of the immunization peptide on at least one HLA molecule belonging to one of HLA supertypes A1, A2, A3/A11, A24, B7, and B15.

Eliciting an immune response as referred to herein may comprise administration of a vaccine providing an immunization peptide to a subject as specified herein below, which proceeding may also be referred to as vaccination, in particular vaccination against HPV16-related virus infection. The term “vaccination” and in particular the term “vaccination against HPV16-related virus infection” as used herein, preferably, relates to administering a vaccine as specified herein to elicit an immune response against HPV16-related virus genotypes, in particular directed against cells infected therewith. Thus, vaccination preferably stimulates the immune system and establishes or improves immunity to infection with various HPV16-related virus genotypes. Preferably, vaccination according to the present invention allows for establishing or improving immunity to infection with HPV16-related virus genotypes. It is to be understood that the vaccine according to the present invention may comprise further components, in particular as specified elsewhere herein. The skilled person will understand that vaccination may not elicit a significant immune response in all subjects vaccinated. Also, it is to be understood that vaccination may not be effective to treat or prevent infection in all subjects vaccinated. However, the term requires that a, preferably statistically significant, portion of subjects of a cohort or population are effectively vaccinated, wherein effective vaccination, preferably, is prevention or reduction of the number of HPV16-related virus-induced lesions. Whether a portion is statistically significant can be determined without further ado by the person skilled in the art using various well known statistic evaluation tools, e.g., determination of confidence intervals, p-value determination, Student's t-test, Mann-Whitney test etc. Preferred confidence intervals are at least 90%, at least 95%, at least 97%, at least 98% or at least 99%. The p-values are, preferably, 0.1, 0.05, 0.01, 0.005, or 0.0001. Preferably, the treatment shall be effective for at least 60%, at least 70%, at least 80%, or at least 90% of the subjects of a given cohort or population. The aforesaid vaccination may be prophylactic, i.e. vaccination in a narrow sense, and provide preventing or reducing infection and/or ameliorating consequences of infection; or may be therapeutic, i.e. provide reduction or clearance of existing infection. Thus, in the context of HPV16-related virus-positive inappropriate cellular proliferations, therapeutic vaccination may in particular cause a reduction in number or complete clearance of cells infected with and/or transformed by said HPV16-related virus. In accordance, eliciting an immune response to a HPV16-related virus is eliciting an immune response to a HPV16-related virus infection, in particular to HPV16 E6 and/or E7 proteins.

Eliciting an immune response may, however, also relate to in vitro activating and/or enriching immune cells recognizing and/or binding said immunization peptide and administering said immune cells to said subject. As indicated herein above, said immune cells are T cells, preferably CD8+ T cells, more preferably cytotoxic CD8+ T cells. Also preferably, said immune cells may be antigen presenting cells loaded in vitro with at least one immunization peptide; also, synthetic APCs or scaffolds mimicking the same may be used. Thus, eliciting an immune response may comprise immune cell therapy. Preferably, said immune cells are HLA-matched to the cells of said subject, preferably are autologous immune cells.

The term “vaccine providing an immunization peptide”, as referred to herein, relates to any composition of matter causing an immunization peptide to become present, in particular after its application to a cell, preferably to a subject. Preferably, providing is causing an immunization peptide to become present by processes occurring naturally in a host cell, in particular proteasome proteolysis and/or MHC class I presentation pathways. Thus, the vaccine providing an immunization peptide may be any peptide or polypeptide comprising an immunization peptide; however, the vaccine providing an immunization peptide comprises at most 100, preferably at most 50, more preferably at most 25, most preferably at most 12, contiguous amino acids of a sequence of an HPV polypeptide. Preferably, the immunization peptide is linked in said vaccine to at least one of a mediator of HLA presentation, a linker, a solubility mediator, a cell-penetrating peptide, a detectable tag, a mediator of secretion, and/or an immune stimulant, or is an amphiphilic conjugate thereof, all of which are in principle known to the skilled person. From such a vaccine providing an immunization peptide, the immunization peptide may be liberated, e.g. by proteolysis (e.g. by a proteasome), by hydrolysis, e.g. of an amido or ester bond to a carrier molecule, and/or by fusion of a lipid vesicle, e.g. of a nanoemulsion, with a cell membrane. Corresponding compositions and methods are known in the art; as the skilled person is aware of, in case the immunization peptide is produced as part of a polypeptide, care is preferably taken to not create neoepitopes and/or interferences causing improper proteolysis. Thus, in such case, the immunization peptide is preferably flanked by appropriate linker sequences to avoid the aforesaid problems. Appropriate linker sequences are known in the art. Preferably, the vaccine providing an immunization peptide comprises an immunization peptide with an N-terminal cysteine coupled to (1,2-distearoyl-3-sn-phosphatidylethanolamine)-PEG-maleimide, as disclosed in Kruse et al. (2019), Oncolmmunology, 8(1):e1524694. Also preferably, the vaccine providing an immunization peptide is a nanoemulsion comprising at least one immunization peptide; corresponding compositions are known, e.g. from WO 2021/229015 A1, WO 2021/229014 A1, and WO 2021/229020 A1. Also preferably, the vaccine providing an immunization peptide may be a composition of matter comprising at least one immunization peptide as such. Preferably, the immunization peptide is presented by a host cell via major histocompatibility complex (MHC) class I.

The vaccine providing an immunization peptide may, however, also be a vaccine causing a host cell to synthesize an immunization peptide or a polypeptide comprising the same; for the peptides and polypeptides which may be produced from such a vaccine, reference is made to the description herein above. A corresponding vaccine providing an immunization peptide may in particular be a polynucleotide encoding at least one immunization peptide or a polypeptide comprising the same, preferably a polynucleotide encoding at least one immunization peptide. As the skilled person understands, more than one immunization peptide may be encoded by one polynucleotide, e.g. via a multivalent polypeptide, e.g. as a fusion polypeptide. Preferably, any HPV16-derived amino acid sequence comprised in a fusion polypeptide consists of at most 12 contiguous HPV16-derived amino acids, and/or each two of said immunization peptides in said fusion polypeptide are intervened by a non-HPV-derived amino acid sequence, preferably of at least 4, more preferably at least 5, still more preferably at least 6, even more preferably at least 7, most preferably at least 10, amino acids. Thus, the multivalent polypeptide preferably is not the full-length E6 polypeptide or the full-length E7 polypeptide. More preferably, however, a multitude of immunization peptides is encoded by separate coding sequences e.g. intervened by ribosomal reentry sites or encoded on separate polynucleotides. The polynucleotide may be any polynucleotide deemed appropriate by the skilled person for the intended use, taking into account e.g. mode of administration, target cell, required dose and duration of expression, and the like. Thus, the vaccine providing an immunization peptide may comprise an mRNA, an expression construct, optionally comprised in a vector, and the like. Thus, the vaccine providing an immunization peptide preferably is an mRNA or a DNA, preferably double-stranded DNA.

Preferably, the vaccine providing an immunization peptide provides a multitude of immunization peptides, thus, preferably, the vaccine providing an immunization peptide provides at least four, preferably at least five, most preferably at least six, of said immunization peptides, of which preferably at least two, preferably at least three, more preferably at least four, even more preferably at least five, most preferably at least six, are non-identical. Preferably, the vaccine providing an immunization peptide provides at least four, preferably at least five, most preferably at least six, non-identical immunization peptides binding to non-identical HLA supertypes selected from the list consisting of HLA supertypes A1, A2, A3/A11, A24, B7, and B15, wherein the term “A3/A11” relates to a common supertype of HLA-A3 and HLA-A11 types. The term “HLA supertypes” is known to the skilled person, e.g. from Sidney et al. (2008), BMC Immunology 9 Art. No. 1, doi.org/10.1186/1471-2172-9-1, where supertype B15, however, is referred to as B62. Also preferably, the vaccine providing an immunization peptide provides non-identical immunization peptides binding to at least five, preferably at least six, non-identical HLA supertypes. Preferably, the vaccine comprises a mixture of discrete peptides each providing exactly one of said immunization peptides and/or at least one polynucleotide providing said immunization peptides in a discrete manner.

The term “discrete”, in the context of the description herein, is understood by the skilled person in the sense of consisting of unconnected, distinct parts. Thus, distinct peptides are present in the vaccine as distinct parts; i.e. they are not comprised in a polypeptide comprising more than one of said peptides. In accordance, a polynucleotide providing immunization peptides in a discrete manner encodes said immunization peptides or peptides comprising immunization peptides separately, i.e. not as a fusion polypeptide. Appropriate methods are known in the art, e.g. by including ribosomal re-entry sites to a polynucleotide, enabling production of more than one peptide or polypeptide from e.g one RNA. As is understood by the skilled person in view of the description herein, at least one polynucleotide providing said immunization peptides in a discrete manner may also be a multitude of polynucleotides, each providing at least one immunization peptide in a discrete manner.

In view of the above, the vaccine preferably is a human leukocyte antigen (HLA) universal vaccine for use in any human subject expressing at least one HLA of a HLA supertype selected from the list consisting of HLA-A01, HLA-A02, HLA-A03/A11, HLA-A24, HLA-B07, and HLA-B15. The term “HLA universal vaccine”, as used herein, preferably relates to a vaccine providing at least one immunization peptide presented by a HLA in at least 90% of subjects of the world's population. Thus, the HLA universal vaccine preferably comprises a mixture of immunization peptides presented by HLA molecules of the most frequent HLA supertypes; frequencies of HLA supertypes are known in the art and are summarized herein below in Table 3.

The term “subject”, as used herein, relates to a mammal, preferably a human. Preferably the subject comprises at least one HLA type as specified herein below in Tables 1(i) to (vi). Preferably, the subject is infected with at least one HPV16-related virus. Preferably, the subject is afflicted with at least one HPV16-related virus positive inappropriate cellular proliferation.

Also preferably the HLA-supertypes expressed by said human subject are unknown. Also preferably, the subject is known or suspected to express at least one HLA from a HLA supertype selected from the list consisting of HLA-A01, HLA-A02, HLA-A3/A11, HLA-A24, HLA-B07, and HLA-B15. As will be understood by the skilled person from the disclosure herein, more than 90% of subjects in an average caucasoid, oriental, mixed, amerindian, african, or asian population, express at least one of the aforesaid HLA supertypes; as shown in Table 3. Thus, a subject preferably is suspected to express at least one HLA from one of said HLA supertypes unless there it is known, e.g. from HLA typing, not to do so.

Advantageously, it was found in the work underlying the present invention that HLA presentation of HPV16-derived peptides by HPV16-positive cells can be evaluated by mass spectrometry as disclosed in the Examples, providing experimental proof that peptides are in fact presented to the immune system on the intended target cells. Also, immunogenicity assays as shown in the Examples, performed on peripheral blood lymphocytes (PBMCs) of various donors can be used to provide proof that respective peptides are indeed active in vivo in inducing immune responses. Thus, in order to provide actionable target structures for vaccination against HPV16-mediated malignancies (therapeutic vaccines, adoptive transfer of T cells recognizing an immunization presented via a HLA on a target cell), it was found relevant to validate T cell epitope/HLA complexes as actionable targets to ensure efficacy of the above mentioned therapies. Preferably, it may not be sufficient to use HLA binding prediction algorithms to define actionable targets, as these are not precise enough and miss many binding peptides, cf. Table 2. Thus, not only HLA binding of each stimulation peptide referred to herein was experimentally validated, but, furthermore, stimulation peptides were assessed with PBMCs from healthy human donors for T cell memory responses. The thus defined immunogenic peptides must have been naturally processed and presented on activated APCs in a prior HPV16 infection. Also, it is preferred to validate the HLA presentation of putative stimulation peptides directly on the tumor (or other HPV-positive) cells. This can be achieved by a mass-spectrometry-based technology, immunopeptidomics. Thus, in particular the aforesaid two methods in combination can identify good candidate peptides for eliciting a cellular immune response against HPV16-related virus-infected cells and inappropriate cellular proliferations caused by them.

The present invention also relates to a vector comprising the HPV16 vaccine according to the present invention.

The term vector has been defined herein above. In accordance, the vector may in particular a polynucleotide adapted for expression and optionally maintenance in a host cell, preferably of a subject, said polynucleotide being a vaccine providing at least one discrete immunization peptide as specified herein above. Preferably, the vector comprises, alternatively or in addition to said polynucleotide, a polypeptide providing at least one discrete immunization peptide. Thus, in case the vector is a virus, it may comprise one or more viral structural polypeptide(s), e.g. a capsid polypeptide, comprising at least one immunization peptide.

The present invention further relates to a vaccine or vector of the present invention for use in medicine, and/or for use in eliciting an immune response in a human subject against an HPV16-related infection, wherein said eliciting an immune response preferably is treating and/or preventing HPV16-related inappropriate cellular proliferation.

The term “inappropriate cellular proliferation” relates to an abnormal proliferation of body cells in a subject; as a consequence, an imbalance of cellular composition of a body tissue, of a body fluid and/or tumor formation may ensue. Inappropriate cellular proliferation may be induced by an infectious agent, preferably a virus, more preferably an HPV16-related virus. Preferably, inappropriate cellular proliferation is benign, i.e. preferably, does not threaten health or life of a subject. Preferred benign inappropriate cellular proliferations are warts, exophytic growing papillomas, condylomata, inverted papillomas, and pre-neoplastic HPV-induced lesions. Also preferably, the inappropriate cellular proliferation is pre-malignant or malignant, i.e. does at least potentially threaten health or life of a subject; thus, preferably, the inappropriate cellular proliferation is a pre-malignant or malignant inappropriate proliferation of a mucosa or a skin, in particular a mucosa, e.g. of the oropharynx, anogenital regions, and/or reproductive organs, e.g. cervical intraepithelial neoplasia (CIN) or cancer, in particular cervical cancer and/or head and neck cancer. The term “cancer”, as used herein, relates to a disease of an animal, including man, characterized by uncontrolled growth by a group of body cells (“cancer cells”). This uncontrolled growth may be accompanied by intrusion into and destruction of surrounding tissue and possibly spread of cancer cells to other locations in the body. Preferably, also included by the term cancer is a relapse. Thus, preferably, the cancer is a solid cancer, a metastasis, or a relapse thereof. Thus, eliciting an immune response preferably is cancer prevention and/or cancer treatment.

The terms “treating” and “treatment” refer to an amelioration of the diseases or disorders referred to herein or the symptoms accompanied therewith to a significant extent. Said treating as used herein also includes an entire restoration of health with respect to the diseases or disorders referred to herein. It is to be understood that treating, as the term is used herein, may not be effective in all subjects to be treated. However, the term shall require that, preferably, a statistically significant portion of subjects suffering from a disease or disorder referred to herein can be successfully treated. Whether a portion is statistically significant can be determined without further ado by the person skilled in the art using various well known statistic evaluation tools, e.g., as described herein below. Preferably, treating cancer is reducing tumor burden in a subject. As will be understood by the skilled person, effectiveness of treatment of e.g. cancer is dependent on a variety of factors including, e.g. cancer stage and cancer type. Preferably, treating causes inappropriately proliferating cells, preferably neoplastic cells, more preferably cancer cells, to be recognized by T-cells of the subject. Thus, preferably, treating has the effect of killing tumor cells, causing a tumor to stop growing, in particular causing tumor cells to stop proliferating, more preferably causing regression of a tumor, more preferably causing a tumor to resolve. As used herein, the above relates to treating other HPV16 related virus-positive inappropriate cellular proliferations mutatis mutandis.

The term “preventing” refers to retaining health with respect to the diseases or disorders referred to herein for a certain period of time in a subject. It will be understood that the said period of time may be dependent on the amount of the drug compound which has been administered and individual factors of the subject discussed elsewhere in this specification. It is to be understood that prevention may not be effective in all subjects treated with the compound according to the present invention. However, the term requires that, preferably, a statistically significant portion of subjects of a cohort or population are effectively prevented from suffering from a disease or disorder referred to herein or its accompanying symptoms. Preferably, a cohort or population of subjects is envisaged in this context which normally, i.e. without preventive measures according to the present invention, would develop a disease or disorder as referred to herein. Whether a portion is statistically significant can be determined without further ado by the person skilled in the art using various well known statistic evaluation tools discussed elsewhere in this specification. Thus, preferably, in case of disease caused by an infectious agent, preventing may be vaccination. Thus, preferably, the term preventing relates to administering the compounds as specified herein to elicit an immune response against at least one infectious agent.

The present invention also relates to a vaccine for use in therapeutic vaccination of a human subject against a human papillomavirus 16 (HPV16)-related virus, wherein said vaccine provides at least one discrete immunization peptide consisting of an amino acid sequence selected from at least one of the following groups: SEQ ID NOs: 1 to 10; SEQ ID NOs:11 to 52; SEQ ID NO:53 to 79; SEQ ID NOs: 71 and 80 to 96; SEQ ID NOs:25, 81, and 97-106; and SEQ ID NO: 9, 12, 23, 63, 81, 85, 101, and 107 to 131.

The present invention also relates to a vaccine for use in therapeutic vaccination of a human subject against a human papillomavirus 16 (HPV16)-related virus, wherein said vaccine provides at least four discrete immunization peptides consisting of amino acid sequences selected from at least four of the following groups: SEQ ID NOs: 1 to 10; SEQ ID NOs:11 to 52; SEQ ID NO:53 to 79; SEQ ID NOs: 71 and 80 to 96; SEQ ID NOs:25, 81, and 97-106; and SEQ ID NO: 9, 12, 23, 63, 81, 85, 101, and 107 to 131; and wherein said at least four immunization peptides are selected such that at least 90% of subjects of the world's population express at least one human lymphocyte antigen (HLA) supertype presenting at least one of said immunization peptides.

In view of the description herein above, said at least four immunization peptides, when presented as a HLA-complexes activate human anti-immunization peptide T cell, respectively. Also preferably, at least one of said at least four immunogenic peptides consists of an amino acid sequence selected from group (ii) or (iii). More preferably, the vaccine provides at least six discrete immunization peptides consisting of amino acid sequences selected from all six of said groups. More preferably, (i) in case the immunization peptide is selected from SEQ ID NOs:1 to 10, the HLA supertype is HLA-A01; (ii) in case the immunization peptide is selected from SEQ ID NOs:11 to 52, the HLA supertype is HLA-A02; (iii) in case the immunization peptide is selected from SEQ ID NO:53 to 79, the HLA supertype is HLA-A03/A11; (iv) in case the immunization peptide is selected from SEQ ID NOs: 71 and 80 to 96, the HLA supertype is HLA-A24; (v) in case the immunization peptide is selected from SEQ ID NOs:25, 81, and 97-106, the HLA supertype is HLA-B07; and (vi) in case the immunization peptide is selected from SEQ ID NO: 9, 12, 23, 63, 81, 85, 101, and 107 to 131, the HLA supertype is HLA-B15.

Still more preferably, a HPV16-derived peptide consisting of the same amino acid sequence as at least one of said immunization peptides is presented by HPV16-related virus-positive cancer cells and wherein said at least one immunization peptide is selected from at least one of the following groups: SEQ ID NOs: 1 to 2; SEQ ID NOs:11 to 23; SEQ ID NO:53 to 57; SEQ ID NOs: 80 to 81; SEQ ID NOs: 81, 97, and 98 to; and SEQ ID NO: 81, and 107-114.

Preferably, the immunization peptides in the vaccine are selected from the immunization peptides found to have the highest rate of immunoactivation in PBMC samples, induce the strongest CD8 T cell response, and could be shown to be presented as HLA-complexes by HPV16 positive cells, so are preferably selected from (i) SEQ ID NOs:1 and 2, (ii) SEQ ID NOs: 11, 13, 14, 17, and 18, (iii) SEQ ID NO: 53, 54, and 55, (iv) SEQ ID NOs:80, 81, and 82; (v) SEQ ID NOs: 97, and 98, or SEQ ID NOs: 107, 108, and 109. More preferably, the vaccine provides at least six discrete immunization peptides consisting of amino acid sequences selected from all six of the aforesaid groups. Thus, preferably, the vaccine provides combinations of at least six discrete immunization peptides comprising SEQ ID NO 1, more preferably provides immunization polypeptides consisting of amino acids of the SEQ ID NOs: 1, 11, 53, 80, 81 and 107; 1, 11, 53, 80, 81 and 108; 1, 11, 53, 80, 81 and 109; 1, 11, 53, 80, 97 and 107; 1, 11, 53, 80, 97 and 108; 1, 11, 53, 80, 97 and 109; 1, 11, 53, 80, 98 and 107; 1, 11, 53, 80, 98 and 108; 1, 11, 53, 80, 98 and 109; 1, 11, 53, 81, 97 and 107; 1, 11, 53, 81, 97 and 108; 1, 11, 53, 81, 97 and 109; 1, 11, 53, 81, 98 and 107; 1, 11, 53, 81, 98 and 108; 1, 11, 53, 81, 98 and 109; 1, 11, 53, 82, 81 and 107; 1, 11, 53, 82, 81 and 108; 1, 11, 53, 82, 81 and 109; 1, 11, 53, 82, 97 and 107; 1, 11, 53, 82, 97 and 108; 1, 11, 53, 82, 97 and 109; 1, 11, 53, 82, 98 and 107; 1, 11, 53, 82, 98 and 108; 1, 11, 53, 82, 98 and 109; 1, 11, 54, 80, 81 and 107; 1, 11, 54, 80, 81 and 108; 1, 11, 54, 80, 81 and 109; 1, 11, 54, 80, 97 and 107; 1, 11, 54, 80, 97 and 108; 1, 11, 54, 80, 97 and 109; 1, 11, 54, 80, 98 and 107; 1, 11, 54, 80, 98 and 108; 1, 11, 54, 80, 98 and 109; 1, 11, 54, 81, 97 and 107; 1, 11, 54, 81, 97 and 108; 1, 11, 54, 81, 97 and 109; 1, 11, 54, 81, 98 and 107; 1, 11, 54, 81, 98 and 108; 1, 11, 54, 81, 98 and 109; 1, 11, 54, 82, 81 and 107; 1, 11, 54, 82, 81 and 108; 1, 11, 54, 82, 81 and 109; 1, 11, 54, 82, 97 and 107; 1, 11, 54, 82, 97 and 108; 1, 11, 54, 82, 97 and 109; 1, 11, 54, 82, 98 and 107; 1, 11, 54, 82, 98 and 108; 1, 11, 54, 82, 98 and 109; 1, 11, 55, 80, 81 and 107; 1, 11, 55, 80, 81 and 108; 1, 11, 55, 80, 81 and 109; 1, 11, 55, 80, 97 and 107; 1, 11, 55, 80, 97 and 108; 1, 11, 55, 80, 97 and 109; 1, 11, 55, 80, 98 and 107; 1, 11, 55, 80, 98 and 108; 1, 11, 55, 80, 98 and 109; 1, 11, 55, 81, 97 and 107; 1, 11, 55, 81, 97 and 108; 1, 11, 55, 81, 97 and 109; 1, 11, 55, 81, 98 and 107; 1, 11, 55, 81, 98 and 108; 1, 11, 55, 81, 98 and 109; 1, 11, 55, 82, 81 and 107; 1, 11, 55, 82, 81 and 108; 1, 11, 55, 82, 81 and 109; 1, 11, 55, 82, 97 and 107; 1, 11, 55, 82, 97 and 108; 1, 11, 55, 82, 97 and 109; 1, 11, 55, 82, 98 and 107; 1, 11, 55, 82, 98 and 108; 1, 11, 55, 82, 98 and 109; 1, 13, 53, 80, 81 and 107; 1, 13, 53, 80, 81 and 108; 1, 13, 53, 80, 81 and 109; 1, 13, 53, 80, 97 and 107; 1, 13, 53, 80, 97 and 108; 1, 13, 53, 80, 97 and 109; 1, 13, 53, 80, 98 and 107; 1, 13, 53, 80, 98 and 108; 1, 13, 53, 80, 98 and 109; 1, 13, 53, 81, 97 and 107; 1, 13, 53, 81, 97 and 108; 1, 13, 53, 81, 97 and 109; 1, 13, 53, 81, 98 and 107; 1, 13, 53, 81, 98 and 108; 1, 13, 53, 81, 98 and 109; 1, 13, 53, 82, 81 and 107; 1, 13, 53, 82, 81 and 108; 1, 13, 53, 82, 81 and 109; 1, 13, 53, 82, 97 and 107; 1, 13, 53, 82, 97 and 108; 1, 13, 53, 82, 97 and 109; 1, 13, 53, 82, 98 and 107; 1, 13, 53, 82, 98 and 108; 1, 13, 53, 82, 98 and 109; 1, 13, 54, 80, 81 and 107; 1, 13, 54, 80, 81 and 108; 1, 13, 54, 80, 81 and 109; 1, 13, 54, 80, 97 and 107; 1, 13, 54, 80, 97 and 108; 1, 13, 54, 80, 97 and 109; 1, 13, 54, 80, 98 and 107; 1, 13, 54, 80, 98 and 108; 1, 13, 54, 80, 98 and 109; 1, 13, 54, 81, 97 and 107; 1, 13, 54, 81, 97 and 108; 1, 13, 54, 81, 97 and 109; 1, 13, 54, 81, 98 and 107; 1, 13, 54, 81, 98 and 108; 1, 13, 54, 81, 98 and 109; 1, 13, 54, 82, 81 and 107; 1, 13, 54, 82, 81 and 108; 1, 13, 54, 82, 81 and 109; 1, 13, 54, 82, 97 and 107; 1, 13, 54, 82, 97 and 108; 1, 13, 54, 82, 97 and 109; 1, 13, 54, 82, 98 and 107; 1, 13, 54, 82, 98 and 108; 1, 13, 54, 82, 98 and 109; 1, 13, 55, 80, 81 and 107; 1, 13, 55, 80, 81 and 108; 1, 13, 55, 80, 81 and 109; 1, 13, 55, 80, 97 and 107; 1, 13, 55, 80, 97 and 108; 1, 13, 55, 80, 97 and 109; 1, 13, 55, 80, 98 and 107; 1, 13, 55, 80, 98 and 108; 1, 13, 55, 80, 98 and 109; 1, 13, 55, 81, 97 and 107; 1, 13, 55, 81, 97 and 108; 1, 13, 55, 81, 97 and 109; 1, 13, 55, 81, 98 and 107; 1, 13, 55, 81, 98 and 108; 1, 13, 55, 81, 98 and 109; 1, 13, 55, 82, 81 and 107; 1, 13, 55, 82, 81 and 108; 1, 13, 55, 82, 81 and 109; 1, 13, 55, 82, 97 and 107; 1, 13, 55, 82, 97 and 108; 1, 13, 55, 82, 97 and 109; 1, 13, 55, 82, 98 and 107; 1, 13, 55, 82, 98 and 108; 1, 13, 55, 82, 98 and 109; 1, 14, 53, 80, 81 and 107; 1, 14, 53, 80, 81 and 108; 1, 14, 53, 80, 81 and 109; 1, 14, 53, 80, 97 and 107; 1, 14, 53, 80, 97 and 108; 1, 14, 53, 80, 97 and 109; 1, 14, 53, 80, 98 and 107; 1, 14, 53, 80, 98 and 108; 1, 14, 53, 80, 98 and 109; 1, 14, 53, 81, 97 and 107; 1, 14, 53, 81, 97 and 108; 1, 14, 53, 81, 97 and 109; 1, 14, 53, 81, 98 and 107; 1, 14, 53, 81, 98 and 108; 1, 14, 53, 81, 98 and 109; 1, 14, 53, 82, 81 and 107; 1, 14, 53, 82, 81 and 108; 1, 14, 53, 82, 81 and 109; 1, 14, 53, 82, 97 and 107; 1, 14, 53, 82, 97 and 108; 1, 14, 53, 82, 97 and 109; 1, 14, 53, 82, 98 and 107; 1, 14, 53, 82, 98 and 108; 1, 14, 53, 82, 98 and 109; 1, 14, 54, 80, 81 and 107; 1, 14, 54, 80, 81 and 108; 1, 14, 54, 80, 81 and 109; 1, 14, 54, 80, 97 and 107; 1, 14, 54, 80, 97 and 108; 1, 14, 54, 80, 97 and 109; 1, 14, 54, 80, 98 and 107; 1, 14, 54, 80, 98 and 108; 1, 14, 54, 80, 98 and 109; 1, 14, 54, 81, 97 and 107; 1, 14, 54, 81, 97 and 108; 1, 14, 54, 81, 97 and 109; 1, 14, 54, 81, 98 and 107; 1, 14, 54, 81, 98 and 108; 1, 14, 54, 81, 98 and 109; 1, 14, 54, 82, 81 and 107; 1, 14, 54, 82, 81 and 108; 1, 14, 54, 82, 81 and 109; 1, 14, 54, 82, 97 and 107; 1, 14, 54, 82, 97 and 108; 1, 14, 54, 82, 97 and 109; 1, 14, 54, 82, 98 and 107; 1, 14, 54, 82, 98 and 108; 1, 14, 54, 82, 98 and 109; 1, 14, 55, 80, 81 and 107; 1, 14, 55, 80, 81 and 108; 1, 14, 55, 80, 81 and 109; 1, 14, 55, 80, 97 and 107; 1, 14, 55, 80, 97 and 108; 1, 14, 55, 80, 97 and 109; 1, 14, 55, 80, 98 and 107; 1, 14, 55, 80, 98 and 108; 1, 14, 55, 80, 98 and 109; 1, 14, 55, 81, 97 and 107; 1, 14, 55, 81, 97 and 108; 1, 14, 55, 81, 97 and 109; 1, 14, 55, 81, 98 and 107; 1, 14, 55, 81, 98 and 108; 1, 14, 55, 81, 98 and 109; 1, 14, 55, 82, 81 and 107; 1, 14, 55, 82, 81 and 108; 1, 14, 55, 82, 81 and 109; 1, 14, 55, 82, 97 and 107; 1, 14, 55, 82, 97 and 108; 1, 14, 55, 82, 97 and 109; 1, 14, 55, 82, 98 and 107; 1, 14, 55, 82, 98 and 108; 1, 14, 55, 82, 98 and 109; 1, 17, 53, 80, 81 and 107; 1, 17, 53, 80, 81 and 108; 1, 17, 53, 80, 81 and 109; 1, 17, 53, 80, 97 and 107; 1, 17, 53, 80, 97 and 108; 1, 17, 53, 80, 97 and 109; 1, 17, 53, 80, 98 and 107; 1, 17, 53, 80, 98 and 108; 1, 17, 53, 80, 98 and 109; 1, 17, 53, 81, 97 and 107; 1, 17, 53, 81, 97 and 108; 1, 17, 53, 81, 97 and 109; 1, 17, 53, 81, 98 and 107; 1, 17, 53, 81, 98 and 108; 1, 17, 53, 81, 98 and 109; 1, 17, 53, 82, 81 and 107; 1, 17, 53, 82, 81 and 108; 1, 17, 53, 82, 81 and 109; 1, 17, 53, 82, 97 and 107; 1, 17, 53, 82, 97 and 108; 1, 17, 53, 82, 97 and 109; 1, 17, 53, 82, 98 and 107; 1, 17, 53, 82, 98 and 108; 1, 17, 53, 82, 98 and 109; 1, 17, 54, 80, 81 and 107; 1, 17, 54, 80, 81 and 108; 1, 17, 54, 80, 81 and 109; 1, 17, 54, 80, 97 and 107; 1, 17, 54, 80, 97 and 108; 1, 17, 54, 80, 97 and 109; 1, 17, 54, 80, 98 and 107; 1, 17, 54, 80, 98 and 108; 1, 17, 54, 80, 98 and 109; 1, 17, 54, 81, 97 and 107; 1, 17, 54, 81, 97 and 108; 1, 17, 54, 81, 97 and 109; 1, 17, 54, 81, 98 and 107; 1, 17, 54, 81, 98 and 108; 1, 17, 54, 81, 98 and 109; 1, 17, 54, 82, 81 and 107; 1, 17, 54, 82, 81 and 108; 1, 17, 54, 82, 81 and 109; 1, 17, 54, 82, 97 and 107; 1, 17, 54, 82, 97 and 108; 1, 17, 54, 82, 97 and 109; 1, 17, 54, 82, 98 and 107; 1, 17, 54, 82, 98 and 108; 1, 17, 54, 82, 98 and 109; 1, 17, 55, 80, 81 and 107; 1, 17, 55, 80, 81 and 108; 1, 17, 55, 80, 81 and 109; 1, 17, 55, 80, 97 and 107; 1, 17, 55, 80, 97 and 108; 1, 17, 55, 80, 97 and 109; 1, 17, 55, 80, 98 and 107; 1, 17, 55, 80, 98 and 108; 1, 17, 55, 80, 98 and 109; 1, 17, 55, 81, 97 and 107; 1, 17, 55, 81, 97 and 108; 1, 17, 55, 81, 97 and 109; 1, 17, 55, 81, 98 and 107; 1, 17, 55, 81, 98 and 108; 1, 17, 55, 81, 98 and 109; 1, 17, 55, 82, 81 and 107; 1, 17, 55, 82, 81 and 108; 1, 17, 55, 82, 81 and 109; 1, 17, 55, 82, 97 and 107; 1, 17, 55, 82, 97 and 108; 1, 17, 55, 82, 97 and 109; 1, 17, 55, 82, 98 and 107; 1, 17, 55, 82, 98 and 108; 1, 17, 55, 82, 98 and 109; 1, 18, 53, 80, 81 and 107; 1, 18, 53, 80, 81 and 108; 1, 18, 53, 80, 81 and 109; 1, 18, 53, 80, 97 and 107; 1, 18, 53, 80, 97 and 108; 1, 18, 53, 80, 97 and 109; 1, 18, 53, 80, 98 and 107; 1, 18, 53, 80, 98 and 108; 1, 18, 53, 80, 98 and 109; 1, 18, 53, 81, 97 and 107; 1, 18, 53, 81, 97 and 108; 1, 18, 53, 81, 97 and 109; 1, 18, 53, 81, 98 and 107; 1, 18, 53, 81, 98 and 108; 1, 18, 53, 81, 98 and 109; 1, 18, 53, 82, 81 and 107; 1, 18, 53, 82, 81 and 108; 1, 18, 53, 82, 81 and 109; 1, 18, 53, 82, 97 and 107; 1, 18, 53, 82, 97 and 108; 1, 18, 53, 82, 97 and 109; 1, 18, 53, 82, 98 and 107; 1, 18, 53, 82, 98 and 108; 1, 18, 53, 82, 98 and 109; 1, 18, 54, 80, 81 and 107; 1, 18, 54, 80, 81 and 108; 1, 18, 54, 80, 81 and 109; 1, 18, 54, 80, 97 and 107; 1, 18, 54, 80, 97 and 108; 1, 18, 54, 80, 97 and 109; 1, 18, 54, 80, 98 and 107; 1, 18, 54, 80, 98 and 108; 1, 18, 54, 80, 98 and 109; 1, 18, 54, 81, 97 and 107; 1, 18, 54, 81, 97 and 108; 1, 18, 54, 81, 97 and 109; 1, 18, 54, 81, 98 and 107; 1, 18, 54, 81, 98 and 108; 1, 18, 54, 81, 98 and 109; 1, 18, 54, 82, 81 and 107; 1, 18, 54, 82, 81 and 108; 1, 18, 54, 82, 81 and 109; 1, 18, 54, 82, 97 and 107; 1, 18, 54, 82, 97 and 108; 1, 18, 54, 82, 97 and 109; 1, 18, 54, 82, 98 and 107; 1, 18, 54, 82, 98 and 108; 1, 18, 54, 82, 98 and 109; 1, 18, 55, 80, 81 and 107; 1, 18, 55, 80, 81 and 108; 1, 18, 55, 80, 81 and 109; 1, 18, 55, 80, 97 and 107; 1, 18, 55, 80, 97 and 108; 1, 18, 55, 80, 97 and 109; 1, 18, 55, 80, 98 and 107; 1, 18, 55, 80, 98 and 108; 1, 18, 55, 80, 98 and 109; 1, 18, 55, 81, 97 and 107; 1, 18, 55, 81, 97 and 108; 1, 18, 55, 81, 97 and 109; 1, 18, 55, 81, 98 and 107; 1, 18, 55, 81, 98 and 108; 1, 18, 55, 81, 98 and 109; 1, 18, 55, 82, 81 and 107; 1, 18, 55, 82, 81 and 108; 1, 18, 55, 82, 81 and 109; 1, 18, 55, 82, 97 and 107; 1, 18, 55, 82, 97 and 108; 1, 18, 55, 82, 97 and 109; 1, 18, 55, 82, 98 and 107; 1, 18, 55, 82, 98 and 108; or 1, 18, 55, 82, 98 and 109.

Also preferably, the vaccine provides combinations of at least six discrete immunization peptides comprising SEQ ID NO:2, more preferably provides immunization polypeptides consisting of amino acids of SEQ ID NOs: 2, 11, 53, 80, 81 and 107; 2, 11, 53, 80, 81 and 108; 2, 11, 53, 80, 81 and 109; 2, 11, 53, 80, 97 and 107; 2, 11, 53, 80, 97 and 108; 2, 11, 53, 80, 97 and 109; 2, 11, 53, 80, 98 and 107; 2, 11, 53, 80, 98 and 108; 2, 11, 53, 80, 98 and 109; 2, 11, 53, 81, 97 and 107; 2, 11, 53, 81, 97 and 108; 2, 11, 53, 81, 97 and 109; 2, 11, 53, 81, 98 and 107; 2, 11, 53, 81, 98 and 108; 2, 11, 53, 81, 98 and 109; 2, 11, 53, 82, 81 and 107; 2, 11, 53, 82, 81 and 108; 2, 11, 53, 82, 81 and 109; 2, 11, 53, 82, 97 and 107; 2, 11, 53, 82, 97 and 108; 2, 11, 53, 82, 97 and 109; 2, 11, 53, 82, 98 and 107; 2, 11, 53, 82, 98 and 108; 2, 11, 53, 82, 98 and 109; 2, 11, 54, 80, 81 and 107; 2, 11, 54, 80, 81 and 108; 2, 11, 54, 80, 81 and 109; 2, 11, 54, 80, 97 and 107; 2, 11, 54, 80, 97 and 108; 2, 11, 54, 80, 97 and 109; 2, 11, 54, 80, 98 and 107; 2, 11, 54, 80, 98 and 108; 2, 11, 54, 80, 98 and 109; 2, 11, 54, 81, 97 and 107; 2, 11, 54, 81, 97 and 108; 2, 11, 54, 81, 97 and 109; 2, 11, 54, 81, 98 and 107; 2, 11, 54, 81, 98 and 108; 2, 11, 54, 81, 98 and 109; 2, 11, 54, 82, 81 and 107; 2, 11, 54, 82, 81 and 108; 2, 11, 54, 82, 81 and 109; 2, 11, 54, 82, 97 and 107; 2, 11, 54, 82, 97 and 108; 2, 11, 54, 82, 97 and 109; 2, 11, 54, 82, 98 and 107; 2, 11, 54, 82, 98 and 108; 2, 11, 54, 82, 98 and 109; 2, 11, 55, 80, 81 and 107; 2, 11, 55, 80, 81 and 108; 2, 11, 55, 80, 81 and 109; 2, 11, 55, 80, 97 and 107; 2, 11, 55, 80, 97 and 108; 2, 11, 55, 80, 97 and 109; 2, 11, 55, 80, 98 and 107; 2, 11, 55, 80, 98 and 108; 2, 11, 55, 80, 98 and 109; 2, 11, 55, 81, 97 and 107; 2, 11, 55, 81, 97 and 108; 2, 11, 55, 81, 97 and 109; 2, 11, 55, 81, 98 and 107; 2, 11, 55, 81, 98 and 108; 2, 11, 55, 81, 98 and 109; 2, 11, 55, 82, 81 and 107; 2, 11, 55, 82, 81 and 108; 2, 11, 55, 82, 81 and 109; 2, 11, 55, 82, 97 and 107; 2, 11, 55, 82, 97 and 108; 2, 11, 55, 82, 97 and 109; 2, 11, 55, 82, 98 and 107; 2, 11, 55, 82, 98 and 108; 2, 11, 55, 82, 98 and 109; 2, 13, 53, 80, 81 and 107; 2, 13, 53, 80, 81 and 108; 2, 13, 53, 80, 81 and 109; 2, 13, 53, 80, 97 and 107; 2, 13, 53, 80, 97 and 108; 2, 13, 53, 80, 97 and 109; 2, 13, 53, 80, 98 and 107; 2, 13, 53, 80, 98 and 108; 2, 13, 53, 80, 98 and 109; 2, 13, 53, 81, 97 and 107; 2, 13, 53, 81, 97 and 108; 2, 13, 53, 81, 97 and 109; 2, 13, 53, 81, 98 and 107; 2, 13, 53, 81, 98 and 108; 2, 13, 53, 81, 98 and 109; 2, 13, 53, 82, 81 and 107; 2, 13, 53, 82, 81 and 108; 2, 13, 53, 82, 81 and 109; 2, 13, 53, 82, 97 and 107; 2, 13, 53, 82, 97 and 108; 2, 13, 53, 82, 97 and 109; 2, 13, 53, 82, 98 and 107; 2, 13, 53, 82, 98 and 108; 2, 13, 53, 82, 98 and 109; 2, 13, 54, 80, 81 and 107; 2, 13, 54, 80, 81 and 108; 2, 13, 54, 80, 81 and 109; 2, 13, 54, 80, 97 and 107; 2, 13, 54, 80, 97 and 108; 2, 13, 54, 80, 97 and 109; 2, 13, 54, 80, 98 and 107; 2, 13, 54, 80, 98 and 108; 2, 13, 54, 80, 98 and 109; 2, 13, 54, 81, 97 and 107; 2, 13, 54, 81, 97 and 108; 2, 13, 54, 81, 97 and 109; 2, 13, 54, 81, 98 and 107; 2, 13, 54, 81, 98 and 108; 2, 13, 54, 81, 98 and 109; 2, 13, 54, 82, 81 and 107; 2, 13, 54, 82, 81 and 108; 2, 13, 54, 82, 81 and 109; 2, 13, 54, 82, 97 and 107; 2, 13, 54, 82, 97 and 108; 2, 13, 54, 82, 97 and 109; 2, 13, 54, 82, 98 and 107; 2, 13, 54, 82, 98 and 108; 2, 13, 54, 82, 98 and 109; 2, 13, 55, 80, 81 and 107; 2, 13, 55, 80, 81 and 108; 2, 13, 55, 80, 81 and 109; 2, 13, 55, 80, 97 and 107; 2, 13, 55, 80, 97 and 108; 2, 13, 55, 80, 97 and 109; 2, 13, 55, 80, 98 and 107; 2, 13, 55, 80, 98 and 108; 2, 13, 55, 80, 98 and 109; 2, 13, 55, 81, 97 and 107; 2, 13, 55, 81, 97 and 108; 2, 13, 55, 81, 97 and 109; 2, 13, 55, 81, 98 and 107; 2, 13, 55, 81, 98 and 108; 2, 13, 55, 81, 98 and 109; 2, 13, 55, 82, 81 and 107; 2, 13, 55, 82, 81 and 108; 2, 13, 55, 82, 81 and 109; 2, 13, 55, 82, 97 and 107; 2, 13, 55, 82, 97 and 108; 2, 13, 55, 82, 97 and 109; 2, 13, 55, 82, 98 and 107; 2, 13, 55, 82, 98 and 108; 2, 13, 55, 82, 98 and 109; 2, 14, 53, 80, 81 and 107; 2, 14, 53, 80, 81 and 108; 2, 14, 53, 80, 81 and 109; 2, 14, 53, 80, 97 and 107; 2, 14, 53, 80, 97 and 108; 2, 14, 53, 80, 97 and 109; 2, 14, 53, 80, 98 and 107; 2, 14, 53, 80, 98 and 108; 2, 14, 53, 80, 98 and 109; 2, 14, 53, 81, 97 and 107; 2, 14, 53, 81, 97 and 108; 2, 14, 53, 81, 97 and 109; 2, 14, 53, 81, 98 and 107; 2, 14, 53, 81, 98 and 108; 2, 14, 53, 81, 98 and 109; 2, 14, 53, 82, 81 and 107; 2, 14, 53, 82, 81 and 108; 2, 14, 53, 82, 81 and 109; 2, 14, 53, 82, 97 and 107; 2, 14, 53, 82, 97 and 108; 2, 14, 53, 82, 97 and 109; 2, 14, 53, 82, 98 and 107; 2, 14, 53, 82, 98 and 108; 2, 14, 53, 82, 98 and 109; 2, 14, 54, 80, 81 and 107; 2, 14, 54, 80, 81 and 108; 2, 14, 54, 80, 81 and 109; 2, 14, 54, 80, 97 and 107; 2, 14, 54, 80, 97 and 108; 2, 14, 54, 80, 97 and 109; 2, 14, 54, 80, 98 and 107; 2, 14, 54, 80, 98 and 108; 2, 14, 54, 80, 98 and 109; 2, 14, 54, 81, 97 and 107; 2, 14, 54, 81, 97 and 108; 2, 14, 54, 81, 97 and 109; 2, 14, 54, 81, 98 and 107; 2, 14, 54, 81, 98 and 108; 2, 14, 54, 81, 98 and 109; 2, 14, 54, 82, 81 and 107; 2, 14, 54, 82, 81 and 108; 2, 14, 54, 82, 81 and 109; 2, 14, 54, 82, 97 and 107; 2, 14, 54, 82, 97 and 108; 2, 14, 54, 82, 97 and 109; 2, 14, 54, 82, 98 and 107; 2, 14, 54, 82, 98 and 108; 2, 14, 54, 82, 98 and 109; 2, 14, 55, 80, 81 and 107; 2, 14, 55, 80, 81 and 108; 2, 14, 55, 80, 81 and 109; 2, 14, 55, 80, 97 and 107; 2, 14, 55, 80, 97 and 108; 2, 14, 55, 80, 97 and 109; 2, 14, 55, 80, 98 and 107; 2, 14, 55, 80, 98 and 108; 2, 14, 55, 80, 98 and 109; 2, 14, 55, 81, 97 and 107; 2, 14, 55, 81, 97 and 108; 2, 14, 55, 81, 97 and 109; 2, 14, 55, 81, 98 and 107; 2, 14, 55, 81, 98 and 108; 2, 14, 55, 81, 98 and 109; 2, 14, 55, 82, 81 and 107; 2, 14, 55, 82, 81 and 108; 2, 14, 55, 82, 81 and 109; 2, 14, 55, 82, 97 and 107; 2, 14, 55, 82, 97 and 108; 2, 14, 55, 82, 97 and 109; 2, 14, 55, 82, 98 and 107; 2, 14, 55, 82, 98 and 108; 2, 14, 55, 82, 98 and 109; 2, 17, 53, 80, 81 and 107; 2, 17, 53, 80, 81 and 108; 2, 17, 53, 80, 81 and 109; 2, 17, 53, 80, 97 and 107; 2, 17, 53, 80, 97 and 108; 2, 17, 53, 80, 97 and 109; 2, 17, 53, 80, 98 and 107; 2, 17, 53, 80, 98 and 108; 2, 17, 53, 80, 98 and 109; 2, 17, 53, 81, 97 and 107; 2, 17, 53, 81, 97 and 108; 2, 17, 53, 81, 97 and 109; 2, 17, 53, 81, 98 and 107; 2, 17, 53, 81, 98 and 108; 2, 17, 53, 81, 98 and 109; 2, 17, 53, 82, 81 and 107; 2, 17, 53, 82, 81 and 108; 2, 17, 53, 82, 81 and 109; 2, 17, 53, 82, 97 and 107; 2, 17, 53, 82, 97 and 108; 2, 17, 53, 82, 97 and 109; 2, 17, 53, 82, 98 and 107; 2, 17, 53, 82, 98 and 108; 2, 17, 53, 82, 98 and 109; 2, 17, 54, 80, 81 and 107; 2, 17, 54, 80, 81 and 108; 2, 17, 54, 80, 81 and 109; 2, 17, 54, 80, 97 and 107; 2, 17, 54, 80, 97 and 108; 2, 17, 54, 80, 97 and 109; 2, 17, 54, 80, 98 and 107; 2, 17, 54, 80, 98 and 108; 2, 17, 54, 80, 98 and 109; 2, 17, 54, 81, 97 and 107; 2, 17, 54, 81, 97 and 108; 2, 17, 54, 81, 97 and 109; 2, 17, 54, 81, 98 and 107; 2, 17, 54, 81, 98 and 108; 2, 17, 54, 81, 98 and 109; 2, 17, 54, 82, 81 and 107; 2, 17, 54, 82, 81 and 108; 2, 17, 54, 82, 81 and 109; 2, 17, 54, 82, 97 and 107; 2, 17, 54, 82, 97 and 108; 2, 17, 54, 82, 97 and 109; 2, 17, 54, 82, 98 and 107; 2, 17, 54, 82, 98 and 108; 2, 17, 54, 82, 98 and 109; 2, 17, 55, 80, 81 and 107; 2, 17, 55, 80, 81 and 108; 2, 17, 55, 80, 81 and 109; 2, 17, 55, 80, 97 and 107; 2, 17, 55, 80, 97 and 108; 2, 17, 55, 80, 97 and 109; 2, 17, 55, 80, 98 and 107; 2, 17, 55, 80, 98 and 108; 2, 17, 55, 80, 98 and 109; 2, 17, 55, 81, 97 and 107; 2, 17, 55, 81, 97 and 108; 2, 17, 55, 81, 97 and 109; 2, 17, 55, 81, 98 and 107; 2, 17, 55, 81, 98 and 108; 2, 17, 55, 81, 98 and 109; 2, 17, 55, 82, 81 and 107; 2, 17, 55, 82, 81 and 108; 2, 17, 55, 82, 81 and 109; 2, 17, 55, 82, 97 and 107; 2, 17, 55, 82, 97 and 108; 2, 17, 55, 82, 97 and 109; 2, 17, 55, 82, 98 and 107; 2, 17, 55, 82, 98 and 108; 2, 17, 55, 82, 98 and 109; 2, 18, 53, 80, 81 and 107; 2, 18, 53, 80, 81 and 108; 2, 18, 53, 80, 81 and 109; 2, 18, 53, 80, 97 and 107; 2, 18, 53, 80, 97 and 108; 2, 18, 53, 80, 97 and 109; 2, 18, 53, 80, 98 and 107; 2, 18, 53, 80, 98 and 108; 2, 18, 53, 80, 98 and 109; 2, 18, 53, 81, 97 and 107; 2, 18, 53, 81, 97 and 108; 2, 18, 53, 81, 97 and 109; 2, 18, 53, 81, 98 and 107; 2, 18, 53, 81, 98 and 108; 2, 18, 53, 81, 98 and 109; 2, 18, 53, 82, 81 and 107; 2, 18, 53, 82, 81 and 108; 2, 18, 53, 82, 81 and 109; 2, 18, 53, 82, 97 and 107; 2, 18, 53, 82, 97 and 108; 2, 18, 53, 82, 97 and 109; 2, 18, 53, 82, 98 and 107; 2, 18, 53, 82, 98 and 108; 2, 18, 53, 82, 98 and 109; 2, 18, 54, 80, 81 and 107; 2, 18, 54, 80, 81 and 108; 2, 18, 54, 80, 81 and 109; 2, 18, 54, 80, 97 and 107; 2, 18, 54, 80, 97 and 108; 2, 18, 54, 80, 97 and 109; 2, 18, 54, 80, 98 and 107; 2, 18, 54, 80, 98 and 108; 2, 18, 54, 80, 98 and 109; 2, 18, 54, 81, 97 and 107; 2, 18, 54, 81, 97 and 108; 2, 18, 54, 81, 97 and 109; 2, 18, 54, 81, 98 and 107; 2, 18, 54, 81, 98 and 108; 2, 18, 54, 81, 98 and 109; 2, 18, 54, 82, 81 and 107; 2, 18, 54, 82, 81 and 108; 2, 18, 54, 82, 81 and 109; 2, 18, 54, 82, 97 and 107; 2, 18, 54, 82, 97 and 108; 2, 18, 54, 82, 97 and 109; 2, 18, 54, 82, 98 and 107; 2, 18, 54, 82, 98 and 108; 2, 18, 54, 82, 98 and 109; 2, 18, 55, 80, 81 and 107; 2, 18, 55, 80, 81 and 108; 2, 18, 55, 80, 81 and 109; 2, 18, 55, 80, 97 and 107; 2, 18, 55, 80, 97 and 108; 2, 18, 55, 80, 97 and 109; 2, 18, 55, 80, 98 and 107; 2, 18, 55, 80, 98 and 108; 2, 18, 55, 80, 98 and 109; 2, 18, 55, 81, 97 and 107; 2, 18, 55, 81, 97 and 108; 2, 18, 55, 81, 97 and 109; 2, 18, 55, 81, 98 and 107; 2, 18, 55, 81, 98 and 108; 2, 18, 55, 81, 98 and 109; 2, 18, 55, 82, 81 and 107; 2, 18, 55, 82, 81 and 108; 2, 18, 55, 82, 81 and 109; 2, 18, 55, 82, 97 and 107; 2, 18, 55, 82, 97 and 108; 2, 18, 55, 82, 97 and 109; 2, 18, 55, 82, 98 and 107; 2, 18, 55, 82, 98 and 108; or 2, 18, 55, 82, 98 and 109.

Also preferably, the vaccine provides combinations of at least six discrete immunization peptides comprising SEQ ID NO:11, more preferably provides immunization polypeptides consisting of amino acids of SEQ ID NOs: 1, 11, 53, 80, 81 and 107; 1, 11, 53, 80, 81 and 108; 1, 11, 53, 80, 81 and 109; 1, 11, 53, 80, 97 and 107; 1, 11, 53, 80, 97 and 108; 1, 11, 53, 80, 97 and 109; 1, 11, 53, 80, 98 and 107; 1, 11, 53, 80, 98 and 108; 1, 11, 53, 80, 98 and 109; 1, 11, 53, 81, 97 and 107; 1, 11, 53, 81, 97 and 108; 1, 11, 53, 81, 97 and 109; 1, 11, 53, 81, 98 and 107; 1, 11, 53, 81, 98 and 108; 1, 11, 53, 81, 98 and 109; 1, 11, 53, 82, 81 and 107; 1, 11, 53, 82, 81 and 108; 1, 11, 53, 82, 81 and 109; 1, 11, 53, 82, 97 and 107; 1, 11, 53, 82, 97 and 108; 1, 11, 53, 82, 97 and 109; 1, 11, 53, 82, 98 and 107; 1, 11, 53, 82, 98 and 108; 1, 11, 53, 82, 98 and 109; 1, 11, 54, 80, 81 and 107; 1, 11, 54, 80, 81 and 108; 1, 11, 54, 80, 81 and 109; 1, 11, 54, 80, 97 and 107; 1, 11, 54, 80, 97 and 108; 1, 11, 54, 80, 97 and 109; 1, 11, 54, 80, 98 and 107; 1, 11, 54, 80, 98 and 108; 1, 11, 54, 80, 98 and 109; 1, 11, 54, 81, 97 and 107; 1, 11, 54, 81, 97 and 108; 1, 11, 54, 81, 97 and 109; 1, 11, 54, 81, 98 and 107; 1, 11, 54, 81, 98 and 108; 1, 11, 54, 81, 98 and 109; 1, 11, 54, 82, 81 and 107; 1, 11, 54, 82, 81 and 108; 1, 11, 54, 82, 81 and 109; 1, 11, 54, 82, 97 and 107; 1, 11, 54, 82, 97 and 108; 1, 11, 54, 82, 97 and 109; 1, 11, 54, 82, 98 and 107; 1, 11, 54, 82, 98 and 108; 1, 11, 54, 82, 98 and 109; 1, 11, 55, 80, 81 and 107; 1, 11, 55, 80, 81 and 108; 1, 11, 55, 80, 81 and 109; 1, 11, 55, 80, 97 and 107; 1, 11, 55, 80, 97 and 108; 1, 11, 55, 80, 97 and 109; 1, 11, 55, 80, 98 and 107; 1, 11, 55, 80, 98 and 108; 1, 11, 55, 80, 98 and 109; 1, 11, 55, 81, 97 and 107; 1, 11, 55, 81, 97 and 108; 1, 11, 55, 81, 97 and 109; 1, 11, 55, 81, 98 and 107; 1, 11, 55, 81, 98 and 108; 1, 11, 55, 81, 98 and 109; 1, 11, 55, 82, 81 and 107; 1, 11, 55, 82, 81 and 108; 1, 11, 55, 82, 81 and 109; 1, 11, 55, 82, 97 and 107; 1, 11, 55, 82, 97 and 108; 1, 11, 55, 82, 97 and 109; 1, 11, 55, 82, 98 and 107; 1, 11, 55, 82, 98 and 108; 1, 11, 55, 82, 98 and 109; 2, 11, 53, 80, 81 and 107; 2, 11, 53, 80, 81 and 108; 2, 11, 53, 80, 81 and 109; 2, 11, 53, 80, 97 and 107; 2, 11, 53, 80, 97 and 108; 2, 11, 53, 80, 97 and 109; 2, 11, 53, 80, 98 and 107; 2, 11, 53, 80, 98 and 108; 2, 11, 53, 80, 98 and 109; 2, 11, 53, 81, 97 and 107; 2, 11, 53, 81, 97 and 108; 2, 11, 53, 81, 97 and 109; 2, 11, 53, 81, 98 and 107; 2, 11, 53, 81, 98 and 108; 2, 11, 53, 81, 98 and 109; 2, 11, 53, 82, 81 and 107; 2, 11, 53, 82, 81 and 108; 2, 11, 53, 82, 81 and 109; 2, 11, 53, 82, 97 and 107; 2, 11, 53, 82, 97 and 108; 2, 11, 53, 82, 97 and 109; 2, 11, 53, 82, 98 and 107; 2, 11, 53, 82, 98 and 108; 2, 11, 53, 82, 98 and 109; 2, 11, 54, 80, 81 and 107; 2, 11, 54, 80, 81 and 108; 2, 11, 54, 80, 81 and 109; 2, 11, 54, 80, 97 and 107; 2, 11, 54, 80, 97 and 108; 2, 11, 54, 80, 97 and 109; 2, 11, 54, 80, 98 and 107; 2, 11, 54, 80, 98 and 108; 2, 11, 54, 80, 98 and 109; 2, 11, 54, 81, 97 and 107; 2, 11, 54, 81, 97 and 108; 2, 11, 54, 81, 97 and 109; 2, 11, 54, 81, 98 and 107; 2, 11, 54, 81, 98 and 108; 2, 11, 54, 81, 98 and 109; 2, 11, 54, 82, 81 and 107; 2, 11, 54, 82, 81 and 108; 2, 11, 54, 82, 81 and 109; 2, 11, 54, 82, 97 and 107; 2, 11, 54, 82, 97 and 108; 2, 11, 54, 82, 97 and 109; 2, 11, 54, 82, 98 and 107; 2, 11, 54, 82, 98 and 108; 2, 11, 54, 82, 98 and 109; 2, 11, 55, 80, 81 and 107; 2, 11, 55, 80, 81 and 108; 2, 11, 55, 80, 81 and 109; 2, 11, 55, 80, 97 and 107; 2, 11, 55, 80, 97 and 108; 2, 11, 55, 80, 97 and 109; 2, 11, 55, 80, 98 and 107; 2, 11, 55, 80, 98 and 108; 2, 11, 55, 80, 98 and 109; 2, 11, 55, 81, 97 and 107; 2, 11, 55, 81, 97 and 108; 2, 11, 55, 81, 97 and 109; 2, 11, 55, 81, 98 and 107; 2, 11, 55, 81, 98 and 108; 2, 11, 55, 81, 98 and 109; 2, 11, 55, 82, 81 and 107; 2, 11, 55, 82, 81 and 108; 2, 11, 55, 82, 81 and 109; 2, 11, 55, 82, 97 and 107; 2, 11, 55, 82, 97 and 108; 2, 11, 55, 82, 97 and 109; 2, 11, 55, 82, 98 and 107; 2, 11, 55, 82, 98 and 108; or 2, 11, 55, 82, 98 and 109.

Also preferably, the vaccine provides combinations of at least six discrete immunization peptides comprising SEQ ID NO:13, more preferably provides immunization polypeptides consisting of amino acids of SEQ ID NOs:1, 13, 53, 80, 81 and 107; 1, 13, 53, 80, 81 and 108; 1, 13, 53, 80, 81 and 109; 1, 13, 53, 80, 97 and 107; 1, 13, 53, 80, 97 and 108; 1, 13, 53, 80, 97 and 109; 1, 13, 53, 80, 98 and 107; 1, 13, 53, 80, 98 and 108; 1, 13, 53, 80, 98 and 109; 1, 13, 53, 81, 97 and 107; 1, 13, 53, 81, 97 and 108; 1, 13, 53, 81, 97 and 109; 1, 13, 53, 81, 98 and 107; 1, 13, 53, 81, 98 and 108; 1, 13, 53, 81, 98 and 109; 1, 13, 53, 82, 81 and 107; 1, 13, 53, 82, 81 and 108; 1, 13, 53, 82, 81 and 109; 1, 13, 53, 82, 97 and 107; 1, 13, 53, 82, 97 and 108; 1, 13, 53, 82, 97 and 109; 1, 13, 53, 82, 98 and 107; 1, 13, 53, 82, 98 and 108; 1, 13, 53, 82, 98 and 109; 1, 13, 54, 80, 81 and 107; 1, 13, 54, 80, 81 and 108; 1, 13, 54, 80, 81 and 109; 1, 13, 54, 80, 97 and 107; 1, 13, 54, 80, 97 and 108; 1, 13, 54, 80, 97 and 109; 1, 13, 54, 80, 98 and 107; 1, 13, 54, 80, 98 and 108; 1, 13, 54, 80, 98 and 109; 1, 13, 54, 81, 97 and 107; 1, 13, 54, 81, 97 and 108; 1, 13, 54, 81, 97 and 109; 1, 13, 54, 81, 98 and 107; 1, 13, 54, 81, 98 and 108; 1, 13, 54, 81, 98 and 109; 1, 13, 54, 82, 81 and 107; 1, 13, 54, 82, 81 and 108; 1, 13, 54, 82, 81 and 109; 1, 13, 54, 82, 97 and 107; 1, 13, 54, 82, 97 and 108; 1, 13, 54, 82, 97 and 109; 1, 13, 54, 82, 98 and 107; 1, 13, 54, 82, 98 and 108; 1, 13, 54, 82, 98 and 109; 1, 13, 55, 80, 81 and 107; 1, 13, 55, 80, 81 and 108; 1, 13, 55, 80, 81 and 109; 1, 13, 55, 80, 97 and 107; 1, 13, 55, 80, 97 and 108; 1, 13, 55, 80, 97 and 109; 1, 13, 55, 80, 98 and 107; 1, 13, 55, 80, 98 and 108; 1, 13, 55, 80, 98 and 109; 1, 13, 55, 81, 97 and 107; 1, 13, 55, 81, 97 and 108; 1, 13, 55, 81, 97 and 109; 1, 13, 55, 81, 98 and 107; 1, 13, 55, 81, 98 and 108; 1, 13, 55, 81, 98 and 109; 1, 13, 55, 82, 81 and 107; 1, 13, 55, 82, 81 and 108; 1, 13, 55, 82, 81 and 109; 1, 13, 55, 82, 97 and 107; 1, 13, 55, 82, 97 and 108; 1, 13, 55, 82, 97 and 109; 1, 13, 55, 82, 98 and 107; 1, 13, 55, 82, 98 and 108; 1, 13, 55, 82, 98 and 109; 2, 13, 53, 80, 81 and 107; 2, 13, 53, 80, 81 and 108; 2, 13, 53, 80, 81 and 109; 2, 13, 53, 80, 97 and 107; 2, 13, 53, 80, 97 and 108; 2, 13, 53, 80, 97 and 109; 2, 13, 53, 80, 98 and 107; 2, 13, 53, 80, 98 and 108; 2, 13, 53, 80, 98 and 109; 2, 13, 53, 81, 97 and 107; 2, 13, 53, 81, 97 and 108; 2, 13, 53, 81, 97 and 109; 2, 13, 53, 81, 98 and 107; 2, 13, 53, 81, 98 and 108; 2, 13, 53, 81, 98 and 109; 2, 13, 53, 82, 81 and 107; 2, 13, 53, 82, 81 and 108; 2, 13, 53, 82, 81 and 109; 2, 13, 53, 82, 97 and 107; 2, 13, 53, 82, 97 and 108; 2, 13, 53, 82, 97 and 109; 2, 13, 53, 82, 98 and 107; 2, 13, 53, 82, 98 and 108; 2, 13, 53, 82, 98 and 109; 2, 13, 54, 80, 81 and 107; 2, 13, 54, 80, 81 and 108; 2, 13, 54, 80, 81 and 109; 2, 13, 54, 80, 97 and 107; 2, 13, 54, 80, 97 and 108; 2, 13, 54, 80, 97 and 109; 2, 13, 54, 80, 98 and 107; 2, 13, 54, 80, 98 and 108; 2, 13, 54, 80, 98 and 109; 2, 13, 54, 81, 97 and 107; 2, 13, 54, 81, 97 and 108; 2, 13, 54, 81, 97 and 109; 2, 13, 54, 81, 98 and 107; 2, 13, 54, 81, 98 and 108; 2, 13, 54, 81, 98 and 109; 2, 13, 54, 82, 81 and 107; 2, 13, 54, 82, 81 and 108; 2, 13, 54, 82, 81 and 109; 2, 13, 54, 82, 97 and 107; 2, 13, 54, 82, 97 and 108; 2, 13, 54, 82, 97 and 109; 2, 13, 54, 82, 98 and 107; 2, 13, 54, 82, 98 and 108; 2, 13, 54, 82, 98 and 109; 2, 13, 55, 80, 81 and 107; 2, 13, 55, 80, 81 and 108; 2, 13, 55, 80, 81 and 109; 2, 13, 55, 80, 97 and 107; 2, 13, 55, 80, 97 and 108; 2, 13, 55, 80, 97 and 109; 2, 13, 55, 80, 98 and 107; 2, 13, 55, 80, 98 and 108; 2, 13, 55, 80, 98 and 109; 2, 13, 55, 81, 97 and 107; 2, 13, 55, 81, 97 and 108; 2, 13, 55, 81, 97 and 109; 2, 13, 55, 81, 98 and 107; 2, 13, 55, 81, 98 and 108; 2, 13, 55, 81, 98 and 109; 2, 13, 55, 82, 81 and 107; 2, 13, 55, 82, 81 and 108; 2, 13, 55, 82, 81 and 109; 2, 13, 55, 82, 97 and 107; 2, 13, 55, 82, 97 and 108; 2, 13, 55, 82, 97 and 109; 2, 13, 55, 82, 98 and 107; 2, 13, 55, 82, 98 and 108; or 2, 13, 55, 82, 98 and 109.

Also preferably, the vaccine provides combinations of at least six discrete immunization peptides comprising SEQ ID NO:14, more preferably provides immunization polypeptides consisting of amino acids of SEQ ID NOs:1, 14, 53, 80, 81 and 107; 1, 14, 53, 80, 81 and 108; 1, 14, 53, 80, 81 and 109; 1, 14, 53, 80, 97 and 107; 1, 14, 53, 80, 97 and 108; 1, 14, 53, 80, 97 and 109; 1, 14, 53, 80, 98 and 107; 1, 14, 53, 80, 98 and 108; 1, 14, 53, 80, 98 and 109; 1, 14, 53, 81, 97 and 107; 1, 14, 53, 81, 97 and 108; 1, 14, 53, 81, 97 and 109; 1, 14, 53, 81, 98 and 107; 1, 14, 53, 81, 98 and 108; 1, 14, 53, 81, 98 and 109; 1, 14, 53, 82, 81 and 107; 1, 14, 53, 82, 81 and 108; 1, 14, 53, 82, 81 and 109; 1, 14, 53, 82, 97 and 107; 1, 14, 53, 82, 97 and 108; 1, 14, 53, 82, 97 and 109; 1, 14, 53, 82, 98 and 107; 1, 14, 53, 82, 98 and 108; 1, 14, 53, 82, 98 and 109; 1, 14, 54, 80, 81 and 107; 1, 14, 54, 80, 81 and 108; 1, 14, 54, 80, 81 and 109; 1, 14, 54, 80, 97 and 107; 1, 14, 54, 80, 97 and 108; 1, 14, 54, 80, 97 and 109; 1, 14, 54, 80, 98 and 107; 1, 14, 54, 80, 98 and 108; 1, 14, 54, 80, 98 and 109; 1, 14, 54, 81, 97 and 107; 1, 14, 54, 81, 97 and 108; 1, 14, 54, 81, 97 and 109; 1, 14, 54, 81, 98 and 107; 1, 14, 54, 81, 98 and 108; 1, 14, 54, 81, 98 and 109; 1, 14, 54, 82, 81 and 107; 1, 14, 54, 82, 81 and 108; 1, 14, 54, 82, 81 and 109; 1, 14, 54, 82, 97 and 107; 1, 14, 54, 82, 97 and 108; 1, 14, 54, 82, 97 and 109; 1, 14, 54, 82, 98 and 107; 1, 14, 54, 82, 98 and 108; 1, 14, 54, 82, 98 and 109; 1, 14, 55, 80, 81 and 107; 1, 14, 55, 80, 81 and 108; 1, 14, 55, 80, 81 and 109; 1, 14, 55, 80, 97 and 107; 1, 14, 55, 80, 97 and 108; 1, 14, 55, 80, 97 and 109; 1, 14, 55, 80, 98 and 107; 1, 14, 55, 80, 98 and 108; 1, 14, 55, 80, 98 and 109; 1, 14, 55, 81, 97 and 107; 1, 14, 55, 81, 97 and 108; 1, 14, 55, 81, 97 and 109; 1, 14, 55, 81, 98 and 107; 1, 14, 55, 81, 98 and 108; 1, 14, 55, 81, 98 and 109; 1, 14, 55, 82, 81 and 107; 1, 14, 55, 82, 81 and 108; 1, 14, 55, 82, 81 and 109; 1, 14, 55, 82, 97 and 107; 1, 14, 55, 82, 97 and 108; 1, 14, 55, 82, 97 and 109; 1, 14, 55, 82, 98 and 107; 1, 14, 55, 82, 98 and 108; 1, 14, 55, 82, 98 and 109; 2, 14, 53, 80, 81 and 107; 2, 14, 53, 80, 81 and 108; 2, 14, 53, 80, 81 and 109; 2, 14, 53, 80, 97 and 107; 2, 14, 53, 80, 97 and 108; 2, 14, 53, 80, 97 and 109; 2, 14, 53, 80, 98 and 107; 2, 14, 53, 80, 98 and 108; 2, 14, 53, 80, 98 and 109; 2, 14, 53, 81, 97 and 107; 2, 14, 53, 81, 97 and 108; 2, 14, 53, 81, 97 and 109; 2, 14, 53, 81, 98 and 107; 2, 14, 53, 81, 98 and 108; 2, 14, 53, 81, 98 and 109; 2, 14, 53, 82, 81 and 107; 2, 14, 53, 82, 81 and 108; 2, 14, 53, 82, 81 and 109; 2, 14, 53, 82, 97 and 107; 2, 14, 53, 82, 97 and 108; 2, 14, 53, 82, 97 and 109; 2, 14, 53, 82, 98 and 107; 2, 14, 53, 82, 98 and 108; 2, 14, 53, 82, 98 and 109; 2, 14, 54, 80, 81 and 107; 2, 14, 54, 80, 81 and 108; 2, 14, 54, 80, 81 and 109; 2, 14, 54, 80, 97 and 107; 2, 14, 54, 80, 97 and 108; 2, 14, 54, 80, 97 and 109; 2, 14, 54, 80, 98 and 107; 2, 14, 54, 80, 98 and 108; 2, 14, 54, 80, 98 and 109; 2, 14, 54, 81, 97 and 107; 2, 14, 54, 81, 97 and 108; 2, 14, 54, 81, 97 and 109; 2, 14, 54, 81, 98 and 107; 2, 14, 54, 81, 98 and 108; 2, 14, 54, 81, 98 and 109; 2, 14, 54, 82, 81 and 107; 2, 14, 54, 82, 81 and 108; 2, 14, 54, 82, 81 and 109; 2, 14, 54, 82, 97 and 107; 2, 14, 54, 82, 97 and 108; 2, 14, 54, 82, 97 and 109; 2, 14, 54, 82, 98 and 107; 2, 14, 54, 82, 98 and 108; 2, 14, 54, 82, 98 and 109; 2, 14, 55, 80, 81 and 107; 2, 14, 55, 80, 81 and 108; 2, 14, 55, 80, 81 and 109; 2, 14, 55, 80, 97 and 107; 2, 14, 55, 80, 97 and 108; 2, 14, 55, 80, 97 and 109; 2, 14, 55, 80, 98 and 107; 2, 14, 55, 80, 98 and 108; 2, 14, 55, 80, 98 and 109; 2, 14, 55, 81, 97 and 107; 2, 14, 55, 81, 97 and 108; 2, 14, 55, 81, 97 and 109; 2, 14, 55, 81, 98 and 107; 2, 14, 55, 81, 98 and 108; 2, 14, 55, 81, 98 and 109; 2, 14, 55, 82, 81 and 107; 2, 14, 55, 82, 81 and 108; 2, 14, 55, 82, 81 and 109; 2, 14, 55, 82, 97 and 107; 2, 14, 55, 82, 97 and 108; 2, 14, 55, 82, 97 and 109; 2, 14, 55, 82, 98 and 107; 2, 14, 55, 82, 98 and 108; or 2, 14, 55, 82, 98 and 109.

Also preferably, the vaccine provides combinations of at least six discrete immunization peptides comprising SEQ ID NO:17, more preferably provides immunization polypeptides consisting of amino acids of SEQ ID NOs:1, 17, 53, 80, 81 and 107; 1, 17, 53, 80, 81 and 108; 1, 17, 53, 80, 81 and 109; 1, 17, 53, 80, 97 and 107; 1, 17, 53, 80, 97 and 108; 1, 17, 53, 80, 97 and 109; 1, 17, 53, 80, 98 and 107; 1, 17, 53, 80, 98 and 108; 1, 17, 53, 80, 98 and 109; 1, 17, 53, 81, 97 and 107; 1, 17, 53, 81, 97 and 108; 1, 17, 53, 81, 97 and 109; 1, 17, 53, 81, 98 and 107; 1, 17, 53, 81, 98 and 108; 1, 17, 53, 81, 98 and 109; 1, 17, 53, 82, 81 and 107; 1, 17, 53, 82, 81 and 108; 1, 17, 53, 82, 81 and 109; 1, 17, 53, 82, 97 and 107; 1, 17, 53, 82, 97 and 108; 1, 17, 53, 82, 97 and 109; 1, 17, 53, 82, 98 and 107; 1, 17, 53, 82, 98 and 108; 1, 17, 53, 82, 98 and 109; 1, 17, 54, 80, 81 and 107; 1, 17, 54, 80, 81 and 108; 1, 17, 54, 80, 81 and 109; 1, 17, 54, 80, 97 and 107; 1, 17, 54, 80, 97 and 108; 1, 17, 54, 80, 97 and 109; 1, 17, 54, 80, 98 and 107; 1, 17, 54, 80, 98 and 108; 1, 17, 54, 80, 98 and 109; 1, 17, 54, 81, 97 and 107; 1, 17, 54, 81, 97 and 108; 1, 17, 54, 81, 97 and 109; 1, 17, 54, 81, 98 and 107; 1, 17, 54, 81, 98 and 108; 1, 17, 54, 81, 98 and 109; 1, 17, 54, 82, 81 and 107; 1, 17, 54, 82, 81 and 108; 1, 17, 54, 82, 81 and 109; 1, 17, 54, 82, 97 and 107; 1, 17, 54, 82, 97 and 108; 1, 17, 54, 82, 97 and 109; 1, 17, 54, 82, 98 and 107; 1, 17, 54, 82, 98 and 108; 1, 17, 54, 82, 98 and 109; 1, 17, 55, 80, 81 and 107; 1, 17, 55, 80, 81 and 108; 1, 17, 55, 80, 81 and 109; 1, 17, 55, 80, 97 and 107; 1, 17, 55, 80, 97 and 108; 1, 17, 55, 80, 97 and 109; 1, 17, 55, 80, 98 and 107; 1, 17, 55, 80, 98 and 108; 1, 17, 55, 80, 98 and 109; 1, 17, 55, 81, 97 and 107; 1, 17, 55, 81, 97 and 108; 1, 17, 55, 81, 97 and 109; 1, 17, 55, 81, 98 and 107; 1, 17, 55, 81, 98 and 108; 1, 17, 55, 81, 98 and 109; 1, 17, 55, 82, 81 and 107; 1, 17, 55, 82, 81 and 108; 1, 17, 55, 82, 81 and 109; 1, 17, 55, 82, 97 and 107; 1, 17, 55, 82, 97 and 108; 1, 17, 55, 82, 97 and 109; 1, 17, 55, 82, 98 and 107; 1, 17, 55, 82, 98 and 108; 1, 17, 55, 82, 98 and 109; 2, 17, 53, 80, 81 and 107; 2, 17, 53, 80, 81 and 108; 2, 17, 53, 80, 81 and 109; 2, 17, 53, 80, 97 and 107; 2, 17, 53, 80, 97 and 108; 2, 17, 53, 80, 97 and 109; 2, 17, 53, 80, 98 and 107; 2, 17, 53, 80, 98 and 108; 2, 17, 53, 80, 98 and 109; 2, 17, 53, 81, 97 and 107; 2, 17, 53, 81, 97 and 108; 2, 17, 53, 81, 97 and 109; 2, 17, 53, 81, 98 and 107; 2, 17, 53, 81, 98 and 108; 2, 17, 53, 81, 98 and 109; 2, 17, 53, 82, 81 and 107; 2, 17, 53, 82, 81 and 108; 2, 17, 53, 82, 81 and 109; 2, 17, 53, 82, 97 and 107; 2, 17, 53, 82, 97 and 108; 2, 17, 53, 82, 97 and 109; 2, 17, 53, 82, 98 and 107; 2, 17, 53, 82, 98 and 108; 2, 17, 53, 82, 98 and 109; 2, 17, 54, 80, 81 and 107; 2, 17, 54, 80, 81 and 108; 2, 17, 54, 80, 81 and 109; 2, 17, 54, 80, 97 and 107; 2, 17, 54, 80, 97 and 108; 2, 17, 54, 80, 97 and 109; 2, 17, 54, 80, 98 and 107; 2, 17, 54, 80, 98 and 108; 2, 17, 54, 80, 98 and 109; 2, 17, 54, 81, 97 and 107; 2, 17, 54, 81, 97 and 108; 2, 17, 54, 81, 97 and 109; 2, 17, 54, 81, 98 and 107; 2, 17, 54, 81, 98 and 108; 2, 17, 54, 81, 98 and 109; 2, 17, 54, 82, 81 and 107; 2, 17, 54, 82, 81 and 108; 2, 17, 54, 82, 81 and 109; 2, 17, 54, 82, 97 and 107; 2, 17, 54, 82, 97 and 108; 2, 17, 54, 82, 97 and 109; 2, 17, 54, 82, 98 and 107; 2, 17, 54, 82, 98 and 108; 2, 17, 54, 82, 98 and 109; 2, 17, 55, 80, 81 and 107; 2, 17, 55, 80, 81 and 108; 2, 17, 55, 80, 81 and 109; 2, 17, 55, 80, 97 and 107; 2, 17, 55, 80, 97 and 108; 2, 17, 55, 80, 97 and 109; 2, 17, 55, 80, 98 and 107; 2, 17, 55, 80, 98 and 108; 2, 17, 55, 80, 98 and 109; 2, 17, 55, 81, 97 and 107; 2, 17, 55, 81, 97 and 108; 2, 17, 55, 81, 97 and 109; 2, 17, 55, 81, 98 and 107; 2, 17, 55, 81, 98 and 108; 2, 17, 55, 81, 98 and 109; 2, 17, 55, 82, 81 and 107; 2, 17, 55, 82, 81 and 108; 2, 17, 55, 82, 81 and 109; 2, 17, 55, 82, 97 and 107; 2, 17, 55, 82, 97 and 108; 2, 17, 55, 82, 97 and 109; 2, 17, 55, 82, 98 and 107; 2, 17, 55, 82, 98 and 108; or 2, 17, 55, 82, 98 and 109.

Also preferably, the vaccine provides combinations of at least six discrete immunization peptides comprising SEQ ID NO:18, more preferably provides immunization polypeptides consisting of amino acids of SEQ ID NOs:1, 18, 53, 80, 81 and 107; 1, 18, 53, 80, 81 and 108; 1, 18, 53, 80, 81 and 109; 1, 18, 53, 80, 97 and 107; 1, 18, 53, 80, 97 and 108; 1, 18, 53, 80, 97 and 109; 1, 18, 53, 80, 98 and 107; 1, 18, 53, 80, 98 and 108; 1, 18, 53, 80, 98 and 109; 1, 18, 53, 81, 97 and 107; 1, 18, 53, 81, 97 and 108; 1, 18, 53, 81, 97 and 109; 1, 18, 53, 81, 98 and 107; 1, 18, 53, 81, 98 and 108; 1, 18, 53, 81, 98 and 109; 1, 18, 53, 82, 81 and 107; 1, 18, 53, 82, 81 and 108; 1, 18, 53, 82, 81 and 109; 1, 18, 53, 82, 97 and 107; 1, 18, 53, 82, 97 and 108; 1, 18, 53, 82, 97 and 109; 1, 18, 53, 82, 98 and 107; 1, 18, 53, 82, 98 and 108; 1, 18, 53, 82, 98 and 109; 1, 18, 54, 80, 81 and 107; 1, 18, 54, 80, 81 and 108; 1, 18, 54, 80, 81 and 109; 1, 18, 54, 80, 97 and 107; 1, 18, 54, 80, 97 and 108; 1, 18, 54, 80, 97 and 109; 1, 18, 54, 80, 98 and 107; 1, 18, 54, 80, 98 and 108; 1, 18, 54, 80, 98 and 109; 1, 18, 54, 81, 97 and 107; 1, 18, 54, 81, 97 and 108; 1, 18, 54, 81, 97 and 109; 1, 18, 54, 81, 98 and 107; 1, 18, 54, 81, 98 and 108; 1, 18, 54, 81, 98 and 109; 1, 18, 54, 82, 81 and 107; 1, 18, 54, 82, 81 and 108; 1, 18, 54, 82, 81 and 109; 1, 18, 54, 82, 97 and 107; 1, 18, 54, 82, 97 and 108; 1, 18, 54, 82, 97 and 109; 1, 18, 54, 82, 98 and 107; 1, 18, 54, 82, 98 and 108; 1, 18, 54, 82, 98 and 109; 1, 18, 55, 80, 81 and 107; 1, 18, 55, 80, 81 and 108; 1, 18, 55, 80, 81 and 109; 1, 18, 55, 80, 97 and 107; 1, 18, 55, 80, 97 and 108; 1, 18, 55, 80, 97 and 109; 1, 18, 55, 80, 98 and 107; 1, 18, 55, 80, 98 and 108; 1, 18, 55, 80, 98 and 109; 1, 18, 55, 81, 97 and 107; 1, 18, 55, 81, 97 and 108; 1, 18, 55, 81, 97 and 109; 1, 18, 55, 81, 98 and 107; 1, 18, 55, 81, 98 and 108; 1, 18, 55, 81, 98 and 109; 1, 18, 55, 82, 81 and 107; 1, 18, 55, 82, 81 and 108; 1, 18, 55, 82, 81 and 109; 1, 18, 55, 82, 97 and 107; 1, 18, 55, 82, 97 and 108; 1, 18, 55, 82, 97 and 109; 1, 18, 55, 82, 98 and 107; 1, 18, 55, 82, 98 and 108; 1, 18, 55, 82, 98 and 109; 2, 18, 53, 80, 81 and 107; 2, 18, 53, 80, 81 and 108; 2, 18, 53, 80, 81 and 109; 2, 18, 53, 80, 97 and 107; 2, 18, 53, 80, 97 and 108; 2, 18, 53, 80, 97 and 109; 2, 18, 53, 80, 98 and 107; 2, 18, 53, 80, 98 and 108; 2, 18, 53, 80, 98 and 109; 2, 18, 53, 81, 97 and 107; 2, 18, 53, 81, 97 and 108; 2, 18, 53, 81, 97 and 109; 2, 18, 53, 81, 98 and 107; 2, 18, 53, 81, 98 and 108; 2, 18, 53, 81, 98 and 109; 2, 18, 53, 82, 81 and 107; 2, 18, 53, 82, 81 and 108; 2, 18, 53, 82, 81 and 109; 2, 18, 53, 82, 97 and 107; 2, 18, 53, 82, 97 and 108; 2, 18, 53, 82, 97 and 109; 2, 18, 53, 82, 98 and 107; 2, 18, 53, 82, 98 and 108; 2, 18, 53, 82, 98 and 109; 2, 18, 54, 80, 81 and 107; 2, 18, 54, 80, 81 and 108; 2, 18, 54, 80, 81 and 109; 2, 18, 54, 80, 97 and 107; 2, 18, 54, 80, 97 and 108; 2, 18, 54, 80, 97 and 109; 2, 18, 54, 80, 98 and 107; 2, 18, 54, 80, 98 and 108; 2, 18, 54, 80, 98 and 109; 2, 18, 54, 81, 97 and 107; 2, 18, 54, 81, 97 and 108; 2, 18, 54, 81, 97 and 109; 2, 18, 54, 81, 98 and 107; 2, 18, 54, 81, 98 and 108; 2, 18, 54, 81, 98 and 109; 2, 18, 54, 82, 81 and 107; 2, 18, 54, 82, 81 and 108; 2, 18, 54, 82, 81 and 109; 2, 18, 54, 82, 97 and 107; 2, 18, 54, 82, 97 and 108; 2, 18, 54, 82, 97 and 109; 2, 18, 54, 82, 98 and 107; 2, 18, 54, 82, 98 and 108; 2, 18, 54, 82, 98 and 109; 2, 18, 55, 80, 81 and 107; 2, 18, 55, 80, 81 and 108; 2, 18, 55, 80, 81 and 109; 2, 18, 55, 80, 97 and 107; 2, 18, 55, 80, 97 and 108; 2, 18, 55, 80, 97 and 109; 2, 18, 55, 80, 98 and 107; 2, 18, 55, 80, 98 and 108; 2, 18, 55, 80, 98 and 109; 2, 18, 55, 81, 97 and 107; 2, 18, 55, 81, 97 and 108; 2, 18, 55, 81, 97 and 109; 2, 18, 55, 81, 98 and 107; 2, 18, 55, 81, 98 and 108; 2, 18, 55, 81, 98 and 109; 2, 18, 55, 82, 81 and 107; 2, 18, 55, 82, 81 and 108; 2, 18, 55, 82, 81 and 109; 2, 18, 55, 82, 97 and 107; 2, 18, 55, 82, 97 and 108; 2, 18, 55, 82, 97 and 109; 2, 18, 55, 82, 98 and 107; 2, 18, 55, 82, 98 and 108; or 2, 18, 55, 82, 98 and 109.

Also preferably, the vaccine provides combinations of at least six discrete immunization peptides comprising SEQ ID NO:53, more preferably provides immunization polypeptides consisting of amino acids of SEQ ID NOs:1, 11, 53, 80, 81 and 107; 1, 11, 53, 80, 81 and 108; 1, 11, 53, 80, 81 and 109; 1, 11, 53, 80, 97 and 107; 1, 11, 53, 80, 97 and 108; 1, 11, 53, 80, 97 and 109; 1, 11, 53, 80, 98 and 107; 1, 11, 53, 80, 98 and 108; 1, 11, 53, 80, 98 and 109; 1, 11, 53, 81, 97 and 107; 1, 11, 53, 81, 97 and 108; 1, 11, 53, 81, 97 and 109; 1, 11, 53, 81, 98 and 107; 1, 11, 53, 81, 98 and 108; 1, 11, 53, 81, 98 and 109; 1, 11, 53, 82, 81 and 107; 1, 11, 53, 82, 81 and 108; 1, 11, 53, 82, 81 and 109; 1, 11, 53, 82, 97 and 107; 1, 11, 53, 82, 97 and 108; 1, 11, 53, 82, 97 and 109; 1, 11, 53, 82, 98 and 107; 1, 11, 53, 82, 98 and 108; 1, 11, 53, 82, 98 and 109; 2, 11, 53, 80, 81 and 107; 2, 11, 53, 80, 81 and 108; 2, 11, 53, 80, 81 and 109; 2, 11, 53, 80, 97 and 107; 2, 11, 53, 80, 97 and 108; 2, 11, 53, 80, 97 and 109; 2, 11, 53, 80, 98 and 107; 2, 11, 53, 80, 98 and 108; 2, 11, 53, 80, 98 and 109; 2, 11, 53, 81, 97 and 107; 2, 11, 53, 81, 97 and 108; 2, 11, 53, 81, 97 and 109; 2, 11, 53, 81, 98 and 107; 2, 11, 53, 81, 98 and 108; 2, 11, 53, 81, 98 and 109; 2, 11, 53, 82, 81 and 107; 2, 11, 53, 82, 81 and 108; 2, 11, 53, 82, 81 and 109; 2, 11, 53, 82, 97 and 107; 2, 11, 53, 82, 97 and 108; 2, 11, 53, 82, 97 and 109; 2, 11, 53, 82, 98 and 107; 2, 11, 53, 82, 98 and 108; 2, 11, 53, 82, 98 and 109; 1, 13, 53, 80, 81 and 107; 1, 13, 53, 80, 81 and 108; 1, 13, 53, 80, 81 and 109; 1, 13, 53, 80, 97 and 107; 1, 13, 53, 80, 97 and 108; 1, 13, 53, 80, 97 and 109; 1, 13, 53, 80, 98 and 107; 1, 13, 53, 80, 98 and 108; 1, 13, 53, 80, 98 and 109; 1, 13, 53, 81, 97 and 107; 1, 13, 53, 81, 97 and 108; 1, 13, 53, 81, 97 and 109; 1, 13, 53, 81, 98 and 107; 1, 13, 53, 81, 98 and 108; 1, 13, 53, 81, 98 and 109; 1, 13, 53, 82, 81 and 107; 1, 13, 53, 82, 81 and 108; 1, 13, 53, 82, 81 and 109; 1, 13, 53, 82, 97 and 107; 1, 13, 53, 82, 97 and 108; 1, 13, 53, 82, 97 and 109; 1, 13, 53, 82, 98 and 107; 1, 13, 53, 82, 98 and 108; 1, 13, 53, 82, 98 and 109; 2, 13, 53, 80, 81 and 107; 2, 13, 53, 80, 81 and 108; 2, 13, 53, 80, 81 and 109; 2, 13, 53, 80, 97 and 107; 2, 13, 53, 80, 97 and 108; 2, 13, 53, 80, 97 and 109; 2, 13, 53, 80, 98 and 107; 2, 13, 53, 80, 98 and 108; 2, 13, 53, 80, 98 and 109; 2, 13, 53, 81, 97 and 107; 2, 13, 53, 81, 97 and 108; 2, 13, 53, 81, 97 and 109; 2, 13, 53, 81, 98 and 107; 2, 13, 53, 81, 98 and 108; 2, 13, 53, 81, 98 and 109; 2, 13, 53, 82, 81 and 107; 2, 13, 53, 82, 81 and 108; 2, 13, 53, 82, 81 and 109; 2, 13, 53, 82, 97 and 107; 2, 13, 53, 82, 97 and 108; 2, 13, 53, 82, 97 and 109; 2, 13, 53, 82, 98 and 107; 2, 13, 53, 82, 98 and 108; 2, 13, 53, 82, 98 and 109; 1, 14, 53, 80, 81 and 107; 1, 14, 53, 80, 81 and 108; 1, 14, 53, 80, 81 and 109; 1, 14, 53, 80, 97 and 107; 1, 14, 53, 80, 97 and 108; 1, 14, 53, 80, 97 and 109; 1, 14, 53, 80, 98 and 107; 1, 14, 53, 80, 98 and 108; 1, 14, 53, 80, 98 and 109; 1, 14, 53, 81, 97 and 107; 1, 14, 53, 81, 97 and 108; 1, 14, 53, 81, 97 and 109; 1, 14, 53, 81, 98 and 107; 1, 14, 53, 81, 98 and 108; 1, 14, 53, 81, 98 and 109; 1, 14, 53, 82, 81 and 107; 1, 14, 53, 82, 81 and 108; 1, 14, 53, 82, 81 and 109; 1, 14, 53, 82, 97 and 107; 1, 14, 53, 82, 97 and 108; 1, 14, 53, 82, 97 and 109; 1, 14, 53, 82, 98 and 107; 1, 14, 53, 82, 98 and 108; 1, 14, 53, 82, 98 and 109; 2, 14, 53, 80, 81 and 107; 2, 14, 53, 80, 81 and 108; 2, 14, 53, 80, 81 and 109; 2, 14, 53, 80, 97 and 107; 2, 14, 53, 80, 97 and 108; 2, 14, 53, 80, 97 and 109; 2, 14, 53, 80, 98 and 107; 2, 14, 53, 80, 98 and 108; 2, 14, 53, 80, 98 and 109; 2, 14, 53, 81, 97 and 107; 2, 14, 53, 81, 97 and 108; 2, 14, 53, 81, 97 and 109; 2, 14, 53, 81, 98 and 107; 2, 14, 53, 81, 98 and 108; 2, 14, 53, 81, 98 and 109; 2, 14, 53, 82, 81 and 107; 2, 14, 53, 82, 81 and 108; 2, 14, 53, 82, 81 and 109; 2, 14, 53, 82, 97 and 107; 2, 14, 53, 82, 97 and 108; 2, 14, 53, 82, 97 and 109; 2, 14, 53, 82, 98 and 107; 2, 14, 53, 82, 98 and 108; 2, 14, 53, 82, 98 and 109; 1, 17, 53, 80, 81 and 107; 1, 17, 53, 80, 81 and 108; 1, 17, 53, 80, 81 and 109; 1, 17, 53, 80, 97 and 107; 1, 17, 53, 80, 97 and 108; 1, 17, 53, 80, 97 and 109; 1, 17, 53, 80, 98 and 107; 1, 17, 53, 80, 98 and 108; 1, 17, 53, 80, 98 and 109; 1, 17, 53, 81, 97 and 107; 1, 17, 53, 81, 97 and 108; 1, 17, 53, 81, 97 and 109; 1, 17, 53, 81, 98 and 107; 1, 17, 53, 81, 98 and 108; 1, 17, 53, 81, 98 and 109; 1, 17, 53, 82, 81 and 107; 1, 17, 53, 82, 81 and 108; 1, 17, 53, 82, 81 and 109; 1, 17, 53, 82, 97 and 107; 1, 17, 53, 82, 97 and 108; 1, 17, 53, 82, 97 and 109; 1, 17, 53, 82, 98 and 107; 1, 17, 53, 82, 98 and 108; 1, 17, 53, 82, 98 and 109; 2, 17, 53, 80, 81 and 107; 2, 17, 53, 80, 81 and 108; 2, 17, 53, 80, 81 and 109; 2, 17, 53, 80, 97 and 107; 2, 17, 53, 80, 97 and 108; 2, 17, 53, 80, 97 and 109; 2, 17, 53, 80, 98 and 107; 2, 17, 53, 80, 98 and 108; 2, 17, 53, 80, 98 and 109; 2, 17, 53, 81, 97 and 107; 2, 17, 53, 81, 97 and 108; 2, 17, 53, 81, 97 and 109; 2, 17, 53, 81, 98 and 107; 2, 17, 53, 81, 98 and 108; 2, 17, 53, 81, 98 and 109; 2, 17, 53, 82, 81 and 107; 2, 17, 53, 82, 81 and 108; 2, 17, 53, 82, 81 and 109; 2, 17, 53, 82, 97 and 107; 2, 17, 53, 82, 97 and 108; 2, 17, 53, 82, 97 and 109; 2, 17, 53, 82, 98 and 107; 2, 17, 53, 82, 98 and 108; 2, 17, 53, 82, 98 and 109; 1, 18, 53, 80, 81 and 107; 1, 18, 53, 80, 81 and 108; 1, 18, 53, 80, 81 and 109; 1, 18, 53, 80, 97 and 107; 1, 18, 53, 80, 97 and 108; 1, 18, 53, 80, 97 and 109; 1, 18, 53, 80, 98 and 107; 1, 18, 53, 80, 98 and 108; 1, 18, 53, 80, 98 and 109; 1, 18, 53, 81, 97 and 107; 1, 18, 53, 81, 97 and 108; 1, 18, 53, 81, 97 and 109; 1, 18, 53, 81, 98 and 107; 1, 18, 53, 81, 98 and 108; 1, 18, 53, 81, 98 and 109; 1, 18, 53, 82, 81 and 107; 1, 18, 53, 82, 81 and 108; 1, 18, 53, 82, 81 and 109; 1, 18, 53, 82, 97 and 107; 1, 18, 53, 82, 97 and 108; 1, 18, 53, 82, 97 and 109; 1, 18, 53, 82, 98 and 107; 1, 18, 53, 82, 98 and 108; 1, 18, 53, 82, 98 and 109; 2, 18, 53, 80, 81 and 107; 2, 18, 53, 80, 81 and 108; 2, 18, 53, 80, 81 and 109; 2, 18, 53, 80, 97 and 107; 2, 18, 53, 80, 97 and 108; 2, 18, 53, 80, 97 and 109; 2, 18, 53, 80, 98 and 107; 2, 18, 53, 80, 98 and 108; 2, 18, 53, 80, 98 and 109; 2, 18, 53, 81, 97 and 107; 2, 18, 53, 81, 97 and 108; 2, 18, 53, 81, 97 and 109; 2, 18, 53, 81, 98 and 107; 2, 18, 53, 81, 98 and 108; 2, 18, 53, 81, 98 and 109; 2, 18, 53, 82, 81 and 107; 2, 18, 53, 82, 81 and 108; 2, 18, 53, 82, 81 and 109; 2, 18, 53, 82, 97 and 107; 2, 18, 53, 82, 97 and 108; 2, 18, 53, 82, 97 and 109; 2, 18, 53, 82, 98 and 107; 2, 18, 53, 82, 98 and 108; or 2, 18, 53, 82, 98 and 109.

Also preferably, the vaccine provides combinations of at least six discrete immunization peptides comprising SEQ ID NO:54, more preferably provides immunization polypeptides consisting of amino acids of SEQ ID NOs:1, 11, 54, 80, 81 and 107; 1, 11, 54, 80, 81 and 108; 1, 11, 54, 80, 81 and 109; 1, 11, 54, 80, 97 and 107; 1, 11, 54, 80, 97 and 108; 1, 11, 54, 80, 97 and 109; 1, 11, 54, 80, 98 and 107; 1, 11, 54, 80, 98 and 108; 1, 11, 54, 80, 98 and 109; 1, 11, 54, 81, 97 and 107; 1, 11, 54, 81, 97 and 108; 1, 11, 54, 81, 97 and 109; 1, 11, 54, 81, 98 and 107; 1, 11, 54, 81, 98 and 108; 1, 11, 54, 81, 98 and 109; 1, 11, 54, 82, 81 and 107; 1, 11, 54, 82, 81 and 108; 1, 11, 54, 82, 81 and 109; 1, 11, 54, 82, 97 and 107; 1, 11, 54, 82, 97 and 108; 1, 11, 54, 82, 97 and 109; 1, 11, 54, 82, 98 and 107; 1, 11, 54, 82, 98 and 108; 1, 11, 54, 82, 98 and 109; 2, 11, 54, 80, 81 and 107; 2, 11, 54, 80, 81 and 108; 2, 11, 54, 80, 81 and 109; 2, 11, 54, 80, 97 and 107; 2, 11, 54, 80, 97 and 108; 2, 11, 54, 80, 97 and 109; 2, 11, 54, 80, 98 and 107; 2, 11, 54, 80, 98 and 108; 2, 11, 54, 80, 98 and 109; 2, 11, 54, 81, 97 and 107; 2, 11, 54, 81, 97 and 108; 2, 11, 54, 81, 97 and 109; 2, 11, 54, 81, 98 and 107; 2, 11, 54, 81, 98 and 108; 2, 11, 54, 81, 98 and 109; 2, 11, 54, 82, 81 and 107; 2, 11, 54, 82, 81 and 108; 2, 11, 54, 82, 81 and 109; 2, 11, 54, 82, 97 and 107; 2, 11, 54, 82, 97 and 108; 2, 11, 54, 82, 97 and 109; 2, 11, 54, 82, 98 and 107; 2, 11, 54, 82, 98 and 108; 2, 11, 54, 82, 98 and 109; 1, 13, 54, 80, 81 and 107; 1, 13, 54, 80, 81 and 108; 1, 13, 54, 80, 81 and 109; 1, 13, 54, 80, 97 and 107; 1, 13, 54, 80, 97 and 108; 1, 13, 54, 80, 97 and 109; 1, 13, 54, 80, 98 and 107; 1, 13, 54, 80, 98 and 108; 1, 13, 54, 80, 98 and 109; 1, 13, 54, 81, 97 and 107; 1, 13, 54, 81, 97 and 108; 1, 13, 54, 81, 97 and 109; 1, 13, 54, 81, 98 and 107; 1, 13, 54, 81, 98 and 108; 1, 13, 54, 81, 98 and 109; 1, 13, 54, 82, 81 and 107; 1, 13, 54, 82, 81 and 108; 1, 13, 54, 82, 81 and 109; 1, 13, 54, 82, 97 and 107; 1, 13, 54, 82, 97 and 108; 1, 13, 54, 82, 97 and 109; 1, 13, 54, 82, 98 and 107; 1, 13, 54, 82, 98 and 108; 1, 13, 54, 82, 98 and 109; 2, 13, 54, 80, 81 and 107; 2, 13, 54, 80, 81 and 108; 2, 13, 54, 80, 81 and 109; 2, 13, 54, 80, 97 and 107; 2, 13, 54, 80, 97 and 108; 2, 13, 54, 80, 97 and 109; 2, 13, 54, 80, 98 and 107; 2, 13, 54, 80, 98 and 108; 2, 13, 54, 80, 98 and 109; 2, 13, 54, 81, 97 and 107; 2, 13, 54, 81, 97 and 108; 2, 13, 54, 81, 97 and 109; 2, 13, 54, 81, 98 and 107; 2, 13, 54, 81, 98 and 108; 2, 13, 54, 81, 98 and 109; 2, 13, 54, 82, 81 and 107; 2, 13, 54, 82, 81 and 108; 2, 13, 54, 82, 81 and 109; 2, 13, 54, 82, 97 and 107; 2, 13, 54, 82, 97 and 108; 2, 13, 54, 82, 97 and 109; 2, 13, 54, 82, 98 and 107; 2, 13, 54, 82, 98 and 108; 2, 13, 54, 82, 98 and 109; 1, 14, 54, 80, 81 and 107; 1, 14, 54, 80, 81 and 108; 1, 14, 54, 80, 81 and 109; 1, 14, 54, 80, 97 and 107; 1, 14, 54, 80, 97 and 108; 1, 14, 54, 80, 97 and 109; 1, 14, 54, 80, 98 and 107; 1, 14, 54, 80, 98 and 108; 1, 14, 54, 80, 98 and 109; 1, 14, 54, 81, 97 and 107; 1, 14, 54, 81, 97 and 108; 1, 14, 54, 81, 97 and 109; 1, 14, 54, 81, 98 and 107; 1, 14, 54, 81, 98 and 108; 1, 14, 54, 81, 98 and 109; 1, 14, 54, 82, 81 and 107; 1, 14, 54, 82, 81 and 108; 1, 14, 54, 82, 81 and 109; 1, 14, 54, 82, 97 and 107; 1, 14, 54, 82, 97 and 108; 1, 14, 54, 82, 97 and 109; 1, 14, 54, 82, 98 and 107; 1, 14, 54, 82, 98 and 108; 1, 14, 54, 82, 98 and 109; 2, 14, 54, 80, 81 and 107; 2, 14, 54, 80, 81 and 108; 2, 14, 54, 80, 81 and 109; 2, 14, 54, 80, 97 and 107; 2, 14, 54, 80, 97 and 108; 2, 14, 54, 80, 97 and 109; 2, 14, 54, 80, 98 and 107; 2, 14, 54, 80, 98 and 108; 2, 14, 54, 80, 98 and 109; 2, 14, 54, 81, 97 and 107; 2, 14, 54, 81, 97 and 108; 2, 14, 54, 81, 97 and 109; 2, 14, 54, 81, 98 and 107; 2, 14, 54, 81, 98 and 108; 2, 14, 54, 81, 98 and 109; 2, 14, 54, 82, 81 and 107; 2, 14, 54, 82, 81 and 108; 2, 14, 54, 82, 81 and 109; 2, 14, 54, 82, 97 and 107; 2, 14, 54, 82, 97 and 108; 2, 14, 54, 82, 97 and 109; 2, 14, 54, 82, 98 and 107; 2, 14, 54, 82, 98 and 108; 2, 14, 54, 82, 98 and 109; 1, 17, 54, 80, 81 and 107; 1, 17, 54, 80, 81 and 108; 1, 17, 54, 80, 81 and 109; 1, 17, 54, 80, 97 and 107; 1, 17, 54, 80, 97 and 108; 1, 17, 54, 80, 97 and 109; 1, 17, 54, 80, 98 and 107; 1, 17, 54, 80, 98 and 108; 1, 17, 54, 80, 98 and 109; 1, 17, 54, 81, 97 and 107; 1, 17, 54, 81, 97 and 108; 1, 17, 54, 81, 97 and 109; 1, 17, 54, 81, 98 and 107; 1, 17, 54, 81, 98 and 108; 1, 17, 54, 81, 98 and 109; 1, 17, 54, 82, 81 and 107; 1, 17, 54, 82, 81 and 108; 1, 17, 54, 82, 81 and 109; 1, 17, 54, 82, 97 and 107; 1, 17, 54, 82, 97 and 108; 1, 17, 54, 82, 97 and 109; 1, 17, 54, 82, 98 and 107; 1, 17, 54, 82, 98 and 108; 1, 17, 54, 82, 98 and 109; 2, 17, 54, 80, 81 and 107; 2, 17, 54, 80, 81 and 108; 2, 17, 54, 80, 81 and 109; 2, 17, 54, 80, 97 and 107; 2, 17, 54, 80, 97 and 108; 2, 17, 54, 80, 97 and 109; 2, 17, 54, 80, 98 and 107; 2, 17, 54, 80, 98 and 108; 2, 17, 54, 80, 98 and 109; 2, 17, 54, 81, 97 and 107; 2, 17, 54, 81, 97 and 108; 2, 17, 54, 81, 97 and 109; 2, 17, 54, 81, 98 and 107; 2, 17, 54, 81, 98 and 108; 2, 17, 54, 81, 98 and 109; 2, 17, 54, 82, 81 and 107; 2, 17, 54, 82, 81 and 108; 2, 17, 54, 82, 81 and 109; 2, 17, 54, 82, 97 and 107; 2, 17, 54, 82, 97 and 108; 2, 17, 54, 82, 97 and 109; 2, 17, 54, 82, 98 and 107; 2, 17, 54, 82, 98 and 108; 2, 17, 54, 82, 98 and 109; 1, 18, 54, 80, 81 and 107; 1, 18, 54, 80, 81 and 108; 1, 18, 54, 80, 81 and 109; 1, 18, 54, 80, 97 and 107; 1, 18, 54, 80, 97 and 108; 1, 18, 54, 80, 97 and 109; 1, 18, 54, 80, 98 and 107; 1, 18, 54, 80, 98 and 108; 1, 18, 54, 80, 98 and 109; 1, 18, 54, 81, 97 and 107; 1, 18, 54, 81, 97 and 108; 1, 18, 54, 81, 97 and 109; 1, 18, 54, 81, 98 and 107; 1, 18, 54, 81, 98 and 108; 1, 18, 54, 81, 98 and 109; 1, 18, 54, 82, 81 and 107; 1, 18, 54, 82, 81 and 108; 1, 18, 54, 82, 81 and 109; 1, 18, 54, 82, 97 and 107; 1, 18, 54, 82, 97 and 108; 1, 18, 54, 82, 97 and 109; 1, 18, 54, 82, 98 and 107; 1, 18, 54, 82, 98 and 108; 1, 18, 54, 82, 98 and 109; 2, 18, 54, 80, 81 and 107; 2, 18, 54, 80, 81 and 108; 2, 18, 54, 80, 81 and 109; 2, 18, 54, 80, 97 and 107; 2, 18, 54, 80, 97 and 108; 2, 18, 54, 80, 97 and 109; 2, 18, 54, 80, 98 and 107; 2, 18, 54, 80, 98 and 108; 2, 18, 54, 80, 98 and 109; 2, 18, 54, 81, 97 and 107; 2, 18, 54, 81, 97 and 108; 2, 18, 54, 81, 97 and 109; 2, 18, 54, 81, 98 and 107; 2, 18, 54, 81, 98 and 108; 2, 18, 54, 81, 98 and 109; 2, 18, 54, 82, 81 and 107; 2, 18, 54, 82, 81 and 108; 2, 18, 54, 82, 81 and 109; 2, 18, 54, 82, 97 and 107; 2, 18, 54, 82, 97 and 108; 2, 18, 54, 82, 97 and 109; 2, 18, 54, 82, 98 and 107; 2, 18, 54, 82, 98 and 108; or 2, 18, 54, 82, 98 and 109.

Also preferably, the vaccine provides combinations of at least six discrete immunization peptides comprising SEQ ID NO:55, more preferably provides immunization polypeptides consisting of amino acids of SEQ ID NOs:1, 11, 55, 80, 81 and 107; 1, 11, 55, 80, 81 and 108; 1, 11, 55, 80, 81 and 109; 1, 11, 55, 80, 97 and 107; 1, 11, 55, 80, 97 and 108; 1, 11, 55, 80, 97 and 109; 1, 11, 55, 80, 98 and 107; 1, 11, 55, 80, 98 and 108; 1, 11, 55, 80, 98 and 109; 1, 11, 55, 81, 97 and 107; 1, 11, 55, 81, 97 and 108; 1, 11, 55, 81, 97 and 109; 1, 11, 55, 81, 98 and 107; 1, 11, 55, 81, 98 and 108; 1, 11, 55, 81, 98 and 109; 1, 11, 55, 82, 81 and 107; 1, 11, 55, 82, 81 and 108; 1, 11, 55, 82, 81 and 109; 1, 11, 55, 82, 97 and 107; 1, 11, 55, 82, 97 and 108; 1, 11, 55, 82, 97 and 109; 1, 11, 55, 82, 98 and 107; 1, 11, 55, 82, 98 and 108; 1, 11, 55, 82, 98 and 109; 2, 11, 55, 80, 81 and 107; 2, 11, 55, 80, 81 and 108; 2, 11, 55, 80, 81 and 109; 2, 11, 55, 80, 97 and 107; 2, 11, 55, 80, 97 and 108; 2, 11, 55, 80, 97 and 109; 2, 11, 55, 80, 98 and 107; 2, 11, 55, 80, 98 and 108; 2, 11, 55, 80, 98 and 109; 2, 11, 55, 81, 97 and 107; 2, 11, 55, 81, 97 and 108; 2, 11, 55, 81, 97 and 109; 2, 11, 55, 81, 98 and 107; 2, 11, 55, 81, 98 and 108; 2, 11, 55, 81, 98 and 109; 2, 11, 55, 82, 81 and 107; 2, 11, 55, 82, 81 and 108; 2, 11, 55, 82, 81 and 109; 2, 11, 55, 82, 97 and 107; 2, 11, 55, 82, 97 and 108; 2, 11, 55, 82, 97 and 109; 2, 11, 55, 82, 98 and 107; 2, 11, 55, 82, 98 and 108; 2, 11, 55, 82, 98 and 109; 1, 13, 55, 80, 81 and 107; 1, 13, 55, 80, 81 and 108; 1, 13, 55, 80, 81 and 109; 1, 13, 55, 80, 97 and 107; 1, 13, 55, 80, 97 and 108; 1, 13, 55, 80, 97 and 109; 1, 13, 55, 80, 98 and 107; 1, 13, 55, 80, 98 and 108; 1, 13, 55, 80, 98 and 109; 1, 13, 55, 81, 97 and 107; 1, 13, 55, 81, 97 and 108; 1, 13, 55, 81, 97 and 109; 1, 13, 55, 81, 98 and 107; 1, 13, 55, 81, 98 and 108; 1, 13, 55, 81, 98 and 109; 1, 13, 55, 82, 81 and 107; 1, 13, 55, 82, 81 and 108; 1, 13, 55, 82, 81 and 109; 1, 13, 55, 82, 97 and 107; 1, 13, 55, 82, 97 and 108; 1, 13, 55, 82, 97 and 109; 1, 13, 55, 82, 98 and 107; 1, 13, 55, 82, 98 and 108; 1, 13, 55, 82, 98 and 109; 2, 13, 55, 80, 81 and 107; 2, 13, 55, 80, 81 and 108; 2, 13, 55, 80, 81 and 109; 2, 13, 55, 80, 97 and 107; 2, 13, 55, 80, 97 and 108; 2, 13, 55, 80, 97 and 109; 2, 13, 55, 80, 98 and 107; 2, 13, 55, 80, 98 and 108; 2, 13, 55, 80, 98 and 109; 2, 13, 55, 81, 97 and 107; 2, 13, 55, 81, 97 and 108; 2, 13, 55, 81, 97 and 109; 2, 13, 55, 81, 98 and 107; 2, 13, 55, 81, 98 and 108; 2, 13, 55, 81, 98 and 109; 2, 13, 55, 82, 81 and 107; 2, 13, 55, 82, 81 and 108; 2, 13, 55, 82, 81 and 109; 2, 13, 55, 82, 97 and 107; 2, 13, 55, 82, 97 and 108; 2, 13, 55, 82, 97 and 109; 2, 13, 55, 82, 98 and 107; 2, 13, 55, 82, 98 and 108; 2, 13, 55, 82, 98 and 109; 1, 14, 55, 80, 81 and 107; 1, 14, 55, 80, 81 and 108; 1, 14, 55, 80, 81 and 109; 1, 14, 55, 80, 97 and 107; 1, 14, 55, 80, 97 and 108; 1, 14, 55, 80, 97 and 109; 1, 14, 55, 80, 98 and 107; 1, 14, 55, 80, 98 and 108; 1, 14, 55, 80, 98 and 109; 1, 14, 55, 81, 97 and 107; 1, 14, 55, 81, 97 and 108; 1, 14, 55, 81, 97 and 109; 1, 14, 55, 81, 98 and 107; 1, 14, 55, 81, 98 and 108; 1, 14, 55, 81, 98 and 109; 1, 14, 55, 82, 81 and 107; 1, 14, 55, 82, 81 and 108; 1, 14, 55, 82, 81 and 109; 1, 14, 55, 82, 97 and 107; 1, 14, 55, 82, 97 and 108; 1, 14, 55, 82, 97 and 109; 1, 14, 55, 82, 98 and 107; 1, 14, 55, 82, 98 and 108; 1, 14, 55, 82, 98 and 109; 2, 14, 55, 80, 81 and 107; 2, 14, 55, 80, 81 and 108; 2, 14, 55, 80, 81 and 109; 2, 14, 55, 80, 97 and 107; 2, 14, 55, 80, 97 and 108; 2, 14, 55, 80, 97 and 109; 2, 14, 55, 80, 98 and 107; 2, 14, 55, 80, 98 and 108; 2, 14, 55, 80, 98 and 109; 2, 14, 55, 81, 97 and 107; 2, 14, 55, 81, 97 and 108; 2, 14, 55, 81, 97 and 109; 2, 14, 55, 81, 98 and 107; 2, 14, 55, 81, 98 and 108; 2, 14, 55, 81, 98 and 109; 2, 14, 55, 82, 81 and 107; 2, 14, 55, 82, 81 and 108; 2, 14, 55, 82, 81 and 109; 2, 14, 55, 82, 97 and 107; 2, 14, 55, 82, 97 and 108; 2, 14, 55, 82, 97 and 109; 2, 14, 55, 82, 98 and 107; 2, 14, 55, 82, 98 and 108; 2, 14, 55, 82, 98 and 109; 1, 17, 55, 80, 81 and 107; 1, 17, 55, 80, 81 and 108; 1, 17, 55, 80, 81 and 109; 1, 17, 55, 80, 97 and 107; 1, 17, 55, 80, 97 and 108; 1, 17, 55, 80, 97 and 109; 1, 17, 55, 80, 98 and 107; 1, 17, 55, 80, 98 and 108; 1, 17, 55, 80, 98 and 109; 1, 17, 55, 81, 97 and 107; 1, 17, 55, 81, 97 and 108; 1, 17, 55, 81, 97 and 109; 1, 17, 55, 81, 98 and 107; 1, 17, 55, 81, 98 and 108; 1, 17, 55, 81, 98 and 109; 1, 17, 55, 82, 81 and 107; 1, 17, 55, 82, 81 and 108; 1, 17, 55, 82, 81 and 109; 1, 17, 55, 82, 97 and 107; 1, 17, 55, 82, 97 and 108; 1, 17, 55, 82, 97 and 109; 1, 17, 55, 82, 98 and 107; 1, 17, 55, 82, 98 and 108; 1, 17, 55, 82, 98 and 109; 2, 17, 55, 80, 81 and 107; 2, 17, 55, 80, 81 and 108; 2, 17, 55, 80, 81 and 109; 2, 17, 55, 80, 97 and 107; 2, 17, 55, 80, 97 and 108; 2, 17, 55, 80, 97 and 109; 2, 17, 55, 80, 98 and 107; 2, 17, 55, 80, 98 and 108; 2, 17, 55, 80, 98 and 109; 2, 17, 55, 81, 97 and 107; 2, 17, 55, 81, 97 and 108; 2, 17, 55, 81, 97 and 109; 2, 17, 55, 81, 98 and 107; 2, 17, 55, 81, 98 and 108; 2, 17, 55, 81, 98 and 109; 2, 17, 55, 82, 81 and 107; 2, 17, 55, 82, 81 and 108; 2, 17, 55, 82, 81 and 109; 2, 17, 55, 82, 97 and 107; 2, 17, 55, 82, 97 and 108; 2, 17, 55, 82, 97 and 109; 2, 17, 55, 82, 98 and 107; 2, 17, 55, 82, 98 and 108; 2, 17, 55, 82, 98 and 109; 1, 18, 55, 80, 81 and 107; 1, 18, 55, 80, 81 and 108; 1, 18, 55, 80, 81 and 109; 1, 18, 55, 80, 97 and 107; 1, 18, 55, 80, 97 and 108; 1, 18, 55, 80, 97 and 109; 1, 18, 55, 80, 98 and 107; 1, 18, 55, 80, 98 and 108; 1, 18, 55, 80, 98 and 109; 1, 18, 55, 81, 97 and 107; 1, 18, 55, 81, 97 and 108; 1, 18, 55, 81, 97 and 109; 1, 18, 55, 81, 98 and 107; 1, 18, 55, 81, 98 and 108; 1, 18, 55, 81, 98 and 109; 1, 18, 55, 82, 81 and 107; 1, 18, 55, 82, 81 and 108; 1, 18, 55, 82, 81 and 109; 1, 18, 55, 82, 97 and 107; 1, 18, 55, 82, 97 and 108; 1, 18, 55, 82, 97 and 109; 1, 18, 55, 82, 98 and 107; 1, 18, 55, 82, 98 and 108; 1, 18, 55, 82, 98 and 109; 2, 18, 55, 80, 81 and 107; 2, 18, 55, 80, 81 and 108; 2, 18, 55, 80, 81 and 109; 2, 18, 55, 80, 97 and 107; 2, 18, 55, 80, 97 and 108; 2, 18, 55, 80, 97 and 109; 2, 18, 55, 80, 98 and 107; 2, 18, 55, 80, 98 and 108; 2, 18, 55, 80, 98 and 109; 2, 18, 55, 81, 97 and 107; 2, 18, 55, 81, 97 and 108; 2, 18, 55, 81, 97 and 109; 2, 18, 55, 81, 98 and 107; 2, 18, 55, 81, 98 and 108; 2, 18, 55, 81, 98 and 109; 2, 18, 55, 82, 81 and 107; 2, 18, 55, 82, 81 and 108; 2, 18, 55, 82, 81 and 109; 2, 18, 55, 82, 97 and 107; 2, 18, 55, 82, 97 and 108; 2, 18, 55, 82, 97 and 109; 2, 18, 55, 82, 98 and 107; 2, 18, 55, 82, 98 and 108; or 2, 18, 55, 82, 98 and 109.

Also preferably, the vaccine provides combinations of at least six discrete immunization peptides comprising SEQ ID NO:80, more preferably provides immunization polypeptides consisting of amino acids of SEQ ID NOs:1, 11, 53, 80, 81 and 107; 1, 11, 53, 80, 81 and 108; 1, 11, 53, 80, 81 and 109; 1, 11, 53, 80, 97 and 107; 1, 11, 53, 80, 97 and 108; 1, 11, 53, 80, 97 and 109; 1, 11, 53, 80, 98 and 107; 1, 11, 53, 80, 98 and 108; 1, 11, 53, 80, 98 and 109; 2, 11, 53, 80, 81 and 107; 2, 11, 53, 80, 81 and 108; 2, 11, 53, 80, 81 and 109; 2, 11, 53, 80, 97 and 107; 2, 11, 53, 80, 97 and 108; 2, 11, 53, 80, 97 and 109; 2, 11, 53, 80, 98 and 107; 2, 11, 53, 80, 98 and 108; 2, 11, 53, 80, 98 and 109; 1, 13, 53, 80, 81 and 107; 1, 13, 53, 80, 81 and 108; 1, 13, 53, 80, 81 and 109; 1, 13, 53, 80, 97 and 107; 1, 13, 53, 80, 97 and 108; 1, 13, 53, 80, 97 and 109; 1, 13, 53, 80, 98 and 107; 1, 13, 53, 80, 98 and 108; 1, 13, 53, 80, 98 and 109; 2, 13, 53, 80, 81 and 107; 2, 13, 53, 80, 81 and 108; 2, 13, 53, 80, 81 and 109; 2, 13, 53, 80, 97 and 107; 2, 13, 53, 80, 97 and 108; 2, 13, 53, 80, 97 and 109; 2, 13, 53, 80, 98 and 107; 2, 13, 53, 80, 98 and 108; 2, 13, 53, 80, 98 and 109; 1, 14, 53, 80, 81 and 107; 1, 14, 53, 80, 81 and 108; 1, 14, 53, 80, 81 and 109; 1, 14, 53, 80, 97 and 107; 1, 14, 53, 80, 97 and 108; 1, 14, 53, 80, 97 and 109; 1, 14, 53, 80, 98 and 107; 1, 14, 53, 80, 98 and 108; 1, 14, 53, 80, 98 and 109; 2, 14, 53, 80, 81 and 107; 2, 14, 53, 80, 81 and 108; 2, 14, 53, 80, 81 and 109; 2, 14, 53, 80, 97 and 107; 2, 14, 53, 80, 97 and 108; 2, 14, 53, 80, 97 and 109; 2, 14, 53, 80, 98 and 107; 2, 14, 53, 80, 98 and 108; 2, 14, 53, 80, 98 and 109; 1, 17, 53, 80, 81 and 107; 1, 17, 53, 80, 81 and 108; 1, 17, 53, 80, 81 and 109; 1, 17, 53, 80, 97 and 107; 1, 17, 53, 80, 97 and 108; 1, 17, 53, 80, 97 and 109; 1, 17, 53, 80, 98 and 107; 1, 17, 53, 80, 98 and 108; 1, 17, 53, 80, 98 and 109; 2, 17, 53, 80, 81 and 107; 2, 17, 53, 80, 81 and 108; 2, 17, 53, 80, 81 and 109; 2, 17, 53, 80, 97 and 107; 2, 17, 53, 80, 97 and 108; 2, 17, 53, 80, 97 and 109; 2, 17, 53, 80, 98 and 107; 2, 17, 53, 80, 98 and 108; 2, 17, 53, 80, 98 and 109; 1, 18, 53, 80, 81 and 107; 1, 18, 53, 80, 81 and 108; 1, 18, 53, 80, 81 and 109; 1, 18, 53, 80, 97 and 107; 1, 18, 53, 80, 97 and 108; 1, 18, 53, 80, 97 and 109; 1, 18, 53, 80, 98 and 107; 1, 18, 53, 80, 98 and 108; 1, 18, 53, 80, 98 and 109; 2, 18, 53, 80, 81 and 107; 2, 18, 53, 80, 81 and 108; 2, 18, 53, 80, 81 and 109; 2, 18, 53, 80, 97 and 107; 2, 18, 53, 80, 97 and 108; 2, 18, 53, 80, 97 and 109; 2, 18, 53, 80, 98 and 107; 2, 18, 53, 80, 98 and 108; 2, 18, 53, 80, 98 and 109; 1, 11, 54, 80, 81 and 107; 1, 11, 54, 80, 81 and 108; 1, 11, 54, 80, 81 and 109; 1, 11, 54, 80, 97 and 107; 1, 11, 54, 80, 97 and 108; 1, 11, 54, 80, 97 and 109; 1, 11, 54, 80, 98 and 107; 1, 11, 54, 80, 98 and 108; 1, 11, 54, 80, 98 and 109; 2, 11, 54, 80, 81 and 107; 2, 11, 54, 80, 81 and 108; 2, 11, 54, 80, 81 and 109; 2, 11, 54, 80, 97 and 107; 2, 11, 54, 80, 97 and 108; 2, 11, 54, 80, 97 and 109; 2, 11, 54, 80, 98 and 107; 2, 11, 54, 80, 98 and 108; 2, 11, 54, 80, 98 and 109; 1, 13, 54, 80, 81 and 107; 1, 13, 54, 80, 81 and 108; 1, 13, 54, 80, 81 and 109; 1, 13, 54, 80, 97 and 107; 1, 13, 54, 80, 97 and 108; 1, 13, 54, 80, 97 and 109; 1, 13, 54, 80, 98 and 107; 1, 13, 54, 80, 98 and 108; 1, 13, 54, 80, 98 and 109; 2, 13, 54, 80, 81 and 107; 2, 13, 54, 80, 81 and 108; 2, 13, 54, 80, 81 and 109; 2, 13, 54, 80, 97 and 107; 2, 13, 54, 80, 97 and 108; 2, 13, 54, 80, 97 and 109; 2, 13, 54, 80, 98 and 107; 2, 13, 54, 80, 98 and 108; 2, 13, 54, 80, 98 and 109; 1, 14, 54, 80, 81 and 107; 1, 14, 54, 80, 81 and 108; 1, 14, 54, 80, 81 and 109; 1, 14, 54, 80, 97 and 107; 1, 14, 54, 80, 97 and 108; 1, 14, 54, 80, 97 and 109; 1, 14, 54, 80, 98 and 107; 1, 14, 54, 80, 98 and 108; 1, 14, 54, 80, 98 and 109; 2, 14, 54, 80, 81 and 107; 2, 14, 54, 80, 81 and 108; 2, 14, 54, 80, 81 and 109; 2, 14, 54, 80, 97 and 107; 2, 14, 54, 80, 97 and 108; 2, 14, 54, 80, 97 and 109; 2, 14, 54, 80, 98 and 107; 2, 14, 54, 80, 98 and 108; 2, 14, 54, 80, 98 and 109; 1, 17, 54, 80, 81 and 107; 1, 17, 54, 80, 81 and 108; 1, 17, 54, 80, 81 and 109; 1, 17, 54, 80, 97 and 107; 1, 17, 54, 80, 97 and 108; 1, 17, 54, 80, 97 and 109; 1, 17, 54, 80, 98 and 107; 1, 17, 54, 80, 98 and 108; 1, 17, 54, 80, 98 and 109; 2, 17, 54, 80, 81 and 107; 2, 17, 54, 80, 81 and 108; 2, 17, 54, 80, 81 and 109; 2, 17, 54, 80, 97 and 107; 2, 17, 54, 80, 97 and 108; 2, 17, 54, 80, 97 and 109; 2, 17, 54, 80, 98 and 107; 2, 17, 54, 80, 98 and 108; 2, 17, 54, 80, 98 and 109; 1, 18, 54, 80, 81 and 107; 1, 18, 54, 80, 81 and 108; 1, 18, 54, 80, 81 and 109; 1, 18, 54, 80, 97 and 107; 1, 18, 54, 80, 97 and 108; 1, 18, 54, 80, 97 and 109; 1, 18, 54, 80, 98 and 107; 1, 18, 54, 80, 98 and 108; 1, 18, 54, 80, 98 and 109; 2, 18, 54, 80, 81 and 107; 2, 18, 54, 80, 81 and 108; 2, 18, 54, 80, 81 and 109; 2, 18, 54, 80, 97 and 107; 2, 18, 54, 80, 97 and 108; 2, 18, 54, 80, 97 and 109; 2, 18, 54, 80, 98 and 107; 2, 18, 54, 80, 98 and 108; 2, 18, 54, 80, 98 and 109; 1, 11, 55, 80, 81 and 107; 1, 11, 55, 80, 81 and 108; 1, 11, 55, 80, 81 and 109; 1, 11, 55, 80, 97 and 107; 1, 11, 55, 80, 97 and 108; 1, 11, 55, 80, 97 and 109; 1, 11, 55, 80, 98 and 107; 1, 11, 55, 80, 98 and 108; 1, 11, 55, 80, 98 and 109; 2, 11, 55, 80, 81 and 107; 2, 11, 55, 80, 81 and 108; 2, 11, 55, 80, 81 and 109; 2, 11, 55, 80, 97 and 107; 2, 11, 55, 80, 97 and 108; 2, 11, 55, 80, 97 and 109; 2, 11, 55, 80, 98 and 107; 2, 11, 55, 80, 98 and 108; 2, 11, 55, 80, 98 and 109; 1, 13, 55, 80, 81 and 107; 1, 13, 55, 80, 81 and 108; 1, 13, 55, 80, 81 and 109; 1, 13, 55, 80, 97 and 107; 1, 13, 55, 80, 97 and 108; 1, 13, 55, 80, 97 and 109; 1, 13, 55, 80, 98 and 107; 1, 13, 55, 80, 98 and 108; 1, 13, 55, 80, 98 and 109; 2, 13, 55, 80, 81 and 107; 2, 13, 55, 80, 81 and 108; 2, 13, 55, 80, 81 and 109; 2, 13, 55, 80, 97 and 107; 2, 13, 55, 80, 97 and 108; 2, 13, 55, 80, 97 and 109; 2, 13, 55, 80, 98 and 107; 2, 13, 55, 80, 98 and 108; 2, 13, 55, 80, 98 and 109; 1, 14, 55, 80, 81 and 107; 1, 14, 55, 80, 81 and 108; 1, 14, 55, 80, 81 and 109; 1, 14, 55, 80, 97 and 107; 1, 14, 55, 80, 97 and 108; 1, 14, 55, 80, 97 and 109; 1, 14, 55, 80, 98 and 107; 1, 14, 55, 80, 98 and 108; 1, 14, 55, 80, 98 and 109; 2, 14, 55, 80, 81 and 107; 2, 14, 55, 80, 81 and 108; 2, 14, 55, 80, 81 and 109; 2, 14, 55, 80, 97 and 107; 2, 14, 55, 80, 97 and 108; 2, 14, 55, 80, 97 and 109; 2, 14, 55, 80, 98 and 107; 2, 14, 55, 80, 98 and 108; 2, 14, 55, 80, 98 and 109; 1, 17, 55, 80, 81 and 107; 1, 17, 55, 80, 81 and 108; 1, 17, 55, 80, 81 and 109; 1, 17, 55, 80, 97 and 107; 1, 17, 55, 80, 97 and 108; 1, 17, 55, 80, 97 and 109; 1, 17, 55, 80, 98 and 107; 1, 17, 55, 80, 98 and 108; 1, 17, 55, 80, 98 and 109; 2, 17, 55, 80, 81 and 107; 2, 17, 55, 80, 81 and 108; 2, 17, 55, 80, 81 and 109; 2, 17, 55, 80, 97 and 107; 2, 17, 55, 80, 97 and 108; 2, 17, 55, 80, 97 and 109; 2, 17, 55, 80, 98 and 107; 2, 17, 55, 80, 98 and 108; 2, 17, 55, 80, 98 and 109; 1, 18, 55, 80, 81 and 107; 1, 18, 55, 80, 81 and 108; 1, 18, 55, 80, 81 and 109; 1, 18, 55, 80, 97 and 107; 1, 18, 55, 80, 97 and 108; 1, 18, 55, 80, 97 and 109; 1, 18, 55, 80, 98 and 107; 1, 18, 55, 80, 98 and 108; 1, 18, 55, 80, 98 and 109; 2, 18, 55, 80, 81 and 107; 2, 18, 55, 80, 81 and 108; 2, 18, 55, 80, 81 and 109; 2, 18, 55, 80, 97 and 107; 2, 18, 55, 80, 97 and 108; 2, 18, 55, 80, 97 and 109; 2, 18, 55, 80, 98 and 107; 2, 18, 55, 80, 98 and 108; or 2, 18, 55, 80, 98 and 109.

Also preferably, the vaccine provides combinations of at least six discrete immunization peptides comprising SEQ ID NO:81, more preferably provides immunization polypeptides consisting of amino acids of SEQ ID NOs:1, 11, 53, 81, 97 and 107; 1, 11, 53, 81, 97 and 108; 1, 11, 53, 81, 97 and 109; 1, 11, 53, 81, 98 and 107; 1, 11, 53, 81, 98 and 108; 1, 11, 53, 81, 98 and 109; 2, 11, 53, 81, 97 and 107; 2, 11, 53, 81, 97 and 108; 2, 11, 53, 81, 97 and 109; 2, 11, 53, 81, 98 and 107; 2, 11, 53, 81, 98 and 108; 2, 11, 53, 81, 98 and 109; 1, 13, 53, 81, 97 and 107; 1, 13, 53, 81, 97 and 108; 1, 13, 53, 81, 97 and 109; 1, 13, 53, 81, 98 and 107; 1, 13, 53, 81, 98 and 108; 1, 13, 53, 81, 98 and 109; 2, 13, 53, 81, 97 and 107; 2, 13, 53, 81, 97 and 108; 2, 13, 53, 81, 97 and 109; 2, 13, 53, 81, 98 and 107; 2, 13, 53, 81, 98 and 108; 2, 13, 53, 81, 98 and 109; 1, 14, 53, 81, 97 and 107; 1, 14, 53, 81, 97 and 108; 1, 14, 53, 81, 97 and 109; 1, 14, 53, 81, 98 and 107; 1, 14, 53, 81, 98 and 108; 1, 14, 53, 81, 98 and 109; 2, 14, 53, 81, 97 and 107; 2, 14, 53, 81, 97 and 108; 2, 14, 53, 81, 97 and 109; 2, 14, 53, 81, 98 and 107; 2, 14, 53, 81, 98 and 108; 2, 14, 53, 81, 98 and 109; 1, 17, 53, 81, 97 and 107; 1, 17, 53, 81, 97 and 108; 1, 17, 53, 81, 97 and 109; 1, 17, 53, 81, 98 and 107; 1, 17, 53, 81, 98 and 108; 1, 17, 53, 81, 98 and 109; 2, 17, 53, 81, 97 and 107; 2, 17, 53, 81, 97 and 108; 2, 17, 53, 81, 97 and 109; 2, 17, 53, 81, 98 and 107; 2, 17, 53, 81, 98 and 108; 2, 17, 53, 81, 98 and 109; 1, 18, 53, 81, 97 and 107; 1, 18, 53, 81, 97 and 108; 1, 18, 53, 81, 97 and 109; 1, 18, 53, 81, 98 and 107; 1, 18, 53, 81, 98 and 108; 1, 18, 53, 81, 98 and 109; 2, 18, 53, 81, 97 and 107; 2, 18, 53, 81, 97 and 108; 2, 18, 53, 81, 97 and 109; 2, 18, 53, 81, 98 and 107; 2, 18, 53, 81, 98 and 108; 2, 18, 53, 81, 98 and 109; 1, 11, 54, 81, 97 and 107; 1, 11, 54, 81, 97 and 108; 1, 11, 54, 81, 97 and 109; 1, 11, 54, 81, 98 and 107; 1, 11, 54, 81, 98 and 108; 1, 11, 54, 81, 98 and 109; 2, 11, 54, 81, 97 and 107; 2, 11, 54, 81, 97 and 108; 2, 11, 54, 81, 97 and 109; 2, 11, 54, 81, 98 and 107; 2, 11, 54, 81, 98 and 108; 2, 11, 54, 81, 98 and 109; 1, 13, 54, 81, 97 and 107; 1, 13, 54, 81, 97 and 108; 1, 13, 54, 81, 97 and 109; 1, 13, 54, 81, 98 and 107; 1, 13, 54, 81, 98 and 108; 1, 13, 54, 81, 98 and 109; 2, 13, 54, 81, 97 and 107; 2, 13, 54, 81, 97 and 108; 2, 13, 54, 81, 97 and 109; 2, 13, 54, 81, 98 and 107; 2, 13, 54, 81, 98 and 108; 2, 13, 54, 81, 98 and 109; 1, 14, 54, 81, 97 and 107; 1, 14, 54, 81, 97 and 108; 1, 14, 54, 81, 97 and 109; 1, 14, 54, 81, 98 and 107; 1, 14, 54, 81, 98 and 108; 1, 14, 54, 81, 98 and 109; 2, 14, 54, 81, 97 and 107; 2, 14, 54, 81, 97 and 108; 2, 14, 54, 81, 97 and 109; 2, 14, 54, 81, 98 and 107; 2, 14, 54, 81, 98 and 108; 2, 14, 54, 81, 98 and 109; 1, 17, 54, 81, 97 and 107; 1, 17, 54, 81, 97 and 108; 1, 17, 54, 81, 97 and 109; 1, 17, 54, 81, 98 and 107; 1, 17, 54, 81, 98 and 108; 1, 17, 54, 81, 98 and 109; 2, 17, 54, 81, 97 and 107; 2, 17, 54, 81, 97 and 108; 2, 17, 54, 81, 97 and 109; 2, 17, 54, 81, 98 and 107; 2, 17, 54, 81, 98 and 108; 2, 17, 54, 81, 98 and 109; 1, 18, 54, 81, 97 and 107; 1, 18, 54, 81, 97 and 108; 1, 18, 54, 81, 97 and 109; 1, 18, 54, 81, 98 and 107; 1, 18, 54, 81, 98 and 108; 1, 18, 54, 81, 98 and 109; 2, 18, 54, 81, 97 and 107; 2, 18, 54, 81, 97 and 108; 2, 18, 54, 81, 97 and 109; 2, 18, 54, 81, 98 and 107; 2, 18, 54, 81, 98 and 108; 2, 18, 54, 81, 98 and 109; 1, 11, 55, 81, 97 and 107; 1, 11, 55, 81, 97 and 108; 1, 11, 55, 81, 97 and 109; 1, 11, 55, 81, 98 and 107; 1, 11, 55, 81, 98 and 108; 1, 11, 55, 81, 98 and 109; 2, 11, 55, 81, 97 and 107; 2, 11, 55, 81, 97 and 108; 2, 11, 55, 81, 97 and 109; 2, 11, 55, 81, 98 and 107; 2, 11, 55, 81, 98 and 108; 2, 11, 55, 81, 98 and 109; 1, 13, 55, 81, 97 and 107; 1, 13, 55, 81, 97 and 108; 1, 13, 55, 81, 97 and 109; 1, 13, 55, 81, 98 and 107; 1, 13, 55, 81, 98 and 108; 1, 13, 55, 81, 98 and 109; 2, 13, 55, 81, 97 and 107; 2, 13, 55, 81, 97 and 108; 2, 13, 55, 81, 97 and 109; 2, 13, 55, 81, 98 and 107; 2, 13, 55, 81, 98 and 108; 2, 13, 55, 81, 98 and 109; 1, 14, 55, 81, 97 and 107; 1, 14, 55, 81, 97 and 108; 1, 14, 55, 81, 97 and 109; 1, 14, 55, 81, 98 and 107; 1, 14, 55, 81, 98 and 108; 1, 14, 55, 81, 98 and 109; 2, 14, 55, 81, 97 and 107; 2, 14, 55, 81, 97 and 108; 2, 14, 55, 81, 97 and 109; 2, 14, 55, 81, 98 and 107; 2, 14, 55, 81, 98 and 108; 2, 14, 55, 81, 98 and 109; 1, 17, 55, 81, 97 and 107; 1, 17, 55, 81, 97 and 108; 1, 17, 55, 81, 97 and 109; 1, 17, 55, 81, 98 and 107; 1, 17, 55, 81, 98 and 108; 1, 17, 55, 81, 98 and 109; 2, 17, 55, 81, 97 and 107; 2, 17, 55, 81, 97 and 108; 2, 17, 55, 81, 97 and 109; 2, 17, 55, 81, 98 and 107; 2, 17, 55, 81, 98 and 108; 2, 17, 55, 81, 98 and 109; 1, 18, 55, 81, 97 and 107; 1, 18, 55, 81, 97 and 108; 1, 18, 55, 81, 97 and 109; 1, 18, 55, 81, 98 and 107; 1, 18, 55, 81, 98 and 108; 1, 18, 55, 81, 98 and 109; 2, 18, 55, 81, 97 and 107; 2, 18, 55, 81, 97 and 108; 2, 18, 55, 81, 97 and 109; 2, 18, 55, 81, 98 and 107; 2, 18, 55, 81, 98 and 108; 2, 18, 55, 81, 98 and 109; 1, 11, 53, 80, 81 and 107; 1, 11, 53, 80, 81 and 108; 1, 11, 53, 80, 81 and 109; 2, 11, 53, 80, 81 and 107; 2, 11, 53, 80, 81 and 108; 2, 11, 53, 80, 81 and 109; 1, 13, 53, 80, 81 and 107; 1, 13, 53, 80, 81 and 108; 1, 13, 53, 80, 81 and 109; 2, 13, 53, 80, 81 and 107; 2, 13, 53, 80, 81 and 108; 2, 13, 53, 80, 81 and 109; 1, 14, 53, 80, 81 and 107; 1, 14, 53, 80, 81 and 108; 1, 14, 53, 80, 81 and 109; 2, 14, 53, 80, 81 and 107; 2, 14, 53, 80, 81 and 108; 2, 14, 53, 80, 81 and 109; 1, 17, 53, 80, 81 and 107; 1, 17, 53, 80, 81 and 108; 1, 17, 53, 80, 81 and 109; 2, 17, 53, 80, 81 and 107; 2, 17, 53, 80, 81 and 108; 2, 17, 53, 80, 81 and 109; 1, 18, 53, 80, 81 and 107; 1, 18, 53, 80, 81 and 108; 1, 18, 53, 80, 81 and 109; 2, 18, 53, 80, 81 and 107; 2, 18, 53, 80, 81 and 108; 2, 18, 53, 80, 81 and 109; 1, 11, 54, 80, 81 and 107; 1, 11, 54, 80, 81 and 108; 1, 11, 54, 80, 81 and 109; 2, 11, 54, 80, 81 and 107; 2, 11, 54, 80, 81 and 108; 2, 11, 54, 80, 81 and 109; 1, 13, 54, 80, 81 and 107; 1, 13, 54, 80, 81 and 108; 1, 13, 54, 80, 81 and 109; 2, 13, 54, 80, 81 and 107; 2, 13, 54, 80, 81 and 108; 2, 13, 54, 80, 81 and 109; 1, 14, 54, 80, 81 and 107; 1, 14, 54, 80, 81 and 108; 1, 14, 54, 80, 81 and 109; 2, 14, 54, 80, 81 and 107; 2, 14, 54, 80, 81 and 108; 2, 14, 54, 80, 81 and 109; 1, 17, 54, 80, 81 and 107; 1, 17, 54, 80, 81 and 108; 1, 17, 54, 80, 81 and 109; 2, 17, 54, 80, 81 and 107; 2, 17, 54, 80, 81 and 108; 2, 17, 54, 80, 81 and 109; 1, 18, 54, 80, 81 and 107; 1, 18, 54, 80, 81 and 108; 1, 18, 54, 80, 81 and 109; 2, 18, 54, 80, 81 and 107; 2, 18, 54, 80, 81 and 108; 2, 18, 54, 80, 81 and 109; 1, 11, 55, 80, 81 and 107; 1, 11, 55, 80, 81 and 108; 1, 11, 55, 80, 81 and 109; 2, 11, 55, 80, 81 and 107; 2, 11, 55, 80, 81 and 108; 2, 11, 55, 80, 81 and 109; 1, 13, 55, 80, 81 and 107; 1, 13, 55, 80, 81 and 108; 1, 13, 55, 80, 81 and 109; 2, 13, 55, 80, 81 and 107; 2, 13, 55, 80, 81 and 108; 2, 13, 55, 80, 81 and 109; 1, 14, 55, 80, 81 and 107; 1, 14, 55, 80, 81 and 108; 1, 14, 55, 80, 81 and 109; 2, 14, 55, 80, 81 and 107; 2, 14, 55, 80, 81 and 108; 2, 14, 55, 80, 81 and 109; 1, 17, 55, 80, 81 and 107; 1, 17, 55, 80, 81 and 108; 1, 17, 55, 80, 81 and 109; 2, 17, 55, 80, 81 and 107; 2, 17, 55, 80, 81 and 108; 2, 17, 55, 80, 81 and 109; 1, 18, 55, 80, 81 and 107; 1, 18, 55, 80, 81 and 108; 1, 18, 55, 80, 81 and 109; 2, 18, 55, 80, 81 and 107; 2, 18, 55, 80, 81 and 108; 2, 18, 55, 80, 81 and 109; 1, 11, 53, 82, 81 and 107; 1, 11, 53, 82, 81 and 108; 1, 11, 53, 82, 81 and 109; 2, 11, 53, 82, 81 and 107; 2, 11, 53, 82, 81 and 108; 2, 11, 53, 82, 81 and 109; 1, 13, 53, 82, 81 and 107; 1, 13, 53, 82, 81 and 108; 1, 13, 53, 82, 81 and 109; 2, 13, 53, 82, 81 and 107; 2, 13, 53, 82, 81 and 108; 2, 13, 53, 82, 81 and 109; 1, 14, 53, 82, 81 and 107; 1, 14, 53, 82, 81 and 108; 1, 14, 53, 82, 81 and 109; 2, 14, 53, 82, 81 and 107; 2, 14, 53, 82, 81 and 108; 2, 14, 53, 82, 81 and 109; 1, 17, 53, 82, 81 and 107; 1, 17, 53, 82, 81 and 108; 1, 17, 53, 82, 81 and 109; 2, 17, 53, 82, 81 and 107; 2, 17, 53, 82, 81 and 108; 2, 17, 53, 82, 81 and 109; 1, 18, 53, 82, 81 and 107; 1, 18, 53, 82, 81 and 108; 1, 18, 53, 82, 81 and 109; 2, 18, 53, 82, 81 and 107; 2, 18, 53, 82, 81 and 108; 2, 18, 53, 82, 81 and 109; 1, 11, 54, 82, 81 and 107; 1, 11, 54, 82, 81 and 108; 1, 11, 54, 82, 81 and 109; 2, 11, 54, 82, 81 and 107; 2, 11, 54, 82, 81 and 108; 2, 11, 54, 82, 81 and 109; 1, 13, 54, 82, 81 and 107; 1, 13, 54, 82, 81 and 108; 1, 13, 54, 82, 81 and 109; 2, 13, 54, 82, 81 and 107; 2, 13, 54, 82, 81 and 108; 2, 13, 54, 82, 81 and 109; 1, 14, 54, 82, 81 and 107; 1, 14, 54, 82, 81 and 108; 1, 14, 54, 82, 81 and 109; 2, 14, 54, 82, 81 and 107; 2, 14, 54, 82, 81 and 108; 2, 14, 54, 82, 81 and 109; 1, 17, 54, 82, 81 and 107; 1, 17, 54, 82, 81 and 108; 1, 17, 54, 82, 81 and 109; 2, 17, 54, 82, 81 and 107; 2, 17, 54, 82, 81 and 108; 2, 17, 54, 82, 81 and 109; 1, 18, 54, 82, 81 and 107; 1, 18, 54, 82, 81 and 108; 1, 18, 54, 82, 81 and 109; 2, 18, 54, 82, 81 and 107; 2, 18, 54, 82, 81 and 108; 2, 18, 54, 82, 81 and 109; 1, 11, 55, 82, 81 and 107; 1, 11, 55, 82, 81 and 108; 1, 11, 55, 82, 81 and 109; 2, 11, 55, 82, 81 and 107; 2, 11, 55, 82, 81 and 108; 2, 11, 55, 82, 81 and 109; 1, 13, 55, 82, 81 and 107; 1, 13, 55, 82, 81 and 108; 1, 13, 55, 82, 81 and 109; 2, 13, 55, 82, 81 and 107; 2, 13, 55, 82, 81 and 108; 2, 13, 55, 82, 81 and 109; 1, 14, 55, 82, 81 and 107; 1, 14, 55, 82, 81 and 108; 1, 14, 55, 82, 81 and 109; 2, 14, 55, 82, 81 and 107; 2, 14, 55, 82, 81 and 108; 2, 14, 55, 82, 81 and 109; 1, 17, 55, 82, 81 and 107; 1, 17, 55, 82, 81 and 108; 1, 17, 55, 82, 81 and 109; 2, 17, 55, 82, 81 and 107; 2, 17, 55, 82, 81 and 108; 2, 17, 55, 82, 81 and 109; 1, 18, 55, 82, 81 and 107; 1, 18, 55, 82, 81 and 108; 1, 18, 55, 82, 81 and 109; 2, 18, 55, 82, 81 and 107; 2, 18, 55, 82, 81 and 108; or 2, 18, 55, 82, 81 and 109.

Also preferably, the vaccine provides combinations of at least six discrete immunization peptides comprising SEQ ID NO:82, more preferably provides immunization polypeptides consisting of amino acids of SEQ ID NOs:1, 11, 53, 82, 81 and 107; 1, 11, 53, 82, 81 and 108; 1, 11, 53, 82, 81 and 109; 1, 11, 53, 82, 97 and 107; 1, 11, 53, 82, 97 and 108; 1, 11, 53, 82, 97 and 109; 1, 11, 53, 82, 98 and 107; 1, 11, 53, 82, 98 and 108; 1, 11, 53, 82, 98 and 109; 2, 11, 53, 82, 81 and 107; 2, 11, 53, 82, 81 and 108; 2, 11, 53, 82, 81 and 109; 2, 11, 53, 82, 97 and 107; 2, 11, 53, 82, 97 and 108; 2, 11, 53, 82, 97 and 109; 2, 11, 53, 82, 98 and 107; 2, 11, 53, 82, 98 and 108; 2, 11, 53, 82, 98 and 109; 1, 13, 53, 82, 81 and 107; 1, 13, 53, 82, 81 and 108; 1, 13, 53, 82, 81 and 109; 1, 13, 53, 82, 97 and 107; 1, 13, 53, 82, 97 and 108; 1, 13, 53, 82, 97 and 109; 1, 13, 53, 82, 98 and 107; 1, 13, 53, 82, 98 and 108; 1, 13, 53, 82, 98 and 109; 2, 13, 53, 82, 81 and 107; 2, 13, 53, 82, 81 and 108; 2, 13, 53, 82, 81 and 109; 2, 13, 53, 82, 97 and 107; 2, 13, 53, 82, 97 and 108; 2, 13, 53, 82, 97 and 109; 2, 13, 53, 82, 98 and 107; 2, 13, 53, 82, 98 and 108; 2, 13, 53, 82, 98 and 109; 1, 14, 53, 82, 81 and 107; 1, 14, 53, 82, 81 and 108; 1, 14, 53, 82, 81 and 109; 1, 14, 53, 82, 97 and 107; 1, 14, 53, 82, 97 and 108; 1, 14, 53, 82, 97 and 109; 1, 14, 53, 82, 98 and 107; 1, 14, 53, 82, 98 and 108; 1, 14, 53, 82, 98 and 109; 2, 14, 53, 82, 81 and 107; 2, 14, 53, 82, 81 and 108; 2, 14, 53, 82, 81 and 109; 2, 14, 53, 82, 97 and 107; 2, 14, 53, 82, 97 and 108; 2, 14, 53, 82, 97 and 109; 2, 14, 53, 82, 98 and 107; 2, 14, 53, 82, 98 and 108; 2, 14, 53, 82, 98 and 109; 1, 17, 53, 82, 81 and 107; 1, 17, 53, 82, 81 and 108; 1, 17, 53, 82, 81 and 109; 1, 17, 53, 82, 97 and 107; 1, 17, 53, 82, 97 and 108; 1, 17, 53, 82, 97 and 109; 1, 17, 53, 82, 98 and 107; 1, 17, 53, 82, 98 and 108; 1, 17, 53, 82, 98 and 109; 2, 17, 53, 82, 81 and 107; 2, 17, 53, 82, 81 and 108; 2, 17, 53, 82, 81 and 109; 2, 17, 53, 82, 97 and 107; 2, 17, 53, 82, 97 and 108; 2, 17, 53, 82, 97 and 109; 2, 17, 53, 82, 98 and 107; 2, 17, 53, 82, 98 and 108; 2, 17, 53, 82, 98 and 109; 1, 18, 53, 82, 81 and 107; 1, 18, 53, 82, 81 and 108; 1, 18, 53, 82, 81 and 109; 1, 18, 53, 82, 97 and 107; 1, 18, 53, 82, 97 and 108; 1, 18, 53, 82, 97 and 109; 1, 18, 53, 82, 98 and 107; 1, 18, 53, 82, 98 and 108; 1, 18, 53, 82, 98 and 109; 2, 18, 53, 82, 81 and 107; 2, 18, 53, 82, 81 and 108; 2, 18, 53, 82, 81 and 109; 2, 18, 53, 82, 97 and 107; 2, 18, 53, 82, 97 and 108; 2, 18, 53, 82, 97 and 109; 2, 18, 53, 82, 98 and 107; 2, 18, 53, 82, 98 and 108; 2, 18, 53, 82, 98 and 109; 1, 11, 54, 82, 81 and 107; 1, 11, 54, 82, 81 and 108; 1, 11, 54, 82, 81 and 109; 1, 11, 54, 82, 97 and 107; 1, 11, 54, 82, 97 and 108; 1, 11, 54, 82, 97 and 109; 1, 11, 54, 82, 98 and 107; 1, 11, 54, 82, 98 and 108; 1, 11, 54, 82, 98 and 109; 2, 11, 54, 82, 81 and 107; 2, 11, 54, 82, 81 and 108; 2, 11, 54, 82, 81 and 109; 2, 11, 54, 82, 97 and 107; 2, 11, 54, 82, 97 and 108; 2, 11, 54, 82, 97 and 109; 2, 11, 54, 82, 98 and 107; 2, 11, 54, 82, 98 and 108; 2, 11, 54, 82, 98 and 109; 1, 13, 54, 82, 81 and 107; 1, 13, 54, 82, 81 and 108; 1, 13, 54, 82, 81 and 109; 1, 13, 54, 82, 97 and 107; 1, 13, 54, 82, 97 and 108; 1, 13, 54, 82, 97 and 109; 1, 13, 54, 82, 98 and 107; 1, 13, 54, 82, 98 and 108; 1, 13, 54, 82, 98 and 109; 2, 13, 54, 82, 81 and 107; 2, 13, 54, 82, 81 and 108; 2, 13, 54, 82, 81 and 109; 2, 13, 54, 82, 97 and 107; 2, 13, 54, 82, 97 and 108; 2, 13, 54, 82, 97 and 109; 2, 13, 54, 82, 98 and 107; 2, 13, 54, 82, 98 and 108; 2, 13, 54, 82, 98 and 109; 1, 14, 54, 82, 81 and 107; 1, 14, 54, 82, 81 and 108; 1, 14, 54, 82, 81 and 109; 1, 14, 54, 82, 97 and 107; 1, 14, 54, 82, 97 and 108; 1, 14, 54, 82, 97 and 109; 1, 14, 54, 82, 98 and 107; 1, 14, 54, 82, 98 and 108; 1, 14, 54, 82, 98 and 109; 2, 14, 54, 82, 81 and 107; 2, 14, 54, 82, 81 and 108; 2, 14, 54, 82, 81 and 109; 2, 14, 54, 82, 97 and 107; 2, 14, 54, 82, 97 and 108; 2, 14, 54, 82, 97 and 109; 2, 14, 54, 82, 98 and 107; 2, 14, 54, 82, 98 and 108; 2, 14, 54, 82, 98 and 109; 1, 17, 54, 82, 81 and 107; 1, 17, 54, 82, 81 and 108; 1, 17, 54, 82, 81 and 109; 1, 17, 54, 82, 97 and 107; 1, 17, 54, 82, 97 and 108; 1, 17, 54, 82, 97 and 109; 1, 17, 54, 82, 98 and 107; 1, 17, 54, 82, 98 and 108; 1, 17, 54, 82, 98 and 109; 2, 17, 54, 82, 81 and 107; 2, 17, 54, 82, 81 and 108; 2, 17, 54, 82, 81 and 109; 2, 17, 54, 82, 97 and 107; 2, 17, 54, 82, 97 and 108; 2, 17, 54, 82, 97 and 109; 2, 17, 54, 82, 98 and 107; 2, 17, 54, 82, 98 and 108; 2, 17, 54, 82, 98 and 109; 1, 18, 54, 82, 81 and 107; 1, 18, 54, 82, 81 and 108; 1, 18, 54, 82, 81 and 109; 1, 18, 54, 82, 97 and 107; 1, 18, 54, 82, 97 and 108; 1, 18, 54, 82, 97 and 109; 1, 18, 54, 82, 98 and 107; 1, 18, 54, 82, 98 and 108; 1, 18, 54, 82, 98 and 109; 2, 18, 54, 82, 81 and 107; 2, 18, 54, 82, 81 and 108; 2, 18, 54, 82, 81 and 109; 2, 18, 54, 82, 97 and 107; 2, 18, 54, 82, 97 and 108; 2, 18, 54, 82, 97 and 109; 2, 18, 54, 82, 98 and 107; 2, 18, 54, 82, 98 and 108; 2, 18, 54, 82, 98 and 109; 1, 11, 55, 82, 81 and 107; 1, 11, 55, 82, 81 and 108; 1, 11, 55, 82, 81 and 109; 1, 11, 55, 82, 97 and 107; 1, 11, 55, 82, 97 and 108; 1, 11, 55, 82, 97 and 109; 1, 11, 55, 82, 98 and 107; 1, 11, 55, 82, 98 and 108; 1, 11, 55, 82, 98 and 109; 2, 11, 55, 82, 81 and 107; 2, 11, 55, 82, 81 and 108; 2, 11, 55, 82, 81 and 109; 2, 11, 55, 82, 97 and 107; 2, 11, 55, 82, 97 and 108; 2, 11, 55, 82, 97 and 109; 2, 11, 55, 82, 98 and 107; 2, 11, 55, 82, 98 and 108; 2, 11, 55, 82, 98 and 109; 1, 13, 55, 82, 81 and 107; 1, 13, 55, 82, 81 and 108; 1, 13, 55, 82, 81 and 109; 1, 13, 55, 82, 97 and 107; 1, 13, 55, 82, 97 and 108; 1, 13, 55, 82, 97 and 109; 1, 13, 55, 82, 98 and 107; 1, 13, 55, 82, 98 and 108; 1, 13, 55, 82, 98 and 109; 2, 13, 55, 82, 81 and 107; 2, 13, 55, 82, 81 and 108; 2, 13, 55, 82, 81 and 109; 2, 13, 55, 82, 97 and 107; 2, 13, 55, 82, 97 and 108; 2, 13, 55, 82, 97 and 109; 2, 13, 55, 82, 98 and 107; 2, 13, 55, 82, 98 and 108; 2, 13, 55, 82, 98 and 109; 1, 14, 55, 82, 81 and 107; 1, 14, 55, 82, 81 and 108; 1, 14, 55, 82, 81 and 109; 1, 14, 55, 82, 97 and 107; 1, 14, 55, 82, 97 and 108; 1, 14, 55, 82, 97 and 109; 1, 14, 55, 82, 98 and 107; 1, 14, 55, 82, 98 and 108; 1, 14, 55, 82, 98 and 109; 2, 14, 55, 82, 81 and 107; 2, 14, 55, 82, 81 and 108; 2, 14, 55, 82, 81 and 109; 2, 14, 55, 82, 97 and 107; 2, 14, 55, 82, 97 and 108; 2, 14, 55, 82, 97 and 109; 2, 14, 55, 82, 98 and 107; 2, 14, 55, 82, 98 and 108; 2, 14, 55, 82, 98 and 109; 1, 17, 55, 82, 81 and 107; 1, 17, 55, 82, 81 and 108; 1, 17, 55, 82, 81 and 109; 1, 17, 55, 82, 97 and 107; 1, 17, 55, 82, 97 and 108; 1, 17, 55, 82, 97 and 109; 1, 17, 55, 82, 98 and 107; 1, 17, 55, 82, 98 and 108; 1, 17, 55, 82, 98 and 109; 2, 17, 55, 82, 81 and 107; 2, 17, 55, 82, 81 and 108; 2, 17, 55, 82, 81 and 109; 2, 17, 55, 82, 97 and 107; 2, 17, 55, 82, 97 and 108; 2, 17, 55, 82, 97 and 109; 2, 17, 55, 82, 98 and 107; 2, 17, 55, 82, 98 and 108; 2, 17, 55, 82, 98 and 109; 1, 18, 55, 82, 81 and 107; 1, 18, 55, 82, 81 and 108; 1, 18, 55, 82, 81 and 109; 1, 18, 55, 82, 97 and 107; 1, 18, 55, 82, 97 and 108; 1, 18, 55, 82, 97 and 109; 1, 18, 55, 82, 98 and 107; 1, 18, 55, 82, 98 and 108; 1, 18, 55, 82, 98 and 109; 2, 18, 55, 82, 81 and 107; 2, 18, 55, 82, 81 and 108; 2, 18, 55, 82, 81 and 109; 2, 18, 55, 82, 97 and 107; 2, 18, 55, 82, 97 and 108; 2, 18, 55, 82, 97 and 109; 2, 18, 55, 82, 98 and 107; 2, 18, 55, 82, 98 and 108; or 2, 18, 55, 82, 98 and 109.

Also preferably, the vaccine provides combinations of at least six discrete immunization peptides comprising SEQ ID NO:97, more preferably provides immunization polypeptides consisting of amino acids of SEQ ID NOs:1, 11, 53, 80, 97 and 107; 1, 11, 53, 80, 97 and 108; 1, 11, 53, 80, 97 and 109; 2, 11, 53, 80, 97 and 107; 2, 11, 53, 80, 97 and 108; 2, 11, 53, 80, 97 and 109; 1, 13, 53, 80, 97 and 107; 1, 13, 53, 80, 97 and 108; 1, 13, 53, 80, 97 and 109; 2, 13, 53, 80, 97 and 107; 2, 13, 53, 80, 97 and 108; 2, 13, 53, 80, 97 and 109; 1, 14, 53, 80, 97 and 107; 1, 14, 53, 80, 97 and 108; 1, 14, 53, 80, 97 and 109; 2, 14, 53, 80, 97 and 107; 2, 14, 53, 80, 97 and 108; 2, 14, 53, 80, 97 and 109; 1, 17, 53, 80, 97 and 107; 1, 17, 53, 80, 97 and 108; 1, 17, 53, 80, 97 and 109; 2, 17, 53, 80, 97 and 107; 2, 17, 53, 80, 97 and 108; 2, 17, 53, 80, 97 and 109; 1, 18, 53, 80, 97 and 107; 1, 18, 53, 80, 97 and 108; 1, 18, 53, 80, 97 and 109; 2, 18, 53, 80, 97 and 107; 2, 18, 53, 80, 97 and 108; 2, 18, 53, 80, 97 and 109; 1, 11, 54, 80, 97 and 107; 1, 11, 54, 80, 97 and 108; 1, 11, 54, 80, 97 and 109; 2, 11, 54, 80, 97 and 107; 2, 11, 54, 80, 97 and 108; 2, 11, 54, 80, 97 and 109; 1, 13, 54, 80, 97 and 107; 1, 13, 54, 80, 97 and 108; 1, 13, 54, 80, 97 and 109; 2, 13, 54, 80, 97 and 107; 2, 13, 54, 80, 97 and 108; 2, 13, 54, 80, 97 and 109; 1, 14, 54, 80, 97 and 107; 1, 14, 54, 80, 97 and 108; 1, 14, 54, 80, 97 and 109; 2, 14, 54, 80, 97 and 107; 2, 14, 54, 80, 97 and 108; 2, 14, 54, 80, 97 and 109; 1, 17, 54, 80, 97 and 107; 1, 17, 54, 80, 97 and 108; 1, 17, 54, 80, 97 and 109; 2, 17, 54, 80, 97 and 107; 2, 17, 54, 80, 97 and 108; 2, 17, 54, 80, 97 and 109; 1, 18, 54, 80, 97 and 107; 1, 18, 54, 80, 97 and 108; 1, 18, 54, 80, 97 and 109; 2, 18, 54, 80, 97 and 107; 2, 18, 54, 80, 97 and 108; 2, 18, 54, 80, 97 and 109; 1, 11, 55, 80, 97 and 107; 1, 11, 55, 80, 97 and 108; 1, 11, 55, 80, 97 and 109; 2, 11, 55, 80, 97 and 107; 2, 11, 55, 80, 97 and 108; 2, 11, 55, 80, 97 and 109; 1, 13, 55, 80, 97 and 107; 1, 13, 55, 80, 97 and 108; 1, 13, 55, 80, 97 and 109; 2, 13, 55, 80, 97 and 107; 2, 13, 55, 80, 97 and 108; 2, 13, 55, 80, 97 and 109; 1, 14, 55, 80, 97 and 107; 1, 14, 55, 80, 97 and 108; 1, 14, 55, 80, 97 and 109; 2, 14, 55, 80, 97 and 107; 2, 14, 55, 80, 97 and 108; 2, 14, 55, 80, 97 and 109; 1, 17, 55, 80, 97 and 107; 1, 17, 55, 80, 97 and 108; 1, 17, 55, 80, 97 and 109; 2, 17, 55, 80, 97 and 107; 2, 17, 55, 80, 97 and 108; 2, 17, 55, 80, 97 and 109; 1, 18, 55, 80, 97 and 107; 1, 18, 55, 80, 97 and 108; 1, 18, 55, 80, 97 and 109; 2, 18, 55, 80, 97 and 107; 2, 18, 55, 80, 97 and 108; 2, 18, 55, 80, 97 and 109; 1, 11, 53, 81, 97 and 107; 1, 11, 53, 81, 97 and 108; 1, 11, 53, 81, 97 and 109; 2, 11, 53, 81, 97 and 107; 2, 11, 53, 81, 97 and 108; 2, 11, 53, 81, 97 and 109; 1, 13, 53, 81, 97 and 107; 1, 13, 53, 81, 97 and 108; 1, 13, 53, 81, 97 and 109; 2, 13, 53, 81, 97 and 107; 2, 13, 53, 81, 97 and 108; 2, 13, 53, 81, 97 and 109; 1, 14, 53, 81, 97 and 107; 1, 14, 53, 81, 97 and 108; 1, 14, 53, 81, 97 and 109; 2, 14, 53, 81, 97 and 107; 2, 14, 53, 81, 97 and 108; 2, 14, 53, 81, 97 and 109; 1, 17, 53, 81, 97 and 107; 1, 17, 53, 81, 97 and 108; 1, 17, 53, 81, 97 and 109; 2, 17, 53, 81, 97 and 107; 2, 17, 53, 81, 97 and 108; 2, 17, 53, 81, 97 and 109; 1, 18, 53, 81, 97 and 107; 1, 18, 53, 81, 97 and 108; 1, 18, 53, 81, 97 and 109; 2, 18, 53, 81, 97 and 107; 2, 18, 53, 81, 97 and 108; 2, 18, 53, 81, 97 and 109; 1, 11, 54, 81, 97 and 107; 1, 11, 54, 81, 97 and 108; 1, 11, 54, 81, 97 and 109; 2, 11, 54, 81, 97 and 107; 2, 11, 54, 81, 97 and 108; 2, 11, 54, 81, 97 and 109; 1, 13, 54, 81, 97 and 107; 1, 13, 54, 81, 97 and 108; 1, 13, 54, 81, 97 and 109; 2, 13, 54, 81, 97 and 107; 2, 13, 54, 81, 97 and 108; 2, 13, 54, 81, 97 and 109; 1, 14, 54, 81, 97 and 107; 1, 14, 54, 81, 97 and 108; 1, 14, 54, 81, 97 and 109; 2, 14, 54, 81, 97 and 107; 2, 14, 54, 81, 97 and 108; 2, 14, 54, 81, 97 and 109; 1, 17, 54, 81, 97 and 107; 1, 17, 54, 81, 97 and 108; 1, 17, 54, 81, 97 and 109; 2, 17, 54, 81, 97 and 107; 2, 17, 54, 81, 97 and 108; 2, 17, 54, 81, 97 and 109; 1, 18, 54, 81, 97 and 107; 1, 18, 54, 81, 97 and 108; 1, 18, 54, 81, 97 and 109; 2, 18, 54, 81, 97 and 107; 2, 18, 54, 81, 97 and 108; 2, 18, 54, 81, 97 and 109; 1, 11, 55, 81, 97 and 107; 1, 11, 55, 81, 97 and 108; 1, 11, 55, 81, 97 and 109; 2, 11, 55, 81, 97 and 107; 2, 11, 55, 81, 97 and 108; 2, 11, 55, 81, 97 and 109; 1, 13, 55, 81, 97 and 107; 1, 13, 55, 81, 97 and 108; 1, 13, 55, 81, 97 and 109; 2, 13, 55, 81, 97 and 107; 2, 13, 55, 81, 97 and 108; 2, 13, 55, 81, 97 and 109; 1, 14, 55, 81, 97 and 107; 1, 14, 55, 81, 97 and 108; 1, 14, 55, 81, 97 and 109; 2, 14, 55, 81, 97 and 107; 2, 14, 55, 81, 97 and 108; 2, 14, 55, 81, 97 and 109; 1, 17, 55, 81, 97 and 107; 1, 17, 55, 81, 97 and 108; 1, 17, 55, 81, 97 and 109; 2, 17, 55, 81, 97 and 107; 2, 17, 55, 81, 97 and 108; 2, 17, 55, 81, 97 and 109; 1, 18, 55, 81, 97 and 107; 1, 18, 55, 81, 97 and 108; 1, 18, 55, 81, 97 and 109; 2, 18, 55, 81, 97 and 107; 2, 18, 55, 81, 97 and 108; 2, 18, 55, 81, 97 and 109; 1, 11, 53, 82, 97 and 107; 1, 11, 53, 82, 97 and 108; 1, 11, 53, 82, 97 and 109; 2, 11, 53, 82, 97 and 107; 2, 11, 53, 82, 97 and 108; 2, 11, 53, 82, 97 and 109; 1, 13, 53, 82, 97 and 107; 1, 13, 53, 82, 97 and 108; 1, 13, 53, 82, 97 and 109; 2, 13, 53, 82, 97 and 107; 2, 13, 53, 82, 97 and 108; 2, 13, 53, 82, 97 and 109; 1, 14, 53, 82, 97 and 107; 1, 14, 53, 82, 97 and 108; 1, 14, 53, 82, 97 and 109; 2, 14, 53, 82, 97 and 107; 2, 14, 53, 82, 97 and 108; 2, 14, 53, 82, 97 and 109; 1, 17, 53, 82, 97 and 107; 1, 17, 53, 82, 97 and 108; 1, 17, 53, 82, 97 and 109; 2, 17, 53, 82, 97 and 107; 2, 17, 53, 82, 97 and 108; 2, 17, 53, 82, 97 and 109; 1, 18, 53, 82, 97 and 107; 1, 18, 53, 82, 97 and 108; 1, 18, 53, 82, 97 and 109; 2, 18, 53, 82, 97 and 107; 2, 18, 53, 82, 97 and 108; 2, 18, 53, 82, 97 and 109; 1, 11, 54, 82, 97 and 107; 1, 11, 54, 82, 97 and 108; 1, 11, 54, 82, 97 and 109; 2, 11, 54, 82, 97 and 107; 2, 11, 54, 82, 97 and 108; 2, 11, 54, 82, 97 and 109; 1, 13, 54, 82, 97 and 107; 1, 13, 54, 82, 97 and 108; 1, 13, 54, 82, 97 and 109; 2, 13, 54, 82, 97 and 107; 2, 13, 54, 82, 97 and 108; 2, 13, 54, 82, 97 and 109; 1, 14, 54, 82, 97 and 107; 1, 14, 54, 82, 97 and 108; 1, 14, 54, 82, 97 and 109; 2, 14, 54, 82, 97 and 107; 2, 14, 54, 82, 97 and 108; 2, 14, 54, 82, 97 and 109; 1, 17, 54, 82, 97 and 107; 1, 17, 54, 82, 97 and 108; 1, 17, 54, 82, 97 and 109; 2, 17, 54, 82, 97 and 107; 2, 17, 54, 82, 97 and 108; 2, 17, 54, 82, 97 and 109; 1, 18, 54, 82, 97 and 107; 1, 18, 54, 82, 97 and 108; 1, 18, 54, 82, 97 and 109; 2, 18, 54, 82, 97 and 107; 2, 18, 54, 82, 97 and 108; 2, 18, 54, 82, 97 and 109; 1, 11, 55, 82, 97 and 107; 1, 11, 55, 82, 97 and 108; 1, 11, 55, 82, 97 and 109; 2, 11, 55, 82, 97 and 107; 2, 11, 55, 82, 97 and 108; 2, 11, 55, 82, 97 and 109; 1, 13, 55, 82, 97 and 107; 1, 13, 55, 82, 97 and 108; 1, 13, 55, 82, 97 and 109; 2, 13, 55, 82, 97 and 107; 2, 13, 55, 82, 97 and 108; 2, 13, 55, 82, 97 and 109; 1, 14, 55, 82, 97 and 107; 1, 14, 55, 82, 97 and 108; 1, 14, 55, 82, 97 and 109; 2, 14, 55, 82, 97 and 107; 2, 14, 55, 82, 97 and 108; 2, 14, 55, 82, 97 and 109; 1, 17, 55, 82, 97 and 107; 1, 17, 55, 82, 97 and 108; 1, 17, 55, 82, 97 and 109; 2, 17, 55, 82, 97 and 107; 2, 17, 55, 82, 97 and 108; 2, 17, 55, 82, 97 and 109; 1, 18, 55, 82, 97 and 107; 1, 18, 55, 82, 97 and 108; 1, 18, 55, 82, 97 and 109; 2, 18, 55, 82, 97 and 107; 2, 18, 55, 82, 97 and 108; or 2, 18, 55, 82, 97 and 109.

Also preferably, the vaccine provides combinations of at least six discrete immunization peptides comprising SEQ ID NO:98, more preferably provides immunization polypeptides consisting of amino acids of SEQ ID NOs: 1, 11, 53, 80, 98 and 107; 1, 11, 53, 80, 98 and 108; 1, 11, 53, 80, 98 and 109; 2, 11, 53, 80, 98 and 107; 2, 11, 53, 80, 98 and 108; 2, 11, 53, 80, 98 and 109; 1, 13, 53, 80, 98 and 107; 1, 13, 53, 80, 98 and 108; 1, 13, 53, 80, 98 and 109; 2, 13, 53, 80, 98 and 107; 2, 13, 53, 80, 98 and 108; 2, 13, 53, 80, 98 and 109; 1, 14, 53, 80, 98 and 107; 1, 14, 53, 80, 98 and 108; 1, 14, 53, 80, 98 and 109; 2, 14, 53, 80, 98 and 107; 2, 14, 53, 80, 98 and 108; 2, 14, 53, 80, 98 and 109; 1, 17, 53, 80, 98 and 107; 1, 17, 53, 80, 98 and 108; 1, 17, 53, 80, 98 and 109; 2, 17, 53, 80, 98 and 107; 2, 17, 53, 80, 98 and 108; 2, 17, 53, 80, 98 and 109; 1, 18, 53, 80, 98 and 107; 1, 18, 53, 80, 98 and 108; 1, 18, 53, 80, 98 and 109; 2, 18, 53, 80, 98 and 107; 2, 18, 53, 80, 98 and 108; 2, 18, 53, 80, 98 and 109; 1, 11, 54, 80, 98 and 107; 1, 11, 54, 80, 98 and 108; 1, 11, 54, 80, 98 and 109; 2, 11, 54, 80, 98 and 107; 2, 11, 54, 80, 98 and 108; 2, 11, 54, 80, 98 and 109; 1, 13, 54, 80, 98 and 107; 1, 13, 54, 80, 98 and 108; 1, 13, 54, 80, 98 and 109; 2, 13, 54, 80, 98 and 107; 2, 13, 54, 80, 98 and 108; 2, 13, 54, 80, 98 and 109; 1, 14, 54, 80, 98 and 107; 1, 14, 54, 80, 98 and 108; 1, 14, 54, 80, 98 and 109; 2, 14, 54, 80, 98 and 107; 2, 14, 54, 80, 98 and 108; 2, 14, 54, 80, 98 and 109; 1, 17, 54, 80, 98 and 107; 1, 17, 54, 80, 98 and 108; 1, 17, 54, 80, 98 and 109; 2, 17, 54, 80, 98 and 107; 2, 17, 54, 80, 98 and 108; 2, 17, 54, 80, 98 and 109; 1, 18, 54, 80, 98 and 107; 1, 18, 54, 80, 98 and 108; 1, 18, 54, 80, 98 and 109; 2, 18, 54, 80, 98 and 107; 2, 18, 54, 80, 98 and 108; 2, 18, 54, 80, 98 and 109; 1, 11, 55, 80, 98 and 107; 1, 11, 55, 80, 98 and 108; 1, 11, 55, 80, 98 and 109; 2, 11, 55, 80, 98 and 107; 2, 11, 55, 80, 98 and 108; 2, 11, 55, 80, 98 and 109; 1, 13, 55, 80, 98 and 107; 1, 13, 55, 80, 98 and 108; 1, 13, 55, 80, 98 and 109; 2, 13, 55, 80, 98 and 107; 2, 13, 55, 80, 98 and 108; 2, 13, 55, 80, 98 and 109; 1, 14, 55, 80, 98 and 107; 1, 14, 55, 80, 98 and 108; 1, 14, 55, 80, 98 and 109; 2, 14, 55, 80, 98 and 107; 2, 14, 55, 80, 98 and 108; 2, 14, 55, 80, 98 and 109; 1, 17, 55, 80, 98 and 107; 1, 17, 55, 80, 98 and 108; 1, 17, 55, 80, 98 and 109; 2, 17, 55, 80, 98 and 107; 2, 17, 55, 80, 98 and 108; 2, 17, 55, 80, 98 and 109; 1, 18, 55, 80, 98 and 107; 1, 18, 55, 80, 98 and 108; 1, 18, 55, 80, 98 and 109; 2, 18, 55, 80, 98 and 107; 2, 18, 55, 80, 98 and 108; 2, 18, 55, 80, 98 and 109; 1, 11, 53, 81, 98 and 107; 1, 11, 53, 81, 98 and 108; 1, 11, 53, 81, 98 and 109; 2, 11, 53, 81, 98 and 107; 2, 11, 53, 81, 98 and 108; 2, 11, 53, 81, 98 and 109; 1, 13, 53, 81, 98 and 107; 1, 13, 53, 81, 98 and 108; 1, 13, 53, 81, 98 and 109; 2, 13, 53, 81, 98 and 107; 2, 13, 53, 81, 98 and 108; 2, 13, 53, 81, 98 and 109; 1, 14, 53, 81, 98 and 107; 1, 14, 53, 81, 98 and 108; 1, 14, 53, 81, 98 and 109; 2, 14, 53, 81, 98 and 107; 2, 14, 53, 81, 98 and 108; 2, 14, 53, 81, 98 and 109; 1, 17, 53, 81, 98 and 107; 1, 17, 53, 81, 98 and 108; 1, 17, 53, 81, 98 and 109; 2, 17, 53, 81, 98 and 107; 2, 17, 53, 81, 98 and 108; 2, 17, 53, 81, 98 and 109; 1, 18, 53, 81, 98 and 107; 1, 18, 53, 81, 98 and 108; 1, 18, 53, 81, 98 and 109; 2, 18, 53, 81, 98 and 107; 2, 18, 53, 81, 98 and 108; 2, 18, 53, 81, 98 and 109; 1, 11, 54, 81, 98 and 107; 1, 11, 54, 81, 98 and 108; 1, 11, 54, 81, 98 and 109; 2, 11, 54, 81, 98 and 107; 2, 11, 54, 81, 98 and 108; 2, 11, 54, 81, 98 and 109; 1, 13, 54, 81, 98 and 107; 1, 13, 54, 81, 98 and 108; 1, 13, 54, 81, 98 and 109; 2, 13, 54, 81, 98 and 107; 2, 13, 54, 81, 98 and 108; 2, 13, 54, 81, 98 and 109; 1, 14, 54, 81, 98 and 107; 1, 14, 54, 81, 98 and 108; 1, 14, 54, 81, 98 and 109; 2, 14, 54, 81, 98 and 107; 2, 14, 54, 81, 98 and 108; 2, 14, 54, 81, 98 and 109; 1, 17, 54, 81, 98 and 107; 1, 17, 54, 81, 98 and 108; 1, 17, 54, 81, 98 and 109; 2, 17, 54, 81, 98 and 107; 2, 17, 54, 81, 98 and 108; 2, 17, 54, 81, 98 and 109; 1, 18, 54, 81, 98 and 107; 1, 18, 54, 81, 98 and 108; 1, 18, 54, 81, 98 and 109; 2, 18, 54, 81, 98 and 107; 2, 18, 54, 81, 98 and 108; 2, 18, 54, 81, 98 and 109; 1, 11, 55, 81, 98 and 107; 1, 11, 55, 81, 98 and 108; 1, 11, 55, 81, 98 and 109; 2, 11, 55, 81, 98 and 107; 2, 11, 55, 81, 98 and 108; 2, 11, 55, 81, 98 and 109; 1, 13, 55, 81, 98 and 107; 1, 13, 55, 81, 98 and 108; 1, 13, 55, 81, 98 and 109; 2, 13, 55, 81, 98 and 107; 2, 13, 55, 81, 98 and 108; 2, 13, 55, 81, 98 and 109; 1, 14, 55, 81, 98 and 107; 1, 14, 55, 81, 98 and 108; 1, 14, 55, 81, 98 and 109; 2, 14, 55, 81, 98 and 107; 2, 14, 55, 81, 98 and 108; 2, 14, 55, 81, 98 and 109; 1, 17, 55, 81, 98 and 107; 1, 17, 55, 81, 98 and 108; 1, 17, 55, 81, 98 and 109; 2, 17, 55, 81, 98 and 107; 2, 17, 55, 81, 98 and 108; 2, 17, 55, 81, 98 and 109; 1, 18, 55, 81, 98 and 107; 1, 18, 55, 81, 98 and 108; 1, 18, 55, 81, 98 and 109; 2, 18, 55, 81, 98 and 107; 2, 18, 55, 81, 98 and 108; 2, 18, 55, 81, 98 and 109; 1, 11, 53, 82, 98 and 107; 1, 11, 53, 82, 98 and 108; 1, 11, 53, 82, 98 and 109; 2, 11, 53, 82, 98 and 107; 2, 11, 53, 82, 98 and 108; 2, 11, 53, 82, 98 and 109; 1, 13, 53, 82, 98 and 107; 1, 13, 53, 82, 98 and 108; 1, 13, 53, 82, 98 and 109; 2, 13, 53, 82, 98 and 107; 2, 13, 53, 82, 98 and 108; 2, 13, 53, 82, 98 and 109; 1, 14, 53, 82, 98 and 107; 1, 14, 53, 82, 98 and 108; 1, 14, 53, 82, 98 and 109; 2, 14, 53, 82, 98 and 107; 2, 14, 53, 82, 98 and 108; 2, 14, 53, 82, 98 and 109; 1, 17, 53, 82, 98 and 107; 1, 17, 53, 82, 98 and 108; 1, 17, 53, 82, 98 and 109; 2, 17, 53, 82, 98 and 107; 2, 17, 53, 82, 98 and 108; 2, 17, 53, 82, 98 and 109; 1, 18, 53, 82, 98 and 107; 1, 18, 53, 82, 98 and 108; 1, 18, 53, 82, 98 and 109; 2, 18, 53, 82, 98 and 107; 2, 18, 53, 82, 98 and 108; 2, 18, 53, 82, 98 and 109; 1, 11, 54, 82, 98 and 107; 1, 11, 54, 82, 98 and 108; 1, 11, 54, 82, 98 and 109; 2, 11, 54, 82, 98 and 107; 2, 11, 54, 82, 98 and 108; 2, 11, 54, 82, 98 and 109; 1, 13, 54, 82, 98 and 107; 1, 13, 54, 82, 98 and 108; 1, 13, 54, 82, 98 and 109; 2, 13, 54, 82, 98 and 107; 2, 13, 54, 82, 98 and 108; 2, 13, 54, 82, 98 and 109; 1, 14, 54, 82, 98 and 107; 1, 14, 54, 82, 98 and 108; 1, 14, 54, 82, 98 and 109; 2, 14, 54, 82, 98 and 107; 2, 14, 54, 82, 98 and 108; 2, 14, 54, 82, 98 and 109; 1, 17, 54, 82, 98 and 107; 1, 17, 54, 82, 98 and 108; 1, 17, 54, 82, 98 and 109; 2, 17, 54, 82, 98 and 107; 2, 17, 54, 82, 98 and 108; 2, 17, 54, 82, 98 and 109; 1, 18, 54, 82, 98 and 107; 1, 18, 54, 82, 98 and 108; 1, 18, 54, 82, 98 and 109; 2, 18, 54, 82, 98 and 107; 2, 18, 54, 82, 98 and 108; 2, 18, 54, 82, 98 and 109; 1, 11, 55, 82, 98 and 107; 1, 11, 55, 82, 98 and 108; 1, 11, 55, 82, 98 and 109; 2, 11, 55, 82, 98 and 107; 2, 11, 55, 82, 98 and 108; 2, 11, 55, 82, 98 and 109; 1, 13, 55, 82, 98 and 107; 1, 13, 55, 82, 98 and 108; 1, 13, 55, 82, 98 and 109; 2, 13, 55, 82, 98 and 107; 2, 13, 55, 82, 98 and 108; 2, 13, 55, 82, 98 and 109; 1, 14, 55, 82, 98 and 107; 1, 14, 55, 82, 98 and 108; 1, 14, 55, 82, 98 and 109; 2, 14, 55, 82, 98 and 107; 2, 14, 55, 82, 98 and 108; 2, 14, 55, 82, 98 and 109; 1, 17, 55, 82, 98 and 107; 1, 17, 55, 82, 98 and 108; 1, 17, 55, 82, 98 and 109; 2, 17, 55, 82, 98 and 107; 2, 17, 55, 82, 98 and 108; 2, 17, 55, 82, 98 and 109; 1, 18, 55, 82, 98 and 107; 1, 18, 55, 82, 98 and 108; 1, 18, 55, 82, 98 and 109; 2, 18, 55, 82, 98 and 107; 2, 18, 55, 82, 98 and 108; or 2, 18, 55, 82, 98 and 109.

Also preferably, the vaccine provides combinations of at least six discrete immunization peptides comprising SEQ ID NO:108, more preferably provides immunization polypeptides consisting of amino acids of SEQ ID NOs:1, 11, 53, 80, 81 and 107; 2, 11, 53, 80, 81 and 107; 1, 13, 53, 80, 81 and 107; 2, 13, 53, 80, 81 and 107; 1, 14, 53, 80, 81 and 107; 2, 14, 53, 80, 81 and 107; 1, 17, 53, 80, 81 and 107; 2, 17, 53, 80, 81 and 107; 1, 18, 53, 80, 81 and 107; 2, 18, 53, 80, 81 and 107; 1, 11, 54, 80, 81 and 107; 2, 11, 54, 80, 81 and 107; 1, 13, 54, 80, 81 and 107; 2, 13, 54, 80, 81 and 107; 1, 14, 54, 80, 81 and 107; 2, 14, 54, 80, 81 and 107; 1, 17, 54, 80, 81 and 107; 2, 17, 54, 80, 81 and 107; 1, 18, 54, 80, 81 and 107; 2, 18, 54, 80, 81 and 107; 1, 11, 55, 80, 81 and 107; 2, 11, 55, 80, 81 and 107; 1, 13, 55, 80, 81 and 107; 2, 13, 55, 80, 81 and 107; 1, 14, 55, 80, 81 and 107; 2, 14, 55, 80, 81 and 107; 1, 17, 55, 80, 81 and 107; 2, 17, 55, 80, 81 and 107; 1, 18, 55, 80, 81 and 107; 2, 18, 55, 80, 81 and 107; 1, 11, 53, 82, 81 and 107; 2, 11, 53, 82, 81 and 107; 1, 13, 53, 82, 81 and 107; 2, 13, 53, 82, 81 and 107; 1, 14, 53, 82, 81 and 107; 2, 14, 53, 82, 81 and 107; 1, 17, 53, 82, 81 and 107; 2, 17, 53, 82, 81 and 107; 1, 18, 53, 82, 81 and 107; 2, 18, 53, 82, 81 and 107; 1, 11, 54, 82, 81 and 107; 2, 11, 54, 82, 81 and 107; 1, 13, 54, 82, 81 and 107; 2, 13, 54, 82, 81 and 107; 1, 14, 54, 82, 81 and 107; 2, 14, 54, 82, 81 and 107; 1, 17, 54, 82, 81 and 107; 2, 17, 54, 82, 81 and 107; 1, 18, 54, 82, 81 and 107; 2, 18, 54, 82, 81 and 107; 1, 11, 55, 82, 81 and 107; 2, 11, 55, 82, 81 and 107; 1, 13, 55, 82, 81 and 107; 2, 13, 55, 82, 81 and 107; 1, 14, 55, 82, 81 and 107; 2, 14, 55, 82, 81 and 107; 1, 17, 55, 82, 81 and 107; 2, 17, 55, 82, 81 and 107; 1, 18, 55, 82, 81 and 107; 2, 18, 55, 82, 81 and 107; 1, 11, 53, 80, 97 and 107; 2, 11, 53, 80, 97 and 107; 1, 13, 53, 80, 97 and 107; 2, 13, 53, 80, 97 and 107; 1, 14, 53, 80, 97 and 107; 2, 14, 53, 80, 97 and 107; 1, 17, 53, 80, 97 and 107; 2, 17, 53, 80, 97 and 107; 1, 18, 53, 80, 97 and 107; 2, 18, 53, 80, 97 and 107; 1, 11, 54, 80, 97 and 107; 2, 11, 54, 80, 97 and 107; 1, 13, 54, 80, 97 and 107; 2, 13, 54, 80, 97 and 107; 1, 14, 54, 80, 97 and 107; 2, 14, 54, 80, 97 and 107; 1, 17, 54, 80, 97 and 107; 2, 17, 54, 80, 97 and 107; 1, 18, 54, 80, 97 and 107; 2, 18, 54, 80, 97 and 107; 1, 11, 55, 80, 97 and 107; 2, 11, 55, 80, 97 and 107; 1, 13, 55, 80, 97 and 107; 2, 13, 55, 80, 97 and 107; 1, 14, 55, 80, 97 and 107; 2, 14, 55, 80, 97 and 107; 1, 17, 55, 80, 97 and 107; 2, 17, 55, 80, 97 and 107; 1, 18, 55, 80, 97 and 107; 2, 18, 55, 80, 97 and 107; 1, 11, 53, 81, 97 and 107; 2, 11, 53, 81, 97 and 107; 1, 13, 53, 81, 97 and 107; 2, 13, 53, 81, 97 and 107; 1, 14, 53, 81, 97 and 107; 2, 14, 53, 81, 97 and 107; 1, 17, 53, 81, 97 and 107; 2, 17, 53, 81, 97 and 107; 1, 18, 53, 81, 97 and 107; 2, 18, 53, 81, 97 and 107; 1, 11, 54, 81, 97 and 107; 2, 11, 54, 81, 97 and 107; 1, 13, 54, 81, 97 and 107; 2, 13, 54, 81, 97 and 107; 1, 14, 54, 81, 97 and 107; 2, 14, 54, 81, 97 and 107; 1, 17, 54, 81, 97 and 107; 2, 17, 54, 81, 97 and 107; 1, 18, 54, 81, 97 and 107; 2, 18, 54, 81, 97 and 107; 1, 11, 55, 81, 97 and 107; 2, 11, 55, 81, 97 and 107; 1, 13, 55, 81, 97 and 107; 2, 13, 55, 81, 97 and 107; 1, 14, 55, 81, 97 and 107; 2, 14, 55, 81, 97 and 107; 1, 17, 55, 81, 97 and 107; 2, 17, 55, 81, 97 and 107; 1, 18, 55, 81, 97 and 107; 2, 18, 55, 81, 97 and 107; 1, 11, 53, 82, 97 and 107; 2, 11, 53, 82, 97 and 107; 1, 13, 53, 82, 97 and 107; 2, 13, 53, 82, 97 and 107; 1, 14, 53, 82, 97 and 107; 2, 14, 53, 82, 97 and 107; 1, 17, 53, 82, 97 and 107; 2, 17, 53, 82, 97 and 107; 1, 18, 53, 82, 97 and 107; 2, 18, 53, 82, 97 and 107; 1, 11, 54, 82, 97 and 107; 2, 11, 54, 82, 97 and 107; 1, 13, 54, 82, 97 and 107; 2, 13, 54, 82, 97 and 107; 1, 14, 54, 82, 97 and 107; 2, 14, 54, 82, 97 and 107; 1, 17, 54, 82, 97 and 107; 2, 17, 54, 82, 97 and 107; 1, 18, 54, 82, 97 and 107; 2, 18, 54, 82, 97 and 107; 1, 11, 55, 82, 97 and 107; 2, 11, 55, 82, 97 and 107; 1, 13, 55, 82, 97 and 107; 2, 13, 55, 82, 97 and 107; 1, 14, 55, 82, 97 and 107; 2, 14, 55, 82, 97 and 107; 1, 17, 55, 82, 97 and 107; 2, 17, 55, 82, 97 and 107; 1, 18, 55, 82, 97 and 107; 2, 18, 55, 82, 97 and 107; 1, 11, 53, 80, 98 and 107; 2, 11, 53, 80, 98 and 107; 1, 13, 53, 80, 98 and 107; 2, 13, 53, 80, 98 and 107; 1, 14, 53, 80, 98 and 107; 2, 14, 53, 80, 98 and 107; 1, 17, 53, 80, 98 and 107; 2, 17, 53, 80, 98 and 107; 1, 18, 53, 80, 98 and 107; 2, 18, 53, 80, 98 and 107; 1, 11, 54, 80, 98 and 107; 2, 11, 54, 80, 98 and 107; 1, 13, 54, 80, 98 and 107; 2, 13, 54, 80, 98 and 107; 1, 14, 54, 80, 98 and 107; 2, 14, 54, 80, 98 and 107; 1, 17, 54, 80, 98 and 107; 2, 17, 54, 80, 98 and 107; 1, 18, 54, 80, 98 and 107; 2, 18, 54, 80, 98 and 107; 1, 11, 55, 80, 98 and 107; 2, 11, 55, 80, 98 and 107; 1, 13, 55, 80, 98 and 107; 2, 13, 55, 80, 98 and 107; 1, 14, 55, 80, 98 and 107; 2, 14, 55, 80, 98 and 107; 1, 17, 55, 80, 98 and 107; 2, 17, 55, 80, 98 and 107; 1, 18, 55, 80, 98 and 107; 2, 18, 55, 80, 98 and 107; 1, 11, 53, 81, 98 and 107; 2, 11, 53, 81, 98 and 107; 1, 13, 53, 81, 98 and 107; 2, 13, 53, 81, 98 and 107; 1, 14, 53, 81, 98 and 107; 2, 14, 53, 81, 98 and 107; 1, 17, 53, 81, 98 and 107; 2, 17, 53, 81, 98 and 107; 1, 18, 53, 81, 98 and 107; 2, 18, 53, 81, 98 and 107; 1, 11, 54, 81, 98 and 107; 2, 11, 54, 81, 98 and 107; 1, 13, 54, 81, 98 and 107; 2, 13, 54, 81, 98 and 107; 1, 14, 54, 81, 98 and 107; 2, 14, 54, 81, 98 and 107; 1, 17, 54, 81, 98 and 107; 2, 17, 54, 81, 98 and 107; 1, 18, 54, 81, 98 and 107; 2, 18, 54, 81, 98 and 107; 1, 11, 55, 81, 98 and 107; 2, 11, 55, 81, 98 and 107; 1, 13, 55, 81, 98 and 107; 2, 13, 55, 81, 98 and 107; 1, 14, 55, 81, 98 and 107; 2, 14, 55, 81, 98 and 107; 1, 17, 55, 81, 98 and 107; 2, 17, 55, 81, 98 and 107; 1, 18, 55, 81, 98 and 107; 2, 18, 55, 81, 98 and 107; 1, 11, 53, 82, 98 and 107; 2, 11, 53, 82, 98 and 107; 1, 13, 53, 82, 98 and 107; 2, 13, 53, 82, 98 and 107; 1, 14, 53, 82, 98 and 107; 2, 14, 53, 82, 98 and 107; 1, 17, 53, 82, 98 and 107; 2, 17, 53, 82, 98 and 107; 1, 18, 53, 82, 98 and 107; 2, 18, 53, 82, 98 and 107; 1, 11, 54, 82, 98 and 107; 2, 11, 54, 82, 98 and 107; 1, 13, 54, 82, 98 and 107; 2, 13, 54, 82, 98 and 107; 1, 14, 54, 82, 98 and 107; 2, 14, 54, 82, 98 and 107; 1, 17, 54, 82, 98 and 107; 2, 17, 54, 82, 98 and 107; 1, 18, 54, 82, 98 and 107; 2, 18, 54, 82, 98 and 107; 1, 11, 55, 82, 98 and 107; 2, 11, 55, 82, 98 and 107; 1, 13, 55, 82, 98 and 107; 2, 13, 55, 82, 98 and 107; 1, 14, 55, 82, 98 and 107; 2, 14, 55, 82, 98 and 107; 1, 17, 55, 82, 98 and 107; 2, 17, 55, 82, 98 and 107; 1, 18, 55, 82, 98 and 107; or 2, 18, 55, 82, 98 and 107.

Also preferably, the vaccine provides combinations of at least six discrete immunization peptides comprising SEQ ID NO:108, more preferably provides immunization polypeptides consisting of amino acids of SEQ ID NOs:1, 11, 53, 80, 81 and 108; 2, 11, 53, 80, 81 and 108; 1, 13, 53, 80, 81 and 108; 2, 13, 53, 80, 81 and 108; 1, 14, 53, 80, 81 and 108; 2, 14, 53, 80, 81 and 108; 1, 17, 53, 80, 81 and 108; 2, 17, 53, 80, 81 and 108; 1, 18, 53, 80, 81 and 108; 2, 18, 53, 80, 81 and 108; 1, 11, 54, 80, 81 and 108; 2, 11, 54, 80, 81 and 108; 1, 13, 54, 80, 81 and 108; 2, 13, 54, 80, 81 and 108; 1, 14, 54, 80, 81 and 108; 2, 14, 54, 80, 81 and 108; 1, 17, 54, 80, 81 and 108; 2, 17, 54, 80, 81 and 108; 1, 18, 54, 80, 81 and 108; 2, 18, 54, 80, 81 and 108; 1, 11, 55, 80, 81 and 108; 2, 11, 55, 80, 81 and 108; 1, 13, 55, 80, 81 and 108; 2, 13, 55, 80, 81 and 108; 1, 14, 55, 80, 81 and 108; 2, 14, 55, 80, 81 and 108; 1, 17, 55, 80, 81 and 108; 2, 17, 55, 80, 81 and 108; 1, 18, 55, 80, 81 and 108; 2, 18, 55, 80, 81 and 108; 1, 11, 53, 82, 81 and 108; 2, 11, 53, 82, 81 and 108; 1, 13, 53, 82, 81 and 108; 2, 13, 53, 82, 81 and 108; 1, 14, 53, 82, 81 and 108; 2, 14, 53, 82, 81 and 108; 1, 17, 53, 82, 81 and 108; 2, 17, 53, 82, 81 and 108; 1, 18, 53, 82, 81 and 108; 2, 18, 53, 82, 81 and 108; 1, 11, 54, 82, 81 and 108; 2, 11, 54, 82, 81 and 108; 1, 13, 54, 82, 81 and 108; 2, 13, 54, 82, 81 and 108; 1, 14, 54, 82, 81 and 108; 2, 14, 54, 82, 81 and 108; 1, 17, 54, 82, 81 and 108; 2, 17, 54, 82, 81 and 108; 1, 18, 54, 82, 81 and 108; 2, 18, 54, 82, 81 and 108; 1, 11, 55, 82, 81 and 108; 2, 11, 55, 82, 81 and 108; 1, 13, 55, 82, 81 and 108; 2, 13, 55, 82, 81 and 108; 1, 14, 55, 82, 81 and 108; 2, 14, 55, 82, 81 and 108; 1, 17, 55, 82, 81 and 108; 2, 17, 55, 82, 81 and 108; 1, 18, 55, 82, 81 and 108; 2, 18, 55, 82, 81 and 108; 1, 11, 53, 80, 97 and 108; 2, 11, 53, 80, 97 and 108; 1, 13, 53, 80, 97 and 108; 2, 13, 53, 80, 97 and 108; 1, 14, 53, 80, 97 and 108; 2, 14, 53, 80, 97 and 108; 1, 17, 53, 80, 97 and 108; 2, 17, 53, 80, 97 and 108; 1, 18, 53, 80, 97 and 108; 2, 18, 53, 80, 97 and 108; 1, 11, 54, 80, 97 and 108; 2, 11, 54, 80, 97 and 108; 1, 13, 54, 80, 97 and 108; 2, 13, 54, 80, 97 and 108; 1, 14, 54, 80, 97 and 108; 2, 14, 54, 80, 97 and 108; 1, 17, 54, 80, 97 and 108; 2, 17, 54, 80, 97 and 108; 1, 18, 54, 80, 97 and 108; 2, 18, 54, 80, 97 and 108; 1, 11, 55, 80, 97 and 108; 2, 11, 55, 80, 97 and 108; 1, 13, 55, 80, 97 and 108; 2, 13, 55, 80, 97 and 108; 1, 14, 55, 80, 97 and 108; 2, 14, 55, 80, 97 and 108; 1, 17, 55, 80, 97 and 108; 2, 17, 55, 80, 97 and 108; 1, 18, 55, 80, 97 and 108; 2, 18, 55, 80, 97 and 108; 1, 11, 53, 81, 97 and 108; 2, 11, 53, 81, 97 and 108; 1, 13, 53, 81, 97 and 108; 2, 13, 53, 81, 97 and 108; 1, 14, 53, 81, 97 and 108; 2, 14, 53, 81, 97 and 108; 1, 17, 53, 81, 97 and 108; 2, 17, 53, 81, 97 and 108; 1, 18, 53, 81, 97 and 108; 2, 18, 53, 81, 97 and 108; 1, 11, 54, 81, 97 and 108; 2, 11, 54, 81, 97 and 108; 1, 13, 54, 81, 97 and 108; 2, 13, 54, 81, 97 and 108; 1, 14, 54, 81, 97 and 108; 2, 14, 54, 81, 97 and 108; 1, 17, 54, 81, 97 and 108; 2, 17, 54, 81, 97 and 108; 1, 18, 54, 81, 97 and 108; 2, 18, 54, 81, 97 and 108; 1, 11, 55, 81, 97 and 108; 2, 11, 55, 81, 97 and 108; 1, 13, 55, 81, 97 and 108; 2, 13, 55, 81, 97 and 108; 1, 14, 55, 81, 97 and 108; 2, 14, 55, 81, 97 and 108; 1, 17, 55, 81, 97 and 108; 2, 17, 55, 81, 97 and 108; 1, 18, 55, 81, 97 and 108; 2, 18, 55, 81, 97 and 108; 1, 11, 53, 82, 97 and 108; 2, 11, 53, 82, 97 and 108; 1, 13, 53, 82, 97 and 108; 2, 13, 53, 82, 97 and 108; 1, 14, 53, 82, 97 and 108; 2, 14, 53, 82, 97 and 108; 1, 17, 53, 82, 97 and 108; 2, 17, 53, 82, 97 and 108; 1, 18, 53, 82, 97 and 108; 2, 18, 53, 82, 97 and 108; 1, 11, 54, 82, 97 and 108; 2, 11, 54, 82, 97 and 108; 1, 13, 54, 82, 97 and 108; 2, 13, 54, 82, 97 and 108; 1, 14, 54, 82, 97 and 108; 2, 14, 54, 82, 97 and 108; 1, 17, 54, 82, 97 and 108; 2, 17, 54, 82, 97 and 108; 1, 18, 54, 82, 97 and 108; 2, 18, 54, 82, 97 and 108; 1, 11, 55, 82, 97 and 108; 2, 11, 55, 82, 97 and 108; 1, 13, 55, 82, 97 and 108; 2, 13, 55, 82, 97 and 108; 1, 14, 55, 82, 97 and 108; 2, 14, 55, 82, 97 and 108; 1, 17, 55, 82, 97 and 108; 2, 17, 55, 82, 97 and 108; 1, 18, 55, 82, 97 and 108; 2, 18, 55, 82, 97 and 108; 1, 11, 53, 80, 98 and 108; 2, 11, 53, 80, 98 and 108; 1, 13, 53, 80, 98 and 108; 2, 13, 53, 80, 98 and 108; 1, 14, 53, 80, 98 and 108; 2, 14, 53, 80, 98 and 108; 1, 17, 53, 80, 98 and 108; 2, 17, 53, 80, 98 and 108; 1, 18, 53, 80, 98 and 108; 2, 18, 53, 80, 98 and 108; 1, 11, 54, 80, 98 and 108; 2, 11, 54, 80, 98 and 108; 1, 13, 54, 80, 98 and 108; 2, 13, 54, 80, 98 and 108; 1, 14, 54, 80, 98 and 108; 2, 14, 54, 80, 98 and 108; 1, 17, 54, 80, 98 and 108; 2, 17, 54, 80, 98 and 108; 1, 18, 54, 80, 98 and 108; 2, 18, 54, 80, 98 and 108; 1, 11, 55, 80, 98 and 108; 2, 11, 55, 80, 98 and 108; 1, 13, 55, 80, 98 and 108; 2, 13, 55, 80, 98 and 108; 1, 14, 55, 80, 98 and 108; 2, 14, 55, 80, 98 and 108; 1, 17, 55, 80, 98 and 108; 2, 17, 55, 80, 98 and 108; 1, 18, 55, 80, 98 and 108; 2, 18, 55, 80, 98 and 108; 1, 11, 53, 81, 98 and 108; 2, 11, 53, 81, 98 and 108; 1, 13, 53, 81, 98 and 108; 2, 13, 53, 81, 98 and 108; 1, 14, 53, 81, 98 and 108; 2, 14, 53, 81, 98 and 108; 1, 17, 53, 81, 98 and 108; 2, 17, 53, 81, 98 and 108; 1, 18, 53, 81, 98 and 108; 2, 18, 53, 81, 98 and 108; 1, 11, 54, 81, 98 and 108; 2, 11, 54, 81, 98 and 108; 1, 13, 54, 81, 98 and 108; 2, 13, 54, 81, 98 and 108; 1, 14, 54, 81, 98 and 108; 2, 14, 54, 81, 98 and 108; 1, 17, 54, 81, 98 and 108; 2, 17, 54, 81, 98 and 108; 1, 18, 54, 81, 98 and 108; 2, 18, 54, 81, 98 and 108; 1, 11, 55, 81, 98 and 108; 2, 11, 55, 81, 98 and 108; 1, 13, 55, 81, 98 and 108; 2, 13, 55, 81, 98 and 108; 1, 14, 55, 81, 98 and 108; 2, 14, 55, 81, 98 and 108; 1, 17, 55, 81, 98 and 108; 2, 17, 55, 81, 98 and 108; 1, 18, 55, 81, 98 and 108; 2, 18, 55, 81, 98 and 108; 1, 11, 53, 82, 98 and 108; 2, 11, 53, 82, 98 and 108; 1, 13, 53, 82, 98 and 108; 2, 13, 53, 82, 98 and 108; 1, 14, 53, 82, 98 and 108; 2, 14, 53, 82, 98 and 108; 1, 17, 53, 82, 98 and 108; 2, 17, 53, 82, 98 and 108; 1, 18, 53, 82, 98 and 108; 2, 18, 53, 82, 98 and 108; 1, 11, 54, 82, 98 and 108; 2, 11, 54, 82, 98 and 108; 1, 13, 54, 82, 98 and 108; 2, 13, 54, 82, 98 and 108; 1, 14, 54, 82, 98 and 108; 2, 14, 54, 82, 98 and 108; 1, 17, 54, 82, 98 and 108; 2, 17, 54, 82, 98 and 108; 1, 18, 54, 82, 98 and 108; 2, 18, 54, 82, 98 and 108; 1, 11, 55, 82, 98 and 108; 2, 11, 55, 82, 98 and 108; 1, 13, 55, 82, 98 and 108; 2, 13, 55, 82, 98 and 108; 1, 14, 55, 82, 98 and 108; 2, 14, 55, 82, 98 and 108; 1, 17, 55, 82, 98 and 108; 2, 17, 55, 82, 98 and 108; 1, 18, 55, 82, 98 and 108; or 2, 18, 55, 82, 98 and 108.

Also preferably, the vaccine provides combinations of at least six discrete immunization peptides comprising SEQ ID NO:109, more preferably provides immunization polypeptides consisting of amino acids of SEQ ID NOs:1, 11, 53, 80, 81 and 109; 2, 11, 53, 80, 81 and 109; 1, 13, 53, 80, 81 and 109; 2, 13, 53, 80, 81 and 109; 1, 14, 53, 80, 81 and 109; 2, 14, 53, 80, 81 and 109; 1, 17, 53, 80, 81 and 109; 2, 17, 53, 80, 81 and 109; 1, 18, 53, 80, 81 and 109; 2, 18, 53, 80, 81 and 109; 1, 11, 54, 80, 81 and 109; 2, 11, 54, 80, 81 and 109; 1, 13, 54, 80, 81 and 109; 2, 13, 54, 80, 81 and 109; 1, 14, 54, 80, 81 and 109; 2, 14, 54, 80, 81 and 109; 1, 17, 54, 80, 81 and 109; 2, 17, 54, 80, 81 and 109; 1, 18, 54, 80, 81 and 109; 2, 18, 54, 80, 81 and 109; 1, 11, 55, 80, 81 and 109; 2, 11, 55, 80, 81 and 109; 1, 13, 55, 80, 81 and 109; 2, 13, 55, 80, 81 and 109; 1, 14, 55, 80, 81 and 109; 2, 14, 55, 80, 81 and 109; 1, 17, 55, 80, 81 and 109; 2, 17, 55, 80, 81 and 109; 1, 18, 55, 80, 81 and 109; 2, 18, 55, 80, 81 and 109; 1, 11, 53, 82, 81 and 109; 2, 11, 53, 82, 81 and 109; 1, 13, 53, 82, 81 and 109; 2, 13, 53, 82, 81 and 109; 1, 14, 53, 82, 81 and 109; 2, 14, 53, 82, 81 and 109; 1, 17, 53, 82, 81 and 109; 2, 17, 53, 82, 81 and 109; 1, 18, 53, 82, 81 and 109; 2, 18, 53, 82, 81 and 109; 1, 11, 54, 82, 81 and 109; 2, 11, 54, 82, 81 and 109; 1, 13, 54, 82, 81 and 109; 2, 13, 54, 82, 81 and 109; 1, 14, 54, 82, 81 and 109; 2, 14, 54, 82, 81 and 109; 1, 17, 54, 82, 81 and 109; 2, 17, 54, 82, 81 and 109; 1, 18, 54, 82, 81 and 109; 2, 18, 54, 82, 81 and 109; 1, 11, 55, 82, 81 and 109; 2, 11, 55, 82, 81 and 109; 1, 13, 55, 82, 81 and 109; 2, 13, 55, 82, 81 and 109; 1, 14, 55, 82, 81 and 109; 2, 14, 55, 82, 81 and 109; 1, 17, 55, 82, 81 and 109; 2, 17, 55, 82, 81 and 109; 1, 18, 55, 82, 81 and 109; 2, 18, 55, 82, 81 and 109; 1, 11, 53, 80, 97 and 109; 2, 11, 53, 80, 97 and 109; 1, 13, 53, 80, 97 and 109; 2, 13, 53, 80, 97 and 109; 1, 14, 53, 80, 97 and 109; 2, 14, 53, 80, 97 and 109; 1, 17, 53, 80, 97 and 109; 2, 17, 53, 80, 97 and 109; 1, 18, 53, 80, 97 and 109; 2, 18, 53, 80, 97 and 109; 1, 11, 54, 80, 97 and 109; 2, 11, 54, 80, 97 and 109; 1, 13, 54, 80, 97 and 109; 2, 13, 54, 80, 97 and 109; 1, 14, 54, 80, 97 and 109; 2, 14, 54, 80, 97 and 109; 1, 17, 54, 80, 97 and 109; 2, 17, 54, 80, 97 and 109; 1, 18, 54, 80, 97 and 109; 2, 18, 54, 80, 97 and 109; 1, 11, 55, 80, 97 and 109; 2, 11, 55, 80, 97 and 109; 1, 13, 55, 80, 97 and 109; 2, 13, 55, 80, 97 and 109; 1, 14, 55, 80, 97 and 109; 2, 14, 55, 80, 97 and 109; 1, 17, 55, 80, 97 and 109; 2, 17, 55, 80, 97 and 109; 1, 18, 55, 80, 97 and 109; 2, 18, 55, 80, 97 and 109; 1, 11, 53, 81, 97 and 109; 2, 11, 53, 81, 97 and 109; 1, 13, 53, 81, 97 and 109; 2, 13, 53, 81, 97 and 109; 1, 14, 53, 81, 97 and 109; 2, 14, 53, 81, 97 and 109; 1, 17, 53, 81, 97 and 109; 2, 17, 53, 81, 97 and 109; 1, 18, 53, 81, 97 and 109; 2, 18, 53, 81, 97 and 109; 1, 11, 54, 81, 97 and 109; 2, 11, 54, 81, 97 and 109; 1, 13, 54, 81, 97 and 109; 2, 13, 54, 81, 97 and 109; 1, 14, 54, 81, 97 and 109; 2, 14, 54, 81, 97 and 109; 1, 17, 54, 81, 97 and 109; 2, 17, 54, 81, 97 and 109; 1, 18, 54, 81, 97 and 109; 2, 18, 54, 81, 97 and 109; 1, 11, 55, 81, 97 and 109; 2, 11, 55, 81, 97 and 109; 1, 13, 55, 81, 97 and 109; 2, 13, 55, 81, 97 and 109; 1, 14, 55, 81, 97 and 109; 2, 14, 55, 81, 97 and 109; 1, 17, 55, 81, 97 and 109; 2, 17, 55, 81, 97 and 109; 1, 18, 55, 81, 97 and 109; 2, 18, 55, 81, 97 and 109; 1, 11, 53, 82, 97 and 109; 2, 11, 53, 82, 97 and 109; 1, 13, 53, 82, 97 and 109; 2, 13, 53, 82, 97 and 109; 1, 14, 53, 82, 97 and 109; 2, 14, 53, 82, 97 and 109; 1, 17, 53, 82, 97 and 109; 2, 17, 53, 82, 97 and 109; 1, 18, 53, 82, 97 and 109; 2, 18, 53, 82, 97 and 109; 1, 11, 54, 82, 97 and 109; 2, 11, 54, 82, 97 and 109; 1, 13, 54, 82, 97 and 109; 2, 13, 54, 82, 97 and 109; 1, 14, 54, 82, 97 and 109; 2, 14, 54, 82, 97 and 109; 1, 17, 54, 82, 97 and 109; 2, 17, 54, 82, 97 and 109; 1, 18, 54, 82, 97 and 109; 2, 18, 54, 82, 97 and 109; 1, 11, 55, 82, 97 and 109; 2, 11, 55, 82, 97 and 109; 1, 13, 55, 82, 97 and 109; 2, 13, 55, 82, 97 and 109; 1, 14, 55, 82, 97 and 109; 2, 14, 55, 82, 97 and 109; 1, 17, 55, 82, 97 and 109; 2, 17, 55, 82, 97 and 109; 1, 18, 55, 82, 97 and 109; 2, 18, 55, 82, 97 and 109; 1, 11, 53, 80, 98 and 109; 2, 11, 53, 80, 98 and 109; 1, 13, 53, 80, 98 and 109; 2, 13, 53, 80, 98 and 109; 1, 14, 53, 80, 98 and 109; 2, 14, 53, 80, 98 and 109; 1, 17, 53, 80, 98 and 109; 2, 17, 53, 80, 98 and 109; 1, 18, 53, 80, 98 and 109; 2, 18, 53, 80, 98 and 109; 1, 11, 54, 80, 98 and 109; 2, 11, 54, 80, 98 and 109; 1, 13, 54, 80, 98 and 109; 2, 13, 54, 80, 98 and 109; 1, 14, 54, 80, 98 and 109; 2, 14, 54, 80, 98 and 109; 1, 17, 54, 80, 98 and 109; 2, 17, 54, 80, 98 and 109; 1, 18, 54, 80, 98 and 109; 2, 18, 54, 80, 98 and 109; 1, 11, 55, 80, 98 and 109; 2, 11, 55, 80, 98 and 109; 1, 13, 55, 80, 98 and 109; 2, 13, 55, 80, 98 and 109; 1, 14, 55, 80, 98 and 109; 2, 14, 55, 80, 98 and 109; 1, 17, 55, 80, 98 and 109; 2, 17, 55, 80, 98 and 109; 1, 18, 55, 80, 98 and 109; 2, 18, 55, 80, 98 and 109; 1, 11, 53, 81, 98 and 109; 2, 11, 53, 81, 98 and 109; 1, 13, 53, 81, 98 and 109; 2, 13, 53, 81, 98 and 109; 1, 14, 53, 81, 98 and 109; 2, 14, 53, 81, 98 and 109; 1, 17, 53, 81, 98 and 109; 2, 17, 53, 81, 98 and 109; 1, 18, 53, 81, 98 and 109; 2, 18, 53, 81, 98 and 109; 1, 11, 54, 81, 98 and 109; 2, 11, 54, 81, 98 and 109; 1, 13, 54, 81, 98 and 109; 2, 13, 54, 81, 98 and 109; 1, 14, 54, 81, 98 and 109; 2, 14, 54, 81, 98 and 109; 1, 17, 54, 81, 98 and 109; 2, 17, 54, 81, 98 and 109; 1, 18, 54, 81, 98 and 109; 2, 18, 54, 81, 98 and 109; 1, 11, 55, 81, 98 and 109; 2, 11, 55, 81, 98 and 109; 1, 13, 55, 81, 98 and 109; 2, 13, 55, 81, 98 and 109; 1, 14, 55, 81, 98 and 109; 2, 14, 55, 81, 98 and 109; 1, 17, 55, 81, 98 and 109; 2, 17, 55, 81, 98 and 109; 1, 18, 55, 81, 98 and 109; 2, 18, 55, 81, 98 and 109; 1, 11, 53, 82, 98 and 109; 2, 11, 53, 82, 98 and 109; 1, 13, 53, 82, 98 and 109; 2, 13, 53, 82, 98 and 109; 1, 14, 53, 82, 98 and 109; 2, 14, 53, 82, 98 and 109; 1, 17, 53, 82, 98 and 109; 2, 17, 53, 82, 98 and 109; 1, 18, 53, 82, 98 and 109; 2, 18, 53, 82, 98 and 109; 1, 11, 54, 82, 98 and 109; 2, 11, 54, 82, 98 and 109; 1, 13, 54, 82, 98 and 109; 2, 13, 54, 82, 98 and 109; 1, 14, 54, 82, 98 and 109; 2, 14, 54, 82, 98 and 109; 1, 17, 54, 82, 98 and 109; 2, 17, 54, 82, 98 and 109; 1, 18, 54, 82, 98 and 109; 2, 18, 54, 82, 98 and 109; 1, 11, 55, 82, 98 and 109; 2, 11, 55, 82, 98 and 109; 1, 13, 55, 82, 98 and 109; 2, 13, 55, 82, 98 and 109; 1, 14, 55, 82, 98 and 109; 2, 14, 55, 82, 98 and 109; 1, 17, 55, 82, 98 and 109; 2, 17, 55, 82, 98 and 109; 1, 18, 55, 82, 98 and 109; or 2, 18, 55, 82, 98 and 109.

The present invention also relates to a method of inducing an immune response to a HPV16-related virus in a subject, comprising contacting said subject with a vaccine and/or a vector of the present invention.

The present invention further relates to a use of a vaccine and/or a vector in the manufacture of a medicament, preferably a vaccine.

The present invention moreover relates to a kit comprising the vaccine or vector of the present invention comprised in a housing.

The term “kit”, as used herein, refers to a collection of the aforementioned compounds, means or reagents. The components of the kit may be comprised by separate vials (i.e. as a kit of separate parts) or provided in a single vial, e.g. as a composition as specified herein above. The housing of the kit preferably allows translocation of the compounds of the kit, in particular common translocation; thus, the housing may in particular be a transportable container comprising all specified components. Moreover, it is to be understood that the kit of the present invention may be used for practicing at least one of the methods referred to herein above. It is envisaged that all components may be provided in a ready-to-use manner for practicing a method referred to above. Further, the kit preferably contains instructions for carrying out said methods, e.g. dosage instructions. The instructions can be provided by a user manual in paper- or electronic form. The kit preferably comprises further components, preferably at least one further therapeutic agent as specified herein above, an excipient, and/or a means of administration, in particular a syringe and/or a needle, and/or an IV infusion equipment.

Also, the present invention relates to a device comprising the vaccine and/or vector of the present invention.

The term “device”, as used herein relates to a system of means comprising at least the means operatively linked to each other as to allow administration of the vaccine and/or the vector. Preferred means for administering polynucleotides, compositions, or viruses are well known in the art. How to link the means in an operating manner will depend on the type of means included into the device and on the kind of administration envisaged. Preferably, the means are comprised by a single device in such a case. Said device may accordingly include a delivery unit for the administration of the compound or composition and a storage unit for storing said compound or composition until administration. However, it is also contemplated that the means of the current invention may appear as separate devices in such an embodiment and are, preferably, packaged together as a kit. The person skilled in the art will realize how to link the means without further ado. Preferred devices are those which can be applied without the particular knowledge of a specialized technician. Preferably, the device is a syringe, more preferably with a needle, comprising the compound or composition of the invention. Also preferably, the device is an intravenous infusion (IV) equipment comprising the compound or composition. Also preferably, the device is an endoscopic device comprising the compound or medicament for flushing a site of administration, or further comprising a needle for topical application of the compound or composition, e.g. to a tumor. Also preferably, the device is an inhaler comprising the compound of the present invention, wherein, more preferably, said compound is formulated for administration as an aerosol. Further preferably, the device is a cervical cap, a swab, a tampon, a vaginal ring, a vaginal strip, a vaginal capsule, a bioadhesive film, a sponge or a brush; in such case, the vaccine and/or vector may in particular be formulated as a cream, emulsion, nanoemulsion, or the like.

The present invention also relates to a method for stimulating T cells recognizing HPV16-related virus-infected host cells (anti-HPV16 T cells), said method comprising
(I) contacting an agent expressing MHC class I molecules with a vaccine or vector of as specified herein above, thereby causing presentation of at least one of said immunization peptides on said MHC class I molecules,
(II) contacting the agent expressing MHC class I molecules of step (i) with T cells, and, thereby, stimulating anti-HPV16 T cells.

The present invention also relates to a method of producing T cells recognizing HPV16-infected host cells (anti-HPV16 T cells), comprising (i) contacting a subject with a vaccine or vector as specified herein above and (ii) obtaining anti-HPV16 T cells. The present invention also relates to an anti-HPV T cell produced according to the aforesaid method for use in treating and/or preventing HPV16-related disease.

The present invention also relates to a method for treating a HPV16-positive inappropriate cellular proliferation in a subject, comprising
(I) administering a vaccine and/or vector as specified herein above to said subject; and
(II) thereby treating and/or preventing a HPV16-positive inappropriate cellular proliferation.

In view of the above, the following embodiments are particularly envisaged:

Embodiment 1: A vaccine providing at least one discrete immunization peptide consisting of an amino acid sequence selected from at least one of the following groups:
(i) SEQ ID NOs: 1 to 10;
(ii) SEQ ID NOs:11 to 52;
(iii) SEQ ID NOs:53 to 79;
(iv) SEQ ID NOs: 71 and 80 to 96;
(v) SEQ ID NOs:25, 81, and 97-106; and
(vi) SEQ ID NOs: 9, 12, 23, 63, 81, 85, 101, and 107 to 131.

Embodiment 2: The vaccine of embodiment 1, wherein said at least one immunization peptide has been verified to activate human anti-immunization peptide T cells when presented as a human lymphocyte antigen (HLA)-complex.

Embodiment 3: The vaccine of embodiment 1 or 2, wherein a HPV16-derived peptide consisting of the same amino acid sequence as the at least one immunization peptide has been verified to be presented by human HPV16-positive infected or tumor cells and wherein said at least one immunization peptide is selected from at least one of the following groups:
(i) SEQ ID NOs: 1 to 2;
(ii) SEQ ID NOs:11 to 23;
(iii) SEQ ID NOs:53 to 57;
(iv) SEQ ID NOs: 80 to 81;
(v) SEQ ID NOs: 81, 97, and 98; and
(vi) SEQ ID NO: 81, and 107-114.

Embodiment 4: The vaccine of any one of embodiments 1 to 3, wherein providing an immunization peptide is causing an immunization peptide to become presented as an HLA complex on the surface of an antigen presenting cell of the human subject after contacting the human subject with said vaccine.

Embodiment 5: The vaccine of any one of embodiments 1 to 4, wherein said providing is causing an immunization peptide to become present by processes occurring naturally in a host cell, in particular proteasome proteolysis and/or MHC class I presentation pathways.

Embodiment 6: The vaccine of any one of embodiments 1 to 5, providing at least four discrete immunization peptides consisting of amino acid sequences selected from at least four of said groups.

Embodiment 7: The vaccine of any one of embodiments 1 to 6, wherein said at least one immunization peptide is linked in said vaccine to at least one of a mediator of HLA presentation, a linker, a solubility mediator, a cell-penetrating peptide, a detectable tag, a mediator of secretion, and/or an immune stimulant, or is an amphiphilic conjugate thereof.

Embodiment 8: The vaccine of embodiment 6 or 7, wherein said vaccine comprises a mixture of discrete peptides each providing exactly one of said immunization peptides and/or comprises at least one polynucleotide providing said immunization peptides in a discrete manner.

Embodiment 9: The vaccine of any one of embodiments 6 to 8, wherein said vaccine is a universal vaccine providing immunization peptides such that at least 90%, preferably at least 95%, even more preferably at least 98%, most preferably at least 99%, of subjects of the world's population express at least one HLA of a HLA supertype capable of presenting at least one of said immunization peptides.

Embodiment 10: The vaccine of any one of embodiments 6 to 9, comprising at least one fusion polypeptide, wherein said fusion polypeptide comprises at least two of said immunization peptides.

Embodiment 11: The vaccine of any one of embodiments 6 to 10, comprising at least one fusion polypeptide, wherein said fusion polypeptide comprises all of said immunization peptides.

Embodiment 12: The vaccine of any one of embodiments 10 or 11, wherein any HPV16-derived amino acid sequence comprised in said fusion polypeptide consists of at most 12 contiguous HPV16-derived amino acids.

Embodiment 13: The vaccine of any one of embodiments 10 to 12, wherein each two of said immunization peptides in said fusion polypeptide are intervened by a non-HPV-derived amino acid sequence.

Embodiment 14: The vaccine of any one of embodiments 1 to 13, comprising at least one polynucleotide encoding at least one of said immunization peptide(s) and/or fusion polypeptide(s) of any one of embodiments 1 to 13, is a nanoemulsion comprising said immunization peptide(s) and/or fusion polypeptide(s), or is an antigen presenting cell (APC) or artificial APC presenting said peptide(s) and/or fusion polypeptide(s).

Embodiment 15: The vaccine of embodiment 14, wherein said polynucleotide is an RNA.

Embodiment 16: A vector comprising the HPV16 vaccine according to any one of embodiments 1 to 15.

Embodiment 17: A vaccine according to any one of embodiments 1 to 15 or a vector according to embodiment 16 for use in medicine.

Embodiment 18: A vaccine according to any one of embodiments 1 to 15 or a vector according to embodiment 16 for use in eliciting an immune response in a human subject against an HPV16-related infection.

Embodiment 19: The vaccine or vector for use of embodiment 18, wherein said eliciting an immune response is treating and/or preventing HPV16-related inappropriate cellular proliferation.

Embodiment 20: The vaccine or vector for use of embodiment 18 or 19, wherein said human subject is infected with at least one HPV16-related virus strain.

Embodiment 21: The vaccine or vector for use of any one of embodiments 18 to 20, wherein said eliciting an immune response is therapeutic vaccination, preferably against a HPV16-related virus-positive precancerous lesion or cancer.

Embodiment 22: The vaccine or vector for use of any one of embodiments 18 to 21, wherein the HLA molecules expressed by said human subject are unknown.

Embodiment 23: The vaccine or vector for use of any one of embodiments 18 to 22, wherein said subject is known or suspected to express at least one HLA of HLA supertypes selected from the list consisting of HLA-A01, HLA-A02, HLA-A3/A11, HLA-A24, HLA-B07, HLA-B15, HLA-B07, and HLA-B15.

Embodiment 24: A vaccine for use in therapeutic vaccination of a human subject against a human papillomavirus 16 (HPV16)-related virus, wherein said vaccine provides at least one discrete immunization peptide consisting of an amino acid sequence selected from at least four of the following groups:
(i) SEQ ID NOs: 1 to 10;
(ii) SEQ ID NOs:11 to 52;
(iii) SEQ ID NO:53 to 79;
(iv) SEQ ID NOs: 71 and 80 to 96;
(v) SEQ ID NOs:25, 81, and 97-106; and
(vi) SEQ ID NO: 9, 12, 23, 63, 81, 85, 101, and 107 to 131.

Embodiment 25: A vaccine for use in therapeutic vaccination of a human subject against a human papillomavirus 16 (HPV16)-related virus, wherein said vaccine provides at least four discrete immunization peptides consisting of amino acid sequences selected from at least four of the following groups:
(i) SEQ ID NOs: 1 to 10;
(ii) SEQ ID NOs:11 to 52;
(iii) SEQ ID NO:53 to 79;
(iv) SEQ ID NOs: 71 and 80 to 96;
(v) SEQ ID NOs:25, 81, and 97-106; and
(vi) SEQ ID NOs: 9, 12, 23, 63, 81, 85, 101, and 107 to 131; and
wherein said vaccine is a human leukocyte antigen (HLA) universal vaccine for use in any human subject expressing at least one HLA of a HLA supertype selected from the list consisting of HLA-A01, HLA-A02, HLA-A03/A11, HLA-A24, HLA-B07, and HLA-B15, and wherein each of said at least five immunization peptides, when presented as an HLA-complex, activates human anti-immunization peptide T cells.

Embodiment 26: The vaccine of embodiment 25, wherein each of said at least four immunization peptides, when presented as a HLA-complexes, activate human anti-immunization peptide T cells.

Embodiment 27: The vaccine for use of any one of embodiments 24 to 26, wherein at least one of said at least four immunogenic peptides consists of an amino acid sequence selected from group (ii) or group (iii).

Embodiment 28: The vaccine for use of any one of embodiments 24 to 27, wherein said vaccine provides at least five discrete immunization peptides consisting of amino acid sequences selected from five of said groups, preferably provides at least six discrete immunization peptides consisting of amino acid sequences selected from all six of said groups.

Embodiment 29: The vaccine for use of any one of embodiments 24 to 28, wherein said population is an average caucasoid, oriental, mixed, amerindian, african, or asian population, preferably with an HLA allele frequency as specified in Table 3.

Embodiment 30: The vaccine for use of any one of embodiments 24 to 29, wherein said T cell is a CD8+ T cell, more preferably a cytotoxic CD8+ T cell.

Embodiment 31: The vaccine for use of any one of embodiments 25 to 30, wherein,
(i) in case the immunization peptide is selected from SEQ ID NOs:1 to 10, the HLA supertype is HLA-A01;
(ii) in case the immunization peptide is selected from SEQ ID NOs:11 to 52, the HLA supertype is HLA-A02;
(iii) in case the immunization peptide is selected from SEQ ID NO:53 to 79, the HLA supertype is HLA-A03/A11;
(iv) in case the immunization peptide is selected from SEQ ID NOs: 71 and 80 to 96, the HLA supertype is HLA-A24;
(v) in case the immunization peptide is selected from SEQ ID NOs:25, 81, and 97-106, the HLA supertype is HLA-B07; and
(vi) in case the immunization peptide is selected from SEQ ID NO: 9, 12, 23, 63, 81, 85, 101, and 107 to 131, the HLA supertype is HLA-B15.

Embodiment 32: The vaccine for use of any one of embodiments 25 to 31, wherein said human subject is suffering from a HPV16-related virus-positive precancerous lesion or cancer.

Embodiment 33: The vaccine for use of embodiment 32, wherein a HPV16-derived peptide consisting of the same amino acid sequence as at least one of said immunization peptides is presented by HPV16-related virus-positive infected or cancer cells and wherein said at least one immunization peptide is selected from at least one of the following groups:
(i) SEQ ID NOs: 1 to 2;
(ii) SEQ ID NOs:11 to 23;
(iii) SEQ ID NOs:53 to 57;
(iv) SEQ ID NOs: 80 to 81;
(v) SEQ ID NOs: 81, 97, and 98; and
(vi) SEQ ID NOs: 81, and 107-114.

Embodiment 34: The vaccine for use of any one of embodiments 24 to 33, further comprising at least one feature of any one of embodiments 1 to 23.

Embodiment 35: A method of inducing an immune response to a HPV16-related virus in a subject, comprising contacting said subject with a vaccine according to any one of embodiments 1 to 15 or a vector according to embodiment 16.

Embodiment 36: Use of a vaccine according to any one of embodiments 1 to 15 or a vector according to embodiment 16 in the manufacture of a medicament, preferably a vaccine.

Embodiment 37: A kit comprising the vaccine according to any one of embodiments 1 to 15 or a vector according to embodiment 16 comprised in a housing.

Embodiment 38: The kit of embodiment 37, further comprising a means of administration.

Embodiment 39: A device comprising the vaccine according to any one of embodiments 1 to 15 or a vector according to embodiment 16.

Embodiment 40: A method for stimulating T cells recognizing HPV16-related virus-infected host cells (anti-HPV16 T cells), said method comprising
(I) contacting an agent expressing MHC class I molecules with a vaccine according to any one of embodiments 1 to 15 or a vector according to embodiment 16, thereby causing presentation of at least one of said immunization peptides on said MHC class I molecules,
(II) contacting the agent expressing MHC class I molecules of step (i) with T cells, and, thereby, stimulating anti-HPV16 T cells.

Embodiment 41: A method of producing T cells recognizing HPV16-infected host cells (anti-HPV16 T cells), comprising (i) contacting a subject with a vaccine according to any one of embodiments 1 to 16 or a vector according to embodiment 16 and (ii) obtaining anti-HPV16 T cells.

Embodiment 42: An anti-HPV T cell produced according to the method of embodiment 41 for use in treating and/or preventing HPV16-related disease.

Embodiment 43: A method for treating a HPV16-related infection or a HPV16-related virus positive inappropriate cellular proliferation in a subject, comprising
(I) administering a vaccine according to any one of embodiments 1 to 15 or a vector according to embodiment 16 to said subject; and
(II) thereby treating a HPV16-related infection or treating and/or preventing a HPV16-related virus positive inappropriate cellular proliferation.

Embodiment 44: The subject matter of any one of claims 18 to 43, wherein said HPV16-related HPV is selected from the list consisting of HPV16, HPV31, HPV33, HPV35, HPV52, HPV58, and HPV67, preferably is HPV16.

Embodiment 45: A method of any of the preceding embodiments relating to a method, wherein said method is an in vitro method.

Embodiment 46: The subject matter of any one of embodiments 1 to 45, wherein the vaccine does not comprise a mixture of six discrete and non-identical peptides comprising each exactly one amino acid sequence of SEQ ID NOs:1, 13, 54, 81, 97, and 109, preferably does not comprise the subject matter of the filed claims of the parallel application with attorney docket number DK16917PC, application number [to be provided], filed the same day.

All references cited in this specification are herewith incorporated by reference with respect to their entire disclosure content and the disclosure content specifically mentioned in this specification.

The following Examples shall merely illustrate the invention. They shall not be construed, whatsoever, to limit the scope of the invention.

### EXAMPLE 1: METHODS FOR T CELL REACTIVITY ASSESSMENT

### Interferon-γ ELISpot Assay

Prior to the ELISpot assay, ELISpot plates (Millipore Multiscreen-HA membrane sterile plate) were coated with 100 μl of 2 μg/mL anti-human IFN-γ (1-D1K) antibody in sterile PBS and incubated at 4° C. overnight or up to 3 days.

Before setting up the ELISpot assay, the coating antibody was discarded, wells were washed three times with 200 μL sterile PBS and blocked with 200 μL ELISpot medium at standard culture conditions for 1-1.5 h. After blocking, ELISpot medium was discarded and wells were filled with 100 μL antigen solution.

Each (short-term) T cell line (see below) was stimulated in a total of 8 wells: four wells with respective antigen (10 μg/mL single peptides, 0.1% DMSO or CEF peptide pool (1 μg/mL of each peptide)), two wells with concanavalin A (2 μg/ml) as unspecific mitogen-stimulated positive control, and two wells with 0.1% (v/v) DMSO as background control. Short-term antigen-specific T cell lines (described in 1.4) were added in a concentration of 1-2×105 cells in 100 μL per well (total volume of 200 μL/well). The cell numbers of three representative wells (CEF-, DMSO-, peptide-stimulated) were assessed, to enable the calculation of spot forming units (SFU) per 1×106 cells later on. The ELISpot plates were incubated at standard culture conditions for 20-24 h. Moving the plates or the incubator was strictly avoided during incubation to prevent blurred spot formation.

The remaining cell suspension was discarded and ELISpot plates were washed twice with PBS and twice with ELISpot washing buffer. All washes were performed with 200 μL/well. Then, 100 μL/well of 1 ng/mL sterile anti-human IFNγ biotin (7 B6-1) antibody in sterile PBS were added. After two hours of incubation in the dark at room temperature, the antibody was discarded and the plates were washed four times with ELISpot washing buffer. Sterile streptavidin-alkaline phosphatase was diluted by a factor of 1:2000 in sterile PBS and 100 μL/well of this dilution were distributed.

After 1.5 hours of incubation in the dark at room temperature, the antibody was discarded and the plates were washed four times with ELISpot washing buffer. Then, the BCIP/NBT-Plus substrate was filtered through a 0.22 μm filter and 100 μL/well were distributed. The plates were covered with aluminum foil and left at room temperature for 18-20 min until spots developed in the positive control wells. The reaction was stopped by washing the ELISpot plates with tap water and the plates were allowed to air dry for a minimum of 1.5 h.

### Analysis of ELISpot Plates

In order to obtain objective results about the number of SFU per well, a CTL automated ELISpot plate reader was used to count the ELISpot plates. The SmartCount™ counting mode of the ImmunoSpot 5.1.36 Professional DC software was used for the analysis. The settings were adjusted for each donor individually to account for donor specific variance. In general, the background balance was set to values between 0 and 10 and the spot separation was set to values between 1 and 4. The automatically determined minimum spot size was adjusted to exclude small background spots based on negative control wells. Similarly, maximum spot size was automatically determined based on positive control wells. A quality control for each well was done manually to detect and correct errors of the counting algorithm caused by uneven development or fibers. Based on the counting results, the mean SFU per 1×10⁶ cells and the stimulation index (SI), defined as the fold change of the mean SFU of peptide-stimulated relative to DMSO background wells, were calculated for each peptide-specific cell line. Responses with SI>2 and SFU per 1×10⁶ cells >100 as well as responses with SI>4 and SFU>50 were considered positive.

### T Cell Phenotyping and Intracellular Cytokine Staining

To characterize and quantify the responsive cells upon peptide stimulation, the peptide-specific T cell cultures with positive ELISpot results were further analyzed with T cell phenotyping and intracellular cytokine staining (ICS) assessed by flow cytometry.

The cells of the respective continued short-term T cell culture as well as the DMSO-stimulated and an unresponsive T cell line were harvested into 2 mL reaction tubes and centrifuged at 400×g for 10 min. The supernatant was discarded and the pellet of the unresponsive T cell line was resuspended in 100 μL ELISpot medium and used as unstimulated and unstained control. The pellets of the responsive and the DMSO-stimulated T cell lines were resuspended in 200 μL ELISpot medium and split equally into two wells of a V-bottom 96-well plate. One well of the responsive T cell lines was re-stimulated with 10 μg/mL of its specific peptide while the other well was mock stimulated with 0.1% (v/v) DMSO. As positive control, one well of the DMSO-stimulated T cell line was activated by treatment with 20 ng/ml Phorbol 12-myristate 13-acetate and 1 μM lonomycin. Then, 1:10 diluted GolgiStop (containing monensin) and 1:15 diluted GolgiPlug (containing brefeldin A) were added to all wells to inhibit intracellular transport processes. Monensin inhibits the trans-Golgi transport, while brefeldin A inhibits transport between the ER and the Golgi (Chardin and McCormick, 1999; Mollenhauer et al., 1990). The stimulated cells were incubated for 5 h at standard culture conditions.

After incubation, the cells were resuspended, centrifuged at 400×g for 5 min and the supernatant was discarded. Then, the cells were resuspended in 50 μL cold staining buffer (unstained) or 50 μL cold staining buffer containing the phenotyping antibodies against CD3, CD4, CD8 and the LIVE/DEAD™ Fixable Near-IR Dead Cell Stain (unstimulated and stimulated) and incubated at 4° C. for 30 min. After the surface staining, the cells were washed twice with 200 μL staining buffer and centrifugation at 1400 rpm, followed by fixation with 1% paraformaldehyde (PFA) solution for 15 min at 4° C. All following centrifugation steps were carried out at 1400 rpm for 5 min. After fixation, the cells were again washed twice with 200 μL cold staining buffer and were then resuspended in 100 μL 1× perm/wash buffer diluted in PBS (provided is 10×) for 15 min at 4° C. for cell membrane permeabilization. Subsequently, the plate was centrifuged and the supernatant was discarded. The cells were resuspended in 50 μL perm/wash buffer diluted in PBS (unstained) or 50 μL perm/wash buffer containing the intracellular cytokine antibodies against IFNγ, TNFα and granzyme B (unstimulated and stimulated) for 30 min at 4° C. After intracellular cytokine staining, the cells were washed twice with 200 μL 1× perm/wash buffer. Then 100 μL fix/perm solution (provided in the BD Cytofix/Cytoperm kit) were added per well. After 20 min of incubation at 4° C., the cells of each well were washed twice with 200 μL 1× perm/wash buffer before being resuspended in 100 μL staining buffer and stored at 4° C. overnight.

OneComp eBeads™ (eBeads) and ArC™ Amine Reactive Compensation beads (ArC beads) were stained as compensation controls. For each antibody, 50 μL of the eBeads suspension were stained with the same dilution as used for staining the cells. The ArC beads were stained with 1 μL LIVE/DEAD™ Fixable Near-IR Dead Cell Stain stock solution in 50 μL staining buffer. The beads were incubated at 4° C. for 30 min and were then washed two times with 1 mL staining buffer and centrifugation at 400×g. Finally, the stained beads were resuspended in 400 μL staining buffer, one drop of ArC™ negative beads was added to the labelled ArC beads, and all beads were stored at 4° C. overnight.

On the next day, the samples and the single stained compensation controls were acquired at a BD FACS Canto™ II analyzer using the BD FACS Diva Software Version 6. The obtained data were analyzed with the FlowJo software Version 10 by applying the same gating strategy to all samples.

### Short-Term T Cell Lines

Short-term antigen-specific T cell lines were generated from PBMCs in order to expand antigen-specific memory T cells induced by previous natural HPV16 infections. To do so, T cells were stimulated with HLA-matched HPV16 E6- or E7-derived HLA-ligands. To set up short-term T cell lines, human PBMCs were thawed and subsequently resuspended in T cell medium supplemented with recombinant human interleukin-7 (rhIL-7) (10 ng/mL) and recombinant human interleukin-15 (rhIL-15) (20 ng/mL). The cells were counted and 1-2×10⁶ cells per antigen stimulation were seeded in wells of 24-well plates in a total volume of 2 mL per T cell line. To obtain antigen-specific T cell lines, 10 μg/mL of HLA-binding HPV16 E6/E7-derived peptides were added to each well. As peptide-specific positive control, one cell line was stimulated with a peptide pool consisting of 23 well described HLA class I-restricted epitopes of the widespread viruses human cytomegalovirus (CMV), Epstein-Barr-Virus (EBV) and Influenza A (CEF peptide pool) (Currier et al., 2002) at a concentration of 1 μg/mL for each peptide. As single peptide controls, two cell lines were stimulated with 10 μg/mL HLA-matched EBV- and CMV-derived peptide. As peptide-specific negative control, a cell line was stimulated with 10 μg/mL HLA-matched human immunodeficiency virus (HIV)-derived peptide (10 μg/mL), while treatment with 0.1% (v/v) dimethyl sulfoxide (DMSO, peptide solvent) served as unspecific negative control. The stimulated PBMCs were cultivated at standard culture conditions.

On day 3, the cells were fed with recombinant human interleukin-2 (rhIL-2) (20 U/mL) and rhIL-15 (20 ng/mL) to promote proliferation of T cells. Seven days after culture setup, a half-medium change was performed. For this, the cells were pelleted on the well bottom by centrifugation at 300×g for 5 min and 1 mL of the supernatant was carefully removed from each well. Then, 1 ml fresh T cell medium supplemented with rhIL-2 (20 U/mL final) and rhIL-15 (20 ng/mL final) was added and cells were resuspended.

After 12 days of culturing, half of the cells of each culture was used to set up ELISpot assays as described in 1.1. The remaining cells were fed with 1 mL ELISpot medium and cultivated for two more days until the cells were used for T cell phenotyping and intracellular cytokine staining (ICS) as described in 1.2.

### Cytotoxicity Assay

The flow cytometry based VITAL FR cytotoxicity assay was used as published by Stanke et al. (Stanke et al., 2010) to assess whether peptide-specific T cells are able to specifically kill HPV-transformed target cells. Briefly, two HLA-A2+ cell lines, a HPV16-negative (HPV16−) control cell line (C33A) and a HPV16-positive (HPV16+) target cell line (CaSki) were fluorescently labeled with either FarRed or CFSE and were co-incubated with peptide-specific CD8+ T cells as effector cells. After 48 h co-incubation, the fluorescently labeled cells were analyzed by flow cytometry to determine the surviving cells and calculate the specific cytolytic function of the effector cells by the ratio of target and control cells.

CaSki and C33A cells were fluorescently labeled with CFSE and FarRed, respectively. Cells of both cell lines were harvested and resuspended in plain RPMI medium at a concentration of 1×10⁶ cells/mL. For cell labeling, 5 μM CSFE were added to CaSki cells and 0.25 μM FarRed were added to C33A cells. The cells were incubated at 37° C. and the reaction was stopped after 10 min by addition of 50 mL RPMI with 10% FBS. The cells were centrifuged at 300×g for 5 min, the supernatant was discarded and the cells were resuspended in their cell culture medium. The labeled cells were seeded in T25 cell culture flasks with 1-2×10⁶ cells/flask.

On the next day, the labeled CaSki and C33A as well as the epitope-specific semi-long-term T cell lines (described in 1.6) were harvested. CD8+ T cells were isolated from the epitope-specific semi-long-term T cell lines using the MACS CD8+ T cell isolation kit (Miltenyi Biotec) according to the manufacturer's protocol for LS columns. The CD8+ T cells were resuspended in T cell medium and added to wells of a F-bottom 96-well plate in serial dilutions to achieve triplicates of 6×10⁴, 3×10⁴, 1.5×10⁴, 0.75×10⁴ and 0.375×10⁴ cells per well. The CaSki and C33A cells were mixed 1:1 in T cell medium and 3×10³ cells were added to each well containing T cells. Thereby effector:target (E:T) ratios of 1:20, 1:10, 1:5, 1:2.5 and 1:1.25 were achieved. Additionally, triplicates of mixed target cells without T cells were seeded, which is referred to as E:T 0. After 48 h incubation at 37° C., 5% C02, the cells were harvested and analyzed at a BD FACS Canto™ II analyzer using the BD FACS Diva Software Version 6. The obtained data were analyzed with the FlowJo software Version 10 by applying the same gating strategy to all samples. Subsequently, the equations (1) and (2) given below were applied in Excel (Microsoft Office 2016) to calculate specific killing.

$$ratio of target cells ( \% ) = \frac{frequency of CaSki}{frequency of C 33 A} \qquad ( 1 )$$

$$specific killing ( \% ) = 100 - ( \frac{ratio of target cells in presence of T cells}{ratio of target cells without T cells} * 100 ) \qquad ( 2 )$$

### Semi Long-Term T Cell Lines

Semi long-term T cell lines were set up to obtain high numbers of T cells, which are required for the analysis of epitope specific cytotoxic T cell responses in VITAL FR cytotoxicity assays. Autologous DCs were generated from PBMCs as described in 1.7 and loaded with the desired HPV16 peptide. PBMCs of the same donor were thawed and resuspended in T cell medium supplemented with rhIL-7 (10 ng/mL) and rhIL-15 (20 ng/mL). 1×10⁷ PBMCs/well were seeded to a 24-well plate in a total volume of 1 mL/well. Peptide-loaded DCs were added in a ratio of 50:1 (PBMCs:DCs) in 1 mL rhIL-7 (10 ng/mL) and rhIL-15 (20 ng/mL) supplemented T cell medium to achieve a final volume of 2 mL. The cell cultures were fed with rhIL-2 (final concentration: 40 U/mL) every other day. If the medium turned yellow the feeding was combined with a half medium change with 1 mL fresh IL-2 supplemented T cell medium. After eight days, the peptide stimulation with peptide-loaded DCs was repeated and cultivation was continued. On day 15, the T cell lines were harvested and used for CD8+ T cell isolation and cytotoxicity assays.

### Generation of Autologous DCs

To generate autologous DCs, frozen human PBMCs were thawed. After thawing, cells were counted and resuspended in DC medium to reach a density of 5×10⁶ cells/mL. From this suspension, 1×10⁷ cells in 2 mL DC medium were seeded per well in a 6-well plate.

After 3 hours incubation at 37° C., 5% C02 the supernatant was removed and transferred to a 50 mL tube. The wells were gently washed with 1 mL warm DC medium to remove non-adherent cells and the washing medium was collected together with the supernatant. The remaining adherent cells were fed with 2 mL fresh DC medium supplemented with 100 ng/mL granulocyte-macrophage colony-stimulating factor (GM-CSF) and 50 ng/mL interleukin-4 (IL-4) for further cultivation. The collected non-adherent cells containing T and B cells were counted and frozen for future use.

After three days of culturing, the cells were fed with 0.5 mL fresh DC medium supplemented with 100 ng/mL GM-CSF and 50 ng/mL IL-4. Six days after the cultures were started, the DCs were maturated by adding 1000 U/ml tumor necrosis factor α (TNFα), 10 ng/ml interleukin-1β (IL-1β), 10 ng/ml interleukin-6 (IL-6), 1 μM Prostaglandin E2 (PGE2) and 1 μg/ml lipopolysaccharide (LPS) to each well. The cells were incubated with this maturation cocktail for 48 hours and harvested by gentle scraping with a cell scraper.

### Live-Cell Imaging-Based Cytotoxicity Assay

In addition to the VITAL FR assay described herein above, a newly established live-cell imaging-based cytotoxicity assay was applied. In this assay, transiently red labeled HPV16 transformed target cells were co-incubated with either HPV16-peptide-specific or non-specific CD8+ T cells. Apoptotic cells were stained with a green caspase dye. By live cell imaging, the frequency of apoptotic target cell (stained in red and green) was analyzed over time. Peptide-specific killing was detected by increased frequencies of apoptotic target cells in co-culture with HPV16-peptide-specific CD8+ T cells compared to co-culture with non-specific CD8+ T cells. This highly sensitive assay was used to detect killing mediated by low frequent CTL populations and, in contrast to the common endpoint assays, it enables the time course analysis of the CTL mediated cytotoxicity. Moreover, it is very flexible and can easily be adapted for other target cell lines of interest.

### EXAMPLE 2: IMMUNOPRECIPITATION (IP) AND LC/MS ANALYSIS OF PEPTIDES PRESENTED BY CULTURED CELLS

### Immunoprecipitation of HLA-Presented Peptides

HLA immunoprecipitation (IP) was performed according to previously published protocols (Bassani-Sternberg et al., 2016; Chong et al., 2018). Briefly, HPV16+ cells were lysed with a lysis buffer containing 1% N-octyl-β-D glucopyranoside, 0.25% Na-Deoxycholate, protease inhibitor cocktail (Sigma-Aldrich, Mannheim, Germany)/PMSF (Carl Roth, Karlsruhe, Germany) in PBS. After centrifugation at 40,000×g, 4° C., for 30 min, HLA-peptide complexes were immuno-precipitated by incubation with mouse anti-human HLA-A,B,C monoclonal antibody (clone W6/32, Biolegend, San Diego, CA, USA) or in some cases HLA-type-specific antibody (HLA-A2: clone BB7.2, HLA-A3: clone GAP A3, HLA-A11/A24: clone Hb 164, HLA-B7: clone BB7.1) crosslinked to protein G Sepharose beads (Cytiva, Marlborough, MA, USA) or Protein A—Sepharose™ 4B (Invitrogen Corporation, Camarillo, CA, USA) for 4 h at 4° C. under constant mixing on a rotating wheel. Supernatant was discarded after centrifugation at 3200×g, for 3 min at RT. Pelleted HLA-peptide complexes bound to antibody-beads were washed 3 times in each of the following steps: first with ice-cold 20 mM Tris-HCl pH 8 containing 150 mM NaCl followed by the same buffer but with 400 mM NaCl and finally with 20 mM Tris-HCl alone. Peptides were eluted from antibody-beads bound to HLA by 0.3% TFA. Alkylation of cysteine-containing peptides was optionally performed at this stage. 1 ml of IP eluate was reduced with 100 μl HEPES/50 mM TCEP with incubation for 10 min at RT. Alkylation was performed with 100 μl of 400 mM iodoacetamide (IAA) with incubation in the dark for 20 min at RT. Alkylation was quenched with 100 μl of 1 M HEPES/50 mM TCEP with incubation for 3 min at RT. The sample was acidified again with 60 μl of 10% TFA. Resulting peptides were desalted by reverse-phase purification using a SepPak 96-well plate (Waters, Milford, MA, USA). While loaded on the sorbent, an oxidation reaction was performed. Performic acid was prepared by combining 5 μl of 30% H₂O₂ and 45 μl of 10% formic acid in a glass vial and letting it rest for 5 min. Sample oxidation was performed by brief application of 1 ml of the performic acid solution in the well, which was washed out afterwards with 1 ml of 0.1% TFA. Peptides eluted in 28% ACN in 0.1% TFA were dried by vacuum centrifugation (Concentrator plus, Eppendorf, Hamburg, Germany).

### Direct Infusion and LC-MS

The set of predicted target peptides were acquired as stable isotope labelled (SIL) peptides from commercial suppliers (crude synthesis product; isotope purity >99 atom % 13C and 15N). To alkylate cysteine-containing peptides, 2 ng per peptide of vacuum dried peptide mixture was resolubilized in 20 μl 100 mM HEPES with 3 min sonication in a 0.5 μl protein LoBind microcentrifuge tube. 5 μl (500 pmol per peptide) of the sample was reduced by addition of 2 μl HEPES/50 mM TCEP with incubation for 10 min at RT. Alkylation was performed by addition of 2 μl 400 mM IAA with incubation in the dark for 20 min at RT. Alkylation was quenched by addition of 2 μl 100 mM HEPES/50 mM TCEP. The sample was acidified by adding 0.3% TFA to a volume of 500 μl and desalted using a 100 mg sorbent well of a 96-well SepPak plate (Waters, Milford, MA, USA). The elution step was performed with 80% ACN/0.1% TFA.

First, analysis by direct infusion was performed in order to optimize normalized collision energy (NCE) for each single peptide in a mixture. NCE values were ranging from 4% to 42% and each value, in 2% steps, was measured 4 times for four charge states (1+ to 4+) of each peptide. Orbitrap Exploris 480 (Thermo Fisher Scientific) in targeted MS2 scan (PRM) was operated using 30K resolution at 200 m/z, standard automated gain control (AGC) target, 350 ms maximum injection time (IT), 1 micro scan in centroid data acquisition mode. For all measurements, isolation window was 0.4, 0.7, 1 or 1.5 m/z, depending on the mass of the precursor, as recommended by the manufacturer. Ion chromatograms of all expected transitions were extracted with 12 ppm mass tolerance from the centroided spectra and deconvoluted using purpose-made R scripts. The best NCE value was selected so that the intensity of any 5th most intense transitions was maximized, thereby maximizing the chance of detecting at least 5 transitions. The retention time (RT) for each peptide was determined by LC-MS in DDA mode with an inclusion list of target peptides in all four charge-states. The MS resolution was set to 120K, 3e6 AGC target, 50 ms maximum IT and the LC operated as described below.

For LC-MS, samples were dissolved in 2.5 to 5 μl of 5% ACN in 0.1% TFA with 50 fmol of Peptide Retention Time Calibration (PRTC) Mixture (88321, Pierce™) spiked in, followed by 3 min bath sonication. All samples were analysed by liquid chromatography (U-3000, Thermo Fisher Scientific) coupled to Orbitrap Exploris 480 (Thermo Fisher Scientific). 150 fmol of BSA digest (88341, Pierce™)) and 50 fmol PRTC Mixture (free of target peptides) was systematically injected before IP samples as a negative control to test for the absence of any artefact, e.g. due to possible carryover of light contamination from SIL peptides after prior quality control tests (Salek et al. 2022). The LC gradient consisted of multiple segments. First, the solvent B (100% ACN, 0.1% FA) went from 2% to 6% and 94% A (0.1% FA in H2O) in 5 min and then to 28.5% B in 75 min. It reached 80% in 4 min followed by 5 min wash at 80% B. Finally, the column was equilibrated for 11 min at 2% B.

For LC-MS experiments, the MS was acquired with 60K resolution at 200 m/z, over the mass range from 150 to 1450 m/z, 3e6 AGC target and 25 ms maximum IT. MS2 data was acquired with PRM scans using 60 k or 120 k resolution at 200 m/z. Target precursor list was provided with preselected charge states, the corresponding m/z and optimized collision energy values for each target, their expected retention time (+/−1 to 3 min) pre-defined with SIL peptide. The normalized AGC target was set to 1000% (or 1e6). The maximum injection time mode was set to dynamic, allowing sampling of a minimum of 5 points across the chromatographic peak. The dynamic RT feature using the Pierce PRTC mixture was active.

### Data Analysis

LC-MS data was analysed with the Skyline software v. 20.2 (MacLean et al., 2010). A minimum of top 5 intensity product ions were extracted with 7 ppm mass tolerance. Detected peaks were manually curated. Light peaks were discarded when peak retention times or shapes did not match with the heavy reference, when the normalized spectral contrast angle (NSCA) (Toprak et al., 2014) was low, or when too few transitions were detected.

The findings of the present invention are summarized also in Table 2 below; as shown, there are a vast number of peptides derivable from HPV 16 E6 and E7 (approx. 9000). Prediction algorithms identify approx. ⅓ of the peptides actually binding to HLA, and wrongly predict binders in about 50% of cases. Moreover, of immunogenic peptides only ½ are correctly predicted, and of peptides actually presented on HPV16 positive tumor cells, only approx. ¼ was correctly predicted.

Of the peptides in Table 1, peptides found to have the highest rates of immunoactivation in PBMC samples, to induce the strongest CD8 T cell responses, and/or shown to be presented as HLA-complexes by the highest numbers of HPV16 positive cell lines were SEQ ID NOs:1, 2, 11, 13, 14, 17, 18, 53, 54, 55, 80, 81, 82, 97, 98, 107, 108, and 109.

HLA-supertype frequencies per ethnicity are shown in Table 3.

### Detection of Cystein-Containing Peptides

For the detection of HPV16-derived T-cell epitopes, an immunopeptidomics workflow was established for which each individual candidate epitope is assessed by a scientist and an optimized targeting strategy is devised. Due to HPV16 proteins E6 and E7 being rich in cysteine, 148 of the 242 candidate peptides contained cysteine (cf. Table 4 below). Contrary to the usual proceeding in the art, cf. e.g. Blatnik et al. ((2018, loc.cit.)), cysteine-containing peptides were not excluded from the analysis. This required an adapted strategy as the detection of cysteine-containing peptides is particularly challenging for MS experiments due to the propensity of the thiol side chain to undergo oxidative modifications. The resulting dispersion of cysteine-containing peptides into various forms does not allow for the required sensitive detection in targeted experiments. For this reason, the preceding studies on HPV16 did not target any cysteine-containing peptides and this limitation was overcome by the integration of a cysteine alkylation reaction into the experiment.

TABLE 4
Numbers of cysteine-containing candidate peptides.

| | Candidate peptide count | |
|---|---|---|
| Total | 242 | |
| Not cysteine-containing | 148 | (61%) |
| Cysteine-containing | 94 | (39%) |

TABLE 1
Preferred Peptide/HLA combinations; Protein/Position: HPV16 protein and amino acid position the peptide is derived from, deviations from
the HPV16 reference sequence are indicated if applicable; presentation: HLA-presentation of the peptide by HPV16-positive cells shown by MS;
ELISPOT: Immungenicity of the peptide shown by IFN-gamma ELISPOT analysis; cytokine: Immunogenicity of the peptide shown by intracellular
cytokine staining (IFN-gamma, TNF-alpha); cytotox: Ability of peptide-specific T cells to kill HPV16-positive cells. *highlights data from
published resources.
Table 1 (i) Peptides presented via HLA-A01

| | | SEQ ID | HLA | | | | |
|---|---|---|---|---|---|---|---|
| Protein/Position | Peptide sequence | NO | restriction | ELISpot | cytokine | cytotox | presentation |
| E6/80-88 | ISEYRHYCY | 1 | A01 | +* | | | + |
| E7/2-11 | HGDTPTLHEY | 2 | A01 | +* | | | + |
| E6 E36Q/29-39 | TIHDIILQCVY | 3 | A01 | + | + | | |
| E6 A68G/67-77 | YGVCDKCLKFY | 4 | A01 | + | + | | |
| E6 H85Y/82-91 | EYRYYCYSLY | 5 | A01 | + | + | | |
| E7/14-23 | DLQPETTDLY | 6 | A01 | + | + | | |
| E7/19-27 | TTDLYCYEQ | 7 | A01 | + | + | | |
| E6/78-88 | SKISEYRHYCY | 8 | A01 | + | | | |
| E6/81-91 | SEYRHYCYSLY | 9 | A01 | + | | | |
| E7/18-25 | ETTDLYCY | 10 | A01 | + | | | |
| E7/19-28 | TTDLYCYEQL | 132 | A01 | + | | | |
| E6/81-88 | SEYRHYCY | 133 | A01 | + | | | |
| E6 D32E/30-39 | IHEIILECVY | 134 | A01 | + | | | |
| E6 A68G/68-77 | GVCDKCLKFY | 135 | A01 | + | | | |

Table 1 (ii) Peptides presented via HLA-A01

| Protein/Position | Peptide sequence | SEQ ID NO | HLA restriction | ELISpot | cytokine | cytotox | presentation |
|---|---|---|---|---|---|---|---|
| E6/29-38 | TIHDIILECV | 11 | A02 | +* | +* | +* | +* |
| E7/7-15 | TLHEYMLDL | 12 | A02 | +* | * | +* | +* |
| E7/11-19 | YMLDLQPET | 13 | A02 | +* | * | +* | +* |
| E7/11-20 | YMLDLQPETT | 14 | A02 | +* | +* | +* | +* |
| E7/11-21 | YMLDLQPETTD | 15 | A02 | +* | | + | +* |
| E7/12-19 | MLDLQPET | 16 | A02 | +* | | + | +* |
| E7/66-74 | RLCVQSTHV | 17 | A02 | + | | + | + |
| E7/77-86 | RTLEDLLMGT | 18 | A02 | +* | * | * | + |
| E7/77-87 | RTLEDLLMGTL | 19 | A02 | +* | + | + | +* |
| E7/78-86 | TLEDLLMGT | 20 | A02 | +* | | | +* |
| E7/80-90 | EDLLMGTLGIV | 21 | A02 | +* | + | + | +* |
| E7/81-90 | DLLMGTLGIV | 22 | A02 | +* | | | +* |
| E7/82-90 | LLMGTLGIV | 23 | A02 | +* | * | +* | +* |
| E7/81-91 | DLLMGTLGIVC | 24 | A02 | + | + | +* | |
| E7/83-93 | LMGTLGIVCPI | 25 | A02 | + | + | + | |
| E6/18-26 | KLPQLCTEL | 26 | A02 | +* | * | * | |
| E6 Q21D/18-26 | KLPDLCTEL | 27 | A02 | +* | * | * | |
| E7/12-20 | MLDLQPETT | 28 | A02 | +* | * | * | |
| E7/85-93 | GTLGIVCPI | 29 | A02 | +* | +* | +* | |
| E7/86-93 | TLGIVCPI | 30 | A02 | +* | * | * | |
| E7/86-94 | TLGIVCPIC | 31 | A02 | +* | * | * | |
| E7/11-18 | YMLDLQPE | 32 | A02 | + | | + | |
| E7/84-93 | MGTLGIVCPI | 33 | A02 | + | | + | |
| E6 D32E/25-33 | ELQTTIHEI | 34 | A02 | + | + | | |
| E6 D32E/28-38 | TTIHEIILECV | 35 | A02 | + | + | | |
| E6 D32E, 134R/28-38 | TTIHEIRLECV | 36 | A02 | + | + | | |
| E6 D32E/29-38 | TIHEIILECV | 37 | A02 | + | + | | |
| E6 D32E, 134R/29-38 | TIHEIRLECV | 38 | A02 | + | + | | |
| E6 H85Y/81-90 | SEYRYYCYSL | 39 | A02 | + | + | | |
| E6 H85Y, L90V/81-90 | SEYRYYCYSV | 40 | A02 | + | + | | |
| E6 H85Y, L90V/83-90 | YRYYCYSV | 41 | A02 | + | + | | |
| E6 H85Y/84-93 | RYYCYSLYGT | 42 | A02 | + | + | | |
| E7/82-91 | LLMGTLGIVC | 43 | A02 | + | + | | |
| E7/82-92 | LLMGTLGIVCP | 44 | A02 | + | + | + | |
| E6 R171/9-17 | FQDPQERPI | 45 | A02 | + | | | |
| E6 R171/9-19 | FQDPQERPIKL | 46 | A02 | + | | | |
| E6/18-28 | KLPQLCTELQT | 47 | A02 | + | | | |
| E6/25-33 | ELQTTIHDI | 48 | A02 | + | | | |
| E6/28-38 | TTIHDIILECV | 49 | A02 | + | | + | |
| E6 H85Y/83-90 | YRYYCYSL | 50 | A02 | + | + | | |
| E7/7-17 | TLHEYMLDLQP | 51 | A02 | + | | | |
| E7/76-86 | IRTLEDLLMGT | 52 | A02 | + | | + | |

Table 1(iii) Peptides presented via HLA-A03/A11

| Protein/Position | Peptide sequence | SEQ ID NO | HLA restriction | ELISpot | cytokine | cytotox | presentation |
|---|---|---|---|---|---|---|---|
| E6/37-46 | CVYCKQQLLR | 53 | A03 | + | | | + |
| | | | A11 | * | | | + |
| E6/93-101 | TTLEQQYNK | 54 | A03 | + | | | |
| | | | A11 | * | | | + |
| E7/89-97 | IVCPICSQK | 55 | A03 | | | | + |
| | | | A11 | * | | | |
| E6/33-41 | IILECVYCK | 56 | A11 | - | | | + |
| E6/125-133 | HLDKKQRFH | 57 | A03 | - | | | + |
| E6 D32E/31-41 | HEIILECVYCK | 58 | A11 | + | + | | |
| E6/52-62 | FAFRDLCIVYR | 59 | A11 | + | + | | |
| E6/53-62 | AFRDLCIVYR | 60 | A11 | + | + | | |
| E6/59-67 | IVYRDGNPY | 61 | A3 | + | + | | |
| | | | A11 | + | + | | |
| E6/65-75 | NPYAVCDKCLK | 62 | A11 | + | + | | |
| E6/68-77 | AVCDKCLKFY | 63 | A03 | + | + | | |
| | | | A11 | +* | + | | |
| E6/68-78 | AVCDKCLKFYS | 64 | A11 | + | + | | |
| E6/69-79 | VCDKCLKFYSK | 65 | A11 | + | + | | |
| E7/88-97 | GIVCPICSQK | 66 | A03 | + | + | | |
| E6/8-18 | MFQDPQERPRK | 67 | A03 | + | | | |
| E6/67-77 | YAVCDKCLKFY | 68 | A11 | + | | | |
| E6/68-75 | AVCDKCLK | 69 | A03 | + | | | |
| E6/72-80 | KCLKFYSKI | 70 | A03 | + | | | |
| E6/75-83 | KFYSKISEY | 71 | A03 | + | | | |
| E6/75-84 | KFYSKISEYR | 72 | A03 | + | | | |
| E6 L90V/84-94 | RHYCYSVYGTT | 73 | A03 | + | | | |
| E6/105-115 | DLLIRCINCQK | 74 | A11 | + | + | | |
| E6/106-115 | LLIRCINCQK | 75 | A03 | +* | | | |
| E6/107-115 | LIRCINCQK | 76 | A03 | + | | | |
| E6/109-119 | RCINCQKPLCP | 77 | A03 | + | | | |
| E6/129-138 | KQRFHNIRGR | 78 | A03 | + | | | |
| E7/87-97 | LGIVCPICSQK | 79 | A11 | + | | | |
| E6/92-101 | GTTLEQQYNK | 136 | A03 | + | | | |
| E6/84-91 | RHYCYSLY | 137 | A03 | + | + | | |
| E6 A68G/68-77 | GVCDKCLKFY | 135 | A03 | + | | | NA |

Table 1 (iv) Peptides presented via HLA-A24

| Protein/Position | Peptide sequence | SEQ ID NO | HLA restriction | ELISpot | cytokine | cytotox | presentation |
|---|---|---|---|---|---|---|---|
| E6/38-45 | VYCKQQLL | 80 | A24 | - | | | + |
| E7/49-57 | RAHYNIVTF | 81 | A24 | - | | | + |
| E6/82-90 | EYRHYCYSL | 82 | A24 | +* | +* | * | |
| E7/56-65 | TFCCKCDSTL | 83 | A24 | + | | + | |
| E6/66-76 | PYAVCDKCLKF | 84 | A24 | + | + | | |
| E6 L90V/81-91 | SEYRHYCYSVY | 85 | A24 | + | + | | |
| E6 L90V/90-99 | VYGTTLEQQY | 86 | A24 | + | + | | |
| E7/47-57 | PDRAHYNIVTF | 87 | A24 | + | + | | |
| E7/48-57 | DRAHYNIVTF | 88 | A24 | + | + | | |
| E7/51-59 | HYNIVTFCC | 89 | A24 | + | + | | |
| E7 S63F/56-63 | TFCCKCDF | 90 | A24 | + | + | | |
| E6 A68G/60-69 | VYRDGNPYGV | 91 | A24 | + | | | |
| E6/75-83 | KFYSKISEY | 71 | A24 | + | | | |
| E6/85-95 | HYCYSLYGTTL | 92 | A24 | + | + | | |
| E6/88-95 | YSLYGTTL | 93 | A24 | + | | | |
| E6/125-135 | HLDKKQRFHNI | 94 | A24 | + | | | |
| E6/127-134 | DKKQRFHN | 95 | A24 | + | | | |
| E7/50-57 | AHYNIVTF | 96 | A24 | + | | | + |
| E7/51-58 | HYNIVTFC | 138 | A24 | + | | | |
| E7 S63F/56-65 | TFCCKCDFTL | 139 | A24 | + | | | NA |

Table 1(v) Peptides presented via HLA-B07

| Protein/Position | Peptide sequence | SEQ ID NO | HLA restriction | ELISpot | cytokine | cytotox | presentation |
|---|---|---|---|---|---|---|---|
| E6/15-22 | RPRKLPQL | 97 | B07 | + | + | | + |
| E7/49-57 | RAHYNIVTF | 81 | B07 | +* | | | + |
| E7/5-13 | TPTLHEYML | 98 | B07 | * | | | + |
| E6/15-25 | RPRKLPQLCTE | 99 | B07 | + | + | | |
| E6/95-103 | LEQQYNKPL | 100 | B07 | + | + | | |
| E6/134-144 | NIRGRWTGRCM | 101 | B07 | + | + | | |
| E7/46-55 | EPDRAHYNIV | 102 | B07 | + | + | | |
| E7/83-93 | LMGTLGIVCPI | 25 | B07 | + | + | | |
| E6/15-23 | RPRKLPQLC | 103 | B07 | +* | +* | | |
| E6/15-24 | RPRKLPQLCT | 104 | B07 | + | | | |
| E6/101-108 | KPLCDLLI | 105 | B07 | + | | | |
| E6/142-151 | RCMSCCRSSR | 106 | B07 | + | | | |
| E6 R17G/15-22 | RPGKLPQL | 140 | B07 | | | | + |
| E6 Q21D/15-24 | RPRKLPDLCT | 141 | B07 | + | + | | |
| E6E120D/118-126 | CPDEKQRHL | 142 | B07 | + | | | NA |
| E6/19-28 | LPQLCTELQT | 143 | B07 | + | | | |

Table 1 (vi) Peptides presented via HLA-B15

| Protein/Position | Peptide sequence | SEQ ID NO | HLA restriction | ELISpot | cytokine | cytotox | presentation |
|---|---|---|---|---|---|---|---|
| E6/41-50 | KQQLLRREVY | 107 | B15 | + | + | | + |
| E6/53-61 | AFRDLCIVY | 108 | B15 | + | + | | + |
| E7/15-23 | LQPETTDLY | 109 | B15 | +* | + | | + |
| E7/43-52 | GQAEPDRAHY | 110 | B15 | + | + | | + |
| E6/42-50 | QQLLRREVY | 111 | B15 | + | | | + |
| E7/49-57 | RAHYNIVTF | 81 | B15 | + | | | + |
| E6 L90V/89-99 | SVYGTTLEQQY | 112 | B15 | - | | | + |
| E7/15-24 | LQPETTDLYC | 113 | B15 | - | | | + |
| E7/15-25 | LQPETTDLYCY | 114 | B15 | - | | | + |
| E6/52-61 | FAFRDLCIVY | 115 | B15 | + | + | | |
| E6/57-67 | LCIVYRDGNPY | 116 | B15 | + | + | | |
| E6/68-77 | AVCDKCLKFY | 63 | B15 | + | + | | |
| E6 L90V/81-91 | SEYRHYCYSVY | 85 | B15 | + | + | | |
| E6 L90V/83-91 | YRHYCYSVY | 117 | B15 | + | + | | |
| E6/44-54 | LLRREVYDFAF | 118 | B15 | + | | | |
| E6/45-54 | LRREVYDFAF | 119 | B15 | + | | | |
| E6/59-69 | IVYRDGNPYAV | 120 | B15 | + | | | |
| E6/68-76 | AVCDKCLKF | 121 | B15 | + | | | |
| E6/73-83 | CLKFYSKISEY | 122 | B15 | + | | | |
| E6/76-84 | FYSKISEY | 123 | B15 | + | | | |
| E6/77-86 | YSKISEYRHY | 124 | B15 | + | | | |
| E6/78-86 | SKISEYRHY | 125 | B15 | + | | | |
| E6/81-88 | SEYRHYCY | 126 | B15 | + | | | |
| E6/81-91 | SEYRHYCYSLY | 9 | B15 | + | | | |
| E6/97-106 | QQYNKPLCDL | 127 | B15 | + | | | |
| E6/113-121 | CQKPLCPEE | 128 | B15 | + | | | |
| E6/122-132 | KQRHLDKKQRF | 129 | B15 | + | | | |
| E6/129-139 | KQRFHNIRGRW | 130 | B15 | + | | | |
| E6/134-144 | NIRGRWTGRCM | 101 | B15 | + | + | | |
| E7/7-15 | TLHEYMLDL | 12 | B15 | + | | | |
| E7/43-51 | GQAEPDRAH | 131 | B15 | + | | | |
| E7/82-90 | LLMGTLGIV | 23 | B15 | + | | | |
| E7/50-57 | AHYNIVTF | 144 | B15 | | | | + |
| E6/79-86 | KISEYRHY | 145 | B15 | + | | | |
| E6/136-144 | RGRWTGRCM | 146 | B15 | + | | | |
| E7/42-52 | AGQAEPDRAHY | 147 | B15 | + | | | |
| E6/76-83 | FYSKISEY | 148 | B15 | + | | | |

TABLE 2
Prediction vs. experimental confirmation of peptide functionality

| | | | num- |
|---|---|---|---|
| peptide type | number | peptide type | ber |
| possible binding peptides | approx. 9000 | | |
| predicted to bind to HLA A1, | 201 | | |
| A2, A3/A11, B07, or B15 | | | |
| experientally confirmed | 280 | thereof predicted | 99 |
| binders | | to bind | |
| | | thereof not predicted | 181 |
| | | to bind | |
| experimentally confirmed | 133 | thereof not predicted | 77 |
| immunogenic peptides | | | |
| experimentally confirmed | 34 | therof not predicted | 8 |
| presented peptides | | | |

TABLE 3
HLA supertype frequencies per ethnicities (tools.iedb.org/population/, Version 3.0, Accessed 2023 Feb. 24)

| | Ethnicity | | | | | | |
|---|---|---|---|---|---|---|---|
| Supertype | Caucasoid | Oriental | Mixed | Amerindian | African | Asian | WORLD |
| HLA-A1 | 43.2 | 14.7 | 43.2 | 6.0 | 35.0 | 28.6 | 35.0 |
| HLA-A2 | 50.7 | 46.2 | 47.1 | 77.4 | 42.2 | 38.6 | 46.9 |
| HLA-A3/A11 | 47.4 | 51.8 | 44.9 | 31.7 | 45.9 | 47.8 | 48.6 |
| HLA-A24 | 22.5 | 40.3 | 20.9 | 43.4 | 25.0 | 29.7 | 29.5 |
| HLA-B7 | 51.7 | 38.5 | 51.4 | 48.8 | 57.5 | 48.6 | 49.6 |
| HLA-B15 | 13.8 | 44.5 | 11.7 | 15.5 | 5.2 | 28.5 | 21.2 |
| Combinations: | | | | | | | |
| All 6 Supertypes | 99.9 | 99.6 | 99.5 | 100.0 | 98.9 | 98.9 | 99.7 |
| Combinations of 5: | | | | | | | |
| All without A1 | 96.3 | 98.9 | 94.8 | 99.9 | 94.9 | 96.3 | 97.1 |
| All without A2 | 95.0 | 95.7 | 94.0 | 87.9 | 93.5 | 94.8 | 95.3 |
| All without A3/A11 | 95.6 | 94.8 | 94.4 | 98.4 | 92.7 | 93.1 | 95.0 |
| All without A24 | 98.7 | 96.6 | 98.0 | 97.0 | 96.4 | 96.1 | 97.7 |
| All without B7 | 99.7 | 99.2 | 98.9 | 100.0 | 97.3 | 97.4 | 99.3 |
| All without B15 | 99.8 | 99.1 | 99.4 | 100.0 | 98.8 | 98.2 | 99.6 |
| Combinations of 4: | | | | | | | |
| All without A1, A2 | 85.7 | 93.9 | 83.8 | 86.5 | 85.8 | 90.1 | 88.9 |
| All without A1, | | | | | | | |
| A3/A11 | 86.8 | 92.8 | 84.6 | 97.9 | 84.7 | 87.8 | 88.5 |
| All without A1, A24 | 92.9 | 94.9 | 91.1 | 96.3 | 90.4 | 91.9 | 92.9 |
| All without A1, B7 | 91.8 | 97.8 | 88.6 | 99.8 | 87.6 | 91.5 | 93.6 |
| All without A1, B15 | 96.3 | 98.9 | 94.8 | 99.9 | 94.9 | 96.3 | 97.1 |
| All without A2, | 84.4 | 86.5 | 83.2 | 79.0 | 82.3 | 85.2 | 85.1 |
| A3/A11 | | | | | | | |
| All without A2, A24 | 91.1 | 89.3 | 90.0 | 74.5 | 88.5 | 89.8 | 90.2 |
| All without A2, B7 | 88.9 | 91.6 | 86.9 | 74.6 | 84.3 | 88.2 | 89.6 |
| All without A2, B15 | 95.0 | 95.7 | 94.0 | 87.9 | 93.5 | 94.8 | 95.3 |
| All without A3/A11, | 91.9 | 87.9 | 90.6 | 92.0 | 87.5 | 87.5 | 89.8 |
| A24 | | | | | | | |
| All without A3/A11, | 90.3 | 89.8 | 87.9 | 96.7 | 82.3 | 84.4 | 88.9 |
| B7 | | | | | | | |
| All without A3/A11, | 95.6 | 94.8 | 94.4 | 98.4 | 92.7 | 93.1 | 95.0 |
| B15 | | | | | | | |
| All without A24, B7 | 97.2 | 93.2 | 95.6 | 93.8 | 91.4 | 91.2 | 95.0 |
| All without A24, B15 | 98.7 | 96.6 | 98.0 | 97.0 | 96.4 | 96.1 | 97.7 |
| All without B7, B15 | 99.7 | 99.2 | 98.9 | 100.0 | 97.3 | 97.4 | 99.3 |

### REFERENCES

Altman et al. (1996), Science 274:94
Bassani-Sternberg et al. (2016), Nat Commun, 7, 13404. doi:10.1038/ncomms13404
Becker & Riemer (2022), Frontiers Immunol, doi: 10.3389/fimmu.2022.883989
Blatnik (2018), Proteomics 18:1700390, DOI: 10.1002/pmic.201700390
Bonsack et al. (2019), Cancer Immunol Res; 7(5):719
Bourgault Villada et al., Clin Exp Immunol 2010; 159(1):45
Chardin & McCormick (1999), Cell 97, 153-155.
Chong et al. (2018), Mol Cell Proteomics, 17(3), 533-548. doi:10.1074/mcp.TIR117.000383
Currier et al. (2002), J Immunol Methods 260, 157-172.
Habib et al. (2022), Cells 6; 11(3):421. doi: 10.3390/cells11030421
Hara et al., Int J Oncol 2005; 27(5):1371
Jang et al., Cancer 2012; 118(8):2173
Kruse et al. (2019), Oncolmmunology, 8(1):e1524694
Kast et al., J Immunol 1994; 152(8):3904
Krishna et al. (2018) Cancer Res. 2018; 78(21):6159
Lefranc et al. (2003), Dev. Comp. Immunol., 27:55
MacLean et al. (2010), Bioinformatics, 26(7), 966-968. doi:10.1093/bioinformatics/btq054
Mizuuchi et al., Exp Mol Pathol 2012; 92(1):185
Mollenhauer et al. (1990), Biochim Biophys Acta 1031, 225-246.
Ressing et al., J Immunol 1995; 154(11):5934
Riemer et al., J Biol Chem 2010; 285(38):29608
Salek et al. (2022), Analyt Bioanalyt Chem, 414(8), 2545-2552. doi:10.1007/s00216-022-03931-w
Sidney et al. (2008), BMC Immunology 9 Art. No. 1, doi.org/10.1186/1471-2172-9-1
Stanke et al. (2010), J Immunol Methods 360, 56-65.
Steinbach & Riemer (2018), Int J Cancer 142:224
Toprak et al. (2014), Mol Cell Proteomics, 13(8), 2056-2071. doi:10.1074/mcp.O113.036475
Tsang et al. (2017), Vaccine 35(19):2605
Ugel et al., Cancer Res. 2009; 69(24):9376
WO 2021/229015 A1
WO 2021/229014 A1
WO 2021/229020 A1

1. A method for inducing an immune response to a human papillomavirus 16 (HPV16)-related virus in a subject, the method comprising contacting the subject with a vaccine, wherein said vaccine provides at least five discrete immunization peptides consisting of amino acid sequences selected from at least five of the following groups:
(i) SEQ ID NOs:1 to 10;
(ii) SEQ ID NOs:11 to 52;
(iii) SEQ ID NOs:53 to 79;
(iv) SEQ ID NOs:71 and 80 to 96;
(v) SEQ ID NOs:25, 81, and 97-106; and
(vi) SEQ ID NOs: 9, 12, 23, 63, 81, 85, 101, and 107 to 131;
wherein said vaccine is a human leukocyte antigen (HLA) universal vaccine for use in any human subject expressing at least one HLA of a HLA supertype selected from the list consisting of HLA-A01, HLA-A02, HLA-A03/A11, HLA-A24, HLA-B07, and HLA-B15,
wherein each of said at least five immunization peptides, when presented as an HLA-complex, activates human anti-immunization peptide T cells,
wherein said vaccine comprises a mixture of discrete peptides each providing exactly one of said immunization peptides and/or at least one polynucleotide providing said immunization peptides in a discrete manner; and
wherein the vaccine does not comprise a mixture of six discrete and non-identical peptides comprising each exactly one amino acid sequence of SEQ ID NOs:1, 13, 54, 81, 97, and 109.

2. The method of claim 1, wherein at least one of said at least five immunogenic peptides consists of an amino acid sequence selected from group (ii) or (iii).

3. The method of claim 1, wherein said vaccine provides at least six discrete immunization peptides consisting of amino acid sequences selected from all six of said groups.

4. The method of claim 1, wherein providing an immunization peptide is causing an immunization peptide to become presented as an HLA complex on the surface of an antigen presenting cell of the human subject after contacting the human subject with said vaccine.

5. The method of claim 1, wherein said immunization peptide is linked in said vaccine to at least one of a mediator of HLA presentation, a linker, a solubility mediator, a cell-penetrating peptide, a detectable tag, a mediator of secretion, and/or an immune stimulant, or is an amphiphilic conjugate thereof.

6. The method of claim 1, wherein said vaccine comprises a mixture of discrete peptides each comprising exactly one of said amino acid sequences.

7. The method of claim 1, wherein any HPV16-derived amino acid sequence comprised in said vaccine consists of at most 12 contiguous HPV16-derived amino acids.

8. The method of claim 1, wherein said T cell is a CD8+ T cell, more preferably a cytotoxic CD8+ T cell.

9. The method of claim 1, wherein,
(i) in case the immunization peptide is selected from SEQ ID NOs: 1 to 10, the HLA supertype is HLA-A01;
(ii) in case the immunization peptide is selected from SEQ ID NOs:11 to 52, the HLA supertype is HLA-A02;
(iii) in case the immunization peptide is selected from SEQ ID NO:53 to 79, the HLA supertype is HLA-A03/A11;
(iv) in case the immunization peptide is selected from SEQ ID NOs: 71 and 80 to 96, the HLA supertype is HLA-A24;
(v) in case the immunization peptide is selected from SEQ ID NOs:25, 81, and 97-106, the HLA supertype is HLA-B07; and
(vi) in case the immunization peptide is selected from SEQ ID NO: 9, 12, 23, 63, 81, 85, 101, and 107 to 131, the HLA supertype is HLA-B15.

10. The method of claim 1, wherein a HPV16-derived peptide consisting of the same amino acid sequence as at least one of said immunization peptides is presented by HPV16-related virus-positive infected or cancer cells and wherein said at least five immunization peptides are selected from at least five of the following groups:
(i) SEQ ID NOs: 1 to 2;
(ii) SEQ ID NOs:11 to 23;
(iii) SEQ ID NOs:53 to 57;
(iv) SEQ ID NOs: 80 to 81;
(v) SEQ ID NOs: 81, 97, and 98; and
(vi SEQ ID NOs: 81, and 107-114.

11. The method of claim 1, wherein said HLA universal vaccine provides immunization peptides such that at least 90% of subjects of the world's population express at least one HLA capable of presenting at least one of said immunization peptides.

12. The method of claim 1, comprising at least one polynucleotide providing said immunization peptides in a discrete manner, or being a nanoemulsion comprising said mixture of discrete peptides.

13. A kit comprising the vaccine as specified in claim 1 comprised in a housing.

14. (canceled)

15. A method for stimulating T cells recognizing HPV16-related virus-infected host cells (anti-HPV16 T cells), said method comprising
(i) contacting an agent expressing MHC class I molecules with a vaccine as specified in claim 1, thereby causing presentation of at least one of said immunization peptides on said MHC class I molecules,
(ii) contacting the agent expressing MHC class I molecules of step (i) with T cells, and, thereby, stimulating anti-HPV16 T cells.


SEQUENCE LISTING (148 sequences)

SEQ ID NO: 1 - protein, 9 aa, Human papillomavirus 16
ISEYRHYCY

SEQ ID NO: 2 - protein, 10 aa, Human papillomavirus 16
HGDTPTLHEY

SEQ ID NO: 3 - protein, 11 aa, Human papillomavirus 16
TIHDIILQCVY

SEQ ID NO: 4 - protein, 11 aa, Human papillomavirus 16
YGVCDKCLKFY

SEQ ID NO: 5 - protein, 10 aa, Human papillomavirus 16
EYRYYCYSLY

SEQ ID NO: 6 - protein, 10 aa, Human papillomavirus 16
DLQPETTDLY

SEQ ID NO: 7 - protein, 9 aa, Human papillomavirus 16
TTDLYCYEQ

SEQ ID NO: 8 - protein, 11 aa, Human papillomavirus 16
SKISEYRHYCY

SEQ ID NO: 9 - protein, 11 aa, Human papillomavirus 16
SEYRHYCYSLY

SEQ ID NO: 10 - protein, 8 aa, Human papillomavirus 16
ETTDLYCY

SEQ ID NO: 11 - protein, 10 aa, Human papillomavirus 16
TIHDIILECV

SEQ ID NO: 12 - protein, 9 aa, Human papillomavirus 16
TLHEYMLDL

SEQ ID NO: 13 - protein, 9 aa, Human papillomavirus 16
YMLDLQPET

SEQ ID NO: 14 - protein, 10 aa, Human papillomavirus 16
YMLDLQPETT

SEQ ID NO: 15 - protein, 11 aa, Human papillomavirus 16
YMLDLQPETTD

SEQ ID NO: 16 - protein, 8 aa, Human papillomavirus 16
MLDLQPET

SEQ ID NO: 17 - protein, 9 aa, Human papillomavirus 16
RLCVQSTHV

SEQ ID NO: 18 - protein, 10 aa, Human papillomavirus 16
RTLEDLLMGT

SEQ ID NO: 19 - protein, 11 aa, Human papillomavirus 16
RTLEDLLMGTL

SEQ ID NO: 20 - protein, 9 aa, Human papillomavirus 16
TLEDLLMGT

SEQ ID NO: 21 - protein, 11 aa, Human papillomavirus 16
EDLLMGTLGIV

SEQ ID NO: 22 - protein, 10 aa, Human papillomavirus 16
DLLMGTLGIV

SEQ ID NO: 23 - protein, 9 aa, Human papillomavirus 16
LLMGTLGIV

SEQ ID NO: 24 - protein, 11 aa, Human papillomavirus 16
DLLMGTLGIVC

SEQ ID NO: 25 - protein, 11 aa, Human papillomavirus 16
LMGTLGIVCPI

SEQ ID NO: 26 - protein, 9 aa, Human papillomavirus 16
KLPQLCTEL

SEQ ID NO: 27 - protein, 9 aa, Human papillomavirus 16
KLPDLCTEL

SEQ ID NO: 28 - protein, 9 aa, Human papillomavirus 16
MLDLQPETT

SEQ ID NO: 29 - protein, 9 aa, Human papillomavirus 16
GTLGIVCPI

SEQ ID NO: 30 - protein, 8 aa, Human papillomavirus 16
TLGIVCPI

SEQ ID NO: 31 - protein, 9 aa, Human papillomavirus 16
TLGIVCPIC

SEQ ID NO: 32 - protein, 8 aa, Human papillomavirus 16
YMLDLQPE

SEQ ID NO: 33 - protein, 10 aa, Human papillomavirus 16
MGTLGIVCPI

SEQ ID NO: 34 - protein, 9 aa, Human papillomavirus 16
ELQTTIHEI

SEQ ID NO: 35 - protein, 11 aa, Human papillomavirus 16
TTIHEIILECV

SEQ ID NO: 36 - protein, 11 aa, Human papillomavirus 16
TTIHEIRLECV

SEQ ID NO: 37 - protein, 10 aa, Human papillomavirus 16
TIHEIILECV

SEQ ID NO: 38 - protein, 10 aa, Human papillomavirus 16
TIHEIRLECV

SEQ ID NO: 39 - protein, 10 aa, Human papillomavirus 16
SEYRYYCYSL

SEQ ID NO: 40 - protein, 10 aa, Human papillomavirus 16
SEYRYYCYSV

SEQ ID NO: 41 - protein, 8 aa, Human papillomavirus 16
YRYYCYSV

SEQ ID NO: 42 - protein, 10 aa, Human papillomavirus 16
RYYCYSLYGT

SEQ ID NO: 43 - protein, 10 aa, Human papillomavirus 16
LLMGTLGIVC

SEQ ID NO: 44 - protein, 11 aa, Human papillomavirus 16
LLMGTLGIVCP

SEQ ID NO: 45 - protein, 9 aa, Human papillomavirus 16
FQDPQERPI

SEQ ID NO: 46 - protein, 11 aa, Human papillomavirus 16
FQDPQERPIKL

SEQ ID NO: 47 - protein, 11 aa, Human papillomavirus 16
KLPQLCTELQT

SEQ ID NO: 48 - protein, 9 aa, Human papillomavirus 16
ELQTTIHDI

SEQ ID NO: 49 - protein, 11 aa, Human papillomavirus 16
TTIHDIILECV

SEQ ID NO: 50 - protein, 8 aa, Human papillomavirus 16
YRYYCYSL

SEQ ID NO: 51 - protein, 11 aa, Human papillomavirus 16
TLHEYMLDLQP

SEQ ID NO: 52 - protein, 11 aa, Human papillomavirus 16
IRTLEDLLMGT

SEQ ID NO: 53 - protein, 10 aa, Human papillomavirus 16
CVYCKQQLLR

SEQ ID NO: 54 - protein, 9 aa, Human papillomavirus 16
TTLEQQYNK

SEQ ID NO: 55 - protein, 9 aa, Human papillomavirus 16
IVCPICSQK

SEQ ID NO: 56 - protein, 9 aa, Human papillomavirus 16
IILECVYCK

SEQ ID NO: 57 - protein, 9 aa, Human papillomavirus 16
HLDKKQRFH

SEQ ID NO: 58 - protein, 11 aa, Human papillomavirus 16
HEIILECVYCK

SEQ ID NO: 59 - protein, 11 aa, Human papillomavirus 16
FAFRDLCIVYR

SEQ ID NO: 60 - protein, 10 aa, Human papillomavirus 16
AFRDLCIVYR

SEQ ID NO: 61 - protein, 9 aa, Human papillomavirus 16
IVYRDGNPY

SEQ ID NO: 62 - protein, 11 aa, Human papillomavirus 16
NPYAVCDKCLK

SEQ ID NO: 63 - protein, 10 aa, Human papillomavirus 16
AVCDKCLKFY

SEQ ID NO: 64 - protein, 11 aa, Human papillomavirus 16
AVCDKCLKFYS

SEQ ID NO: 65 - protein, 11 aa, Human papillomavirus 16
VCDKCLKFYSK

SEQ ID NO: 66 - protein, 10 aa, Human papillomavirus 16
GIVCPICSQK

SEQ ID NO: 67 - protein, 11 aa, Human papillomavirus 16
MFQDPQERPRK

SEQ ID NO: 68 - protein, 11 aa, Human papillomavirus 16
YAVCDKCLKFY

SEQ ID NO: 69 - protein, 8 aa, Human papillomavirus 16
AVCDKCLK

SEQ ID NO: 70 - protein, 9 aa, Human papillomavirus 16
KCLKFYSKI

SEQ ID NO: 71 - protein, 9 aa, Human papillomavirus 16
KFYSKISEY

SEQ ID NO: 72 - protein, 10 aa, Human papillomavirus 16
KFYSKISEYR

SEQ ID NO: 73 - protein, 11 aa, Human papillomavirus 16
RHYCYSVYGTT

SEQ ID NO: 74 - protein, 11 aa, Human papillomavirus 16
DLLIRCINCQK

SEQ ID NO: 75 - protein, 10 aa, Human papillomavirus 16
LLIRCINCQK

SEQ ID NO: 76 - protein, 9 aa, Human papillomavirus 16
LIRCINCQK

SEQ ID NO: 77 - protein, 11 aa, Human papillomavirus 16
RCINCQKPLCP

SEQ ID NO: 78 - protein, 10 aa, Human papillomavirus 16
KQRFHNIRGR

SEQ ID NO: 79 - protein, 11 aa, Human papillomavirus 16
LGIVCPICSQK

SEQ ID NO: 80 - protein, 8 aa, Human papillomavirus 16
VYCKQQLL

SEQ ID NO: 81 - protein, 9 aa, Human papillomavirus 16
RAHYNIVTF

SEQ ID NO: 82 - protein, 9 aa, Human papillomavirus 16
EYRHYCYSL

SEQ ID NO: 83 - protein, 10 aa, Human papillomavirus 16
TFCCKCDSTL

SEQ ID NO: 84 - protein, 11 aa, Human papillomavirus 16
PYAVCDKCLKF

SEQ ID NO: 85 - protein, 11 aa, Human papillomavirus 16
SEYRHYCYSVY

SEQ ID NO: 86 - protein, 10 aa, Human papillomavirus 16
VYGTTLEQQY

SEQ ID NO: 87 - protein, 11 aa, Human papillomavirus 16
PDRAHYNIVTF

SEQ ID NO: 88 - protein, 10 aa, Human papillomavirus 16
DRAHYNIVTF

SEQ ID NO: 89 - protein, 9 aa, Human papillomavirus 16
HYNIVTFCC

SEQ ID NO: 90 - protein, 8 aa, Human papillomavirus 16
TFCCKCDF

SEQ ID NO: 91 - protein, 10 aa, Human papillomavirus 16
VYRDGNPYGV

SEQ ID NO: 92 - protein, 11 aa, Human papillomavirus 16
HYCYSLYGTTL

SEQ ID NO: 93 - protein, 8 aa, Human papillomavirus 16
YSLYGTTL

SEQ ID NO: 94 - protein, 11 aa, Human papillomavirus 16
HLDKKQRFHNI

SEQ ID NO: 95 - protein, 8 aa, Human papillomavirus 16
DKKQRFHN

SEQ ID NO: 96 - protein, 8 aa, Human papillomavirus 16
AHYNIVTF

SEQ ID NO: 97 - protein, 8 aa, Human papillomavirus 16
RPRKLPQL

SEQ ID NO: 98 - protein, 9 aa, Human papillomavirus 16
TPTLHEYML

SEQ ID NO: 99 - protein, 11 aa, Human papillomavirus 16
RPRKLPQLCTE

SEQ ID NO: 100 - protein, 9 aa, Human papillomavirus 16
LEQQYNKPL

SEQ ID NO: 101 - protein, 11 aa, Human papillomavirus 16
NIRGRWTGRCM

SEQ ID NO: 102 - protein, 10 aa, Human papillomavirus 16
EPDRAHYNIV

SEQ ID NO: 103 - protein, 9 aa, Human papillomavirus 16
RPRKLPQLC

SEQ ID NO: 104 - protein, 10 aa, Human papillomavirus 16
RPRKLPQLCT

SEQ ID NO: 105 - protein, 8 aa, Human papillomavirus 16
KPLCDLLI

SEQ ID NO: 106 - protein, 10 aa, Human papillomavirus 16
RCMSCCRSSR

SEQ ID NO: 107 - protein, 10 aa, Human papillomavirus 16
KQQLLRREVY

SEQ ID NO: 108 - protein, 9 aa, Human papillomavirus 16
AFRDLCIVY

SEQ ID NO: 109 - protein, 9 aa, Human papillomavirus 16
LQPETTDLY

SEQ ID NO: 110 - protein, 10 aa, Human papillomavirus 16
GQAEPDRAHY

SEQ ID NO: 111 - protein, 9 aa, Human papillomavirus 16
QQLLRREVY

SEQ ID NO: 112 - protein, 11 aa, Human papillomavirus 16
SVYGTTLEQQY

SEQ ID NO: 113 - protein, 10 aa, Human papillomavirus 16
LQPETTDLYC

SEQ ID NO: 114 - protein, 11 aa, Human papillomavirus 16
LQPETTDLYCY

SEQ ID NO: 115 - protein, 10 aa, Human papillomavirus 16
FAFRDLCIVY

SEQ ID NO: 116 - protein, 11 aa, Human papillomavirus 16
LCIVYRDGNPY

SEQ ID NO: 117 - protein, 9 aa, Human papillomavirus 16
YRHYCYSVY

SEQ ID NO: 118 - protein, 11 aa, Human papillomavirus 16
LLRREVYDFAF

SEQ ID NO: 119 - protein, 10 aa, Human papillomavirus 16
LRREVYDFAF

SEQ ID NO: 120 - protein, 11 aa, Human papillomavirus 16
IVYRDGNPYAV

SEQ ID NO: 121 - protein, 9 aa, Human papillomavirus 16
AVCDKCLKF

SEQ ID NO: 122 - protein, 11 aa, Human papillomavirus 16
CLKFYSKISEY

SEQ ID NO: 123 - protein, 8 aa, Human papillomavirus 16
FYSKISEY

SEQ ID NO: 124 - protein, 10 aa, Human papillomavirus 16
YSKISEYRHY

SEQ ID NO: 125 - protein, 9 aa, Human papillomavirus 16
SKISEYRHY

SEQ ID NO: 126 - protein, 8 aa, Human papillomavirus 16
SEYRHYCY

SEQ ID NO: 127 - protein, 10 aa, Human papillomavirus 16
QQYNKPLCDL

SEQ ID NO: 128 - protein, 9 aa, Human papillomavirus 16
CQKPLCPEE

SEQ ID NO: 129 - protein, 11 aa, Human papillomavirus 16
KQRHLDKKQRF

SEQ ID NO: 130 - protein, 11 aa, Human papillomavirus 16
KQRFHNIRGRW

SEQ ID NO: 131 - protein, 9 aa, Human papillomavirus 16
GQAEPDRAH

SEQ ID NO: 132 - protein, 10 aa, Human papillomavirus 16
TTDLYCYEQL

SEQ ID NO: 133 - protein, 8 aa, Human papillomavirus 16
SEYRHYCY

SEQ ID NO: 134 - protein, 10 aa, Human papillomavirus 16
IHEIILECVY

SEQ ID NO: 135 - protein, 10 aa, Human papillomavirus 16
GVCDKCLKFY

SEQ ID NO: 136 - protein, 10 aa, Human papillomavirus 16
GTTLEQQYNK

SEQ ID NO: 137 - protein, 8 aa, Human papillomavirus 16
RHYCYSLY

SEQ ID NO: 138 - protein, 8 aa, Human papillomavirus 16
HYNIVTFC

SEQ ID NO: 139 - protein, 10 aa, Human papillomavirus 16
TFCCKCDFTL

SEQ ID NO: 140 - protein, 8 aa, Human papillomavirus 16
RPGKLPQL

SEQ ID NO: 141 - protein, 10 aa, Human papillomavirus 16
RPRKLPDLCT

SEQ ID NO: 142 - protein, 9 aa, Human papillomavirus 16
CPDEKQRHL

SEQ ID NO: 143 - protein, 10 aa, Human papillomavirus 16
LPQLCTELQT

SEQ ID NO: 144 - protein, 8 aa, Human papillomavirus 16
AHYNIVTF

SEQ ID NO: 145 - protein, 8 aa, Human papillomavirus 16
KISEYRHY

SEQ ID NO: 146 - protein, 9 aa, Human papillomavirus 16
RGRWTGRCM

SEQ ID NO: 147 - protein, 11 aa, Human papillomavirus 16
AGQAEPDRAHY

SEQ ID NO: 148 - protein, 8 aa, Human papillomavirus 16
FYSKISEY